(19) United States
(12) Patent Application Publication
(10) Pub. No.: US 2023/0355815 A1
(43) Pub. Date: Nov. 9, 2023

(54) ACTIVE DELIVERY OF RADIOTRACERS ACROSS THE BLOOD BRAIN BARRIER

(71) Applicant: The Board of Trustees of the Leland Stanford Junior University, Stanfod, CA (US)

(72) Inventors: Michelle L. JAMES, Menlo Park, CA (US); Katrin ANDREASSON, Palo Alto, CA (US); Israt ALAM, Stanford, CA (US)

(21) Appl. No.: 18/339,653

(22) Filed: Jun. 22, 2023

Related U.S. Application Data
(63) Continuation of application No. PCT/US2022/012569, filed on Jan. 14, 2022.
(60) Provisional application No. 63/137,543, filed on Jan. 14, 2021.
(60) Provisional application No. 63/298,393, filed on Jan. 11, 2022.

(51) Int. Cl. A61K 51/10; A61K 49/16; A61P 37/00

(52) U.S. Cl. CPC A61K 51/1027; A61K 49/16; A61P 37/00

(57) ABSTRACT

Non-invasive methods permitting accurate detection of innate immune dysfunction are critical to enhance the understanding, diagnosis, and treatment of inflammatory disorders. Identifying specific biomarkers of innate immune cells and their functional phenotypes, paired with subsequent PET tracer development, is thus an area of great interest with important implications for a broad range of diseases. However, existing targets lack cell specificity and/or fail to provide functionally relevant information regarding immune cell status. To overcome these limitations, a PET tracer was developed targeting TREM1 a highly specific biomarker of pro-inflammatory myeloid-driven immune responses.

### CROSS-REFERENCE TO RELATED APPLICATIONS

This application is a continuation of International Application No. PCT/US2022/012569, filed Jan. 14, 2022, which claims benefit of U.S. Provisional Application No. 63/137,543, filed on Jan. 14, 2021, and to U.S. Provisional Application 63/298,393, filed Jan. 11, 2022, the entireties of which are hereby incorporated by reference.

### STATEMENT REGARDING FEDERALLY SPONSORED RESEARCH OR DEVELOPMENT

This invention was made with government support under grant numbers 5R01NS04572711 awarded by National Institutes of Neurological Disorders and Stroke. The government has certain rights in this invention.

### FIELD

Provided herein are efficient methods for delivering one or more imaging or diagnostic substance(s) to the brain.

### BACKGROUND

The concept of the blood brain barrier (“BBB”) was discovered by Paul Ehrlich in 1885. Erhlich was a Nobel prize-winning German physician and scientist studying staining. Ehrlich injected aniline into animals and noticed that the dyes stained all of the organs of some kinds of animals except for their brains. In 1913, one of Ehrlich's students, Edwin Goldman, injected the dye into the cerebrospinal fluids of animals' brains directly. Goldman found that although the brains became dyed, the rest of the body did not. The experiments demonstrated the existence of some sort of compartmentalization between the brain and the rest of the body. The BBB was observed and shown to exist with the introduction of the scanning electron microscope in the 1960s.

The BBB is a highly selective semipermeable membrane border of endothelial cells that prevents solutes in circulating blood from non-selectively crossing into the fluid of the central nervous system. At the interface between the blood and the brain, endothelial cells are joined with tight junctions. The tight junctions are composed of smaller subunits of transmembrane proteins, such as occludin, claudins, and junctional adhesion molecules. The selectivity of the BBB is the result of the tight junctions formed between endothelial cells of brain capillaries that restrict the passage of solutes.

The transmembrane proteins are anchored into the endothelial cells by another protein complex that includes tight junction protein 1 and associated proteins. Astrocyte cell projections, called astrocytic feet, surround the endothelial cells and provide biochemical support to the endothelial cells. Pericytes are embedded in the capillary basement membrane.

The BBB allows the passage of some molecules by passive diffusion as well as the selective active transport of various nutrients, ions, organic anions, and macromolecules such as glucose, water, and amino acids that are crucial to neural function. The BBB restricts the passage of pathogens, the diffusion of solutes in blood, and large or hydrophilic molecules, such as antibodies, into cerebrospinal fluid. The BBB restricts the passage of substances more selectively than endothelial cells of capillaries elsewhere in the body.

Delivery of diagnostic molecules to the brain is especially challenging because it must take into account the special anatomy of the brain as well as the restrictions imposed by the special junctions of the BBB. Invasive methods have been developed to directly deliver molecules to and from the brain such as, for example, brain microdialysis, intracerebral implantation, and intraventricular delivery. But, invasive methods can be problematic for patients as the methods may cause damage in surrounding tissues. Non-invasive methods have also been developed such as, for example, prodrug technologies, efflux pump inhibitors, receptor-mediated transport, osmotic agents, and BBB modulators. The success of current non-invasive methods has also been limited.

What is needed is an efficient method or composition for delivering one or more substance(s) to the brain.

### SUMMARY

A first aspect provides a method of delivering one or more imaging or diagnostic substance(s) to the brain in a subject in need thereof, comprising administering an antigen binding molecule to the subject, wherein the antigen binding molecule binds an antigen in peripheral immune cells. In some embodiments, the peripheral immune cells are myeloid cells, NK cells, macrophages, monocytes, granulocytes, dendritic cells, and/or inflammatory cells that pass through the brain. In some embodiments, the granulocytes are one or more of neutrophil(s), eosinophil(s), and basophil(s).

In some embodiments, the subject is in need of diagnosis. In some embodiments, the delivery of the one or more substances to the brain identifies the location of inflammation associated with a disease or condition. In some embodiments, the disease or condition comprises one or more of multiple sclerosis, Alzheimer's disease, Huntington's disease, Parkinson's disease, epilepsy, brain tumor, stroke, amyotrophic lateral sclerosis, spinal cord and/or brain trauma, a disease or condition which would benefit from enzyme replacement therapy (“ERT”), a neurological disease, chronic inflammatory conditions, acute inflammatory conditions, or bacterial infection. In some embodiments, the disease or condition comprises autoimmune diseases and infection. In some embodiments, the disease or condition comprises frontotemporal dementia (“FTD”), viral infections, and parasitic infections. In some embodiments, the viral infections comprise one or more of HIV or West Nile Virus. In some embodiments, parasitic infections comprise one or more of schistosomiasis and/or trypanosomiasis.

In some embodiments, the one or more substance(s) are different from the antigen binding molecule and the one or more substance(s) is linked to the antigen binding molecule. In some embodiments, the antigen binding molecule and/or one or more substance(s) is one or more of antibody(s), antibody fragment(s), biologic(s), peptides, small molecule(s), engineered protein scaffold(s), a nucleic acid, or a CRISPR-Cas9 molecule. In some embodiments, the nucleic acid is RNA and/or DNA. In some embodiments, the nucleic acid is an antisense oligonucleotide (See, for example, Brenner et al. Gene Specific Therapies—the next therapeutic milestone in neurology. Neurological Research and Practice 2: 25 (2020); and Quemener et al. The powerful world of antisense oligonucleotides: From bench to bedside. WIREs RNA 11:e 1594 (2020), each of which is incorporated in their entirety herein).

In some embodiments, the one or more substance(s) comprises one or more diagnostic or substance(s). In some embodiments, the one or more diagnostic substance(s) comprises one or more PET and/or MRI probe(s), radiolabel(s), or isotope(s).

In some embodiments, the antigen is TREM1, TREM2, GPR84, (inducible) toll-like receptor, or a nucleotide-binding oligomerization domain-like receptor. In some embodiments, the subject is human. In some embodiments, the administration is through IV, intramuscular, subcutaneous, intraperitoneal, intravitreal, or intrathecal.

A second aspect of the invention provides a composition for delivering one or more substance(s) to the brain in a subject in need thereof, the composition comprising an antigen binding molecule, wherein the antigen binding molecule binds an antigen expressed on peripheral immune cells. In some embodiments, the peripheral immune cells are myeloid cells, NK cells, macrophages, monocytes, granulocytes, dendritic cells, and/or inflammatory cells that pass through the brain. In some embodiments, the granulocytes are one or more of neutrophil(s), eosinophil(s), and basophil(s).

In some embodiments, the subject is in need of diagnosis. In some embodiments, the diagnosis comprises diagnosis of a disease or condition. In some embodiments, the delivery of the one or more substances to the brain identifies the location of inflammation associated with a disease or condition. In some embodiments, the disease or condition comprises one or more of multiple sclerosis, Alzheimer's disease, Huntington's disease, Parkinson's disease, epilepsy, brain tumor, stroke, amyotrophic lateral sclerosis, spinal cord and/or brain trauma, a disease or condition which would benefit from enzyme replacement therapy (“ERT”), a neurological disease, chronic inflammatory conditions, acute inflammatory conditions, or bacterial infection. In some embodiments, the disease or condition comprises autoimmune diseases and infection. In some embodiments, the disease or condition comprises autoimmune diseases and infection. In some embodiments, the disease or condition comprises frontotemporal dementia (“FTD”), viral infections, and parasitic infections. In some embodiments, the viral infections comprise one or more of HIV or West Nile Virus. In some embodiments, parasitic infections comprise one or more of schistosomiasis and/or trypanosomiasis.

In some embodiments, the one or more substance(s) are different from the antigen binding molecule and the one or more substance(s) is linked to the antigen binding molecule. In some embodiments, the antigen binding molecule and/or one or more substance(s) is one or more of antibody(s), antibody fragment(s), biologic(s), peptide(s), small molecule(s), engineered protein scaffold(s), a nucleic acid, or a CRISPR-Cas9 molecule. In some embodiments, the nucleic acid is RNA and/or DNA. In some embodiments, the nucleic acid is an antisense oligonucleotide.

In some embodiments, the one or more substance(s) comprises or consists of one or more diagnostic substance(s). In some embodiments, the one or more diagnostic substance(s) comprises or consists of PET and/or MRI probe(s), radiolabel(s) or isotope(s).

In some embodiments, the antigen is TREM1, TREM2, GPR84, (inducible) toll-like receptor, or a nucleotide-binding oligomerization domain-like receptor. In some embodiments, the subject is human. In some embodiments, the administration is through IV, intramuscular, subcutaneous, intraperitoneal, intravitreal, or intrathecal.

A third aspect provides a nucleic acid encoding any of the compositions or the antigen binding molecules set forth herein. In some embodiments, the nucleic acid comprises a vector. Some embodiments provide a host transformed with the vector. Some embodiments provide a method for the production of one or more composition(s) or the antigen binding molecules comprising the steps of expressing any of the nucleic acid provided herein in a prokaryotic or eukaryotic host cell and recovering the one or more composition(s) or the antigen binding molecules from the cell or the cell culture supernatant.

### DESCRIPTION OF DRAWINGS

FIG. 1A shows 3D sagittal maximum intensity projection PET images of representative sham and MCAo stroke mice 1.5-2 days post-surgery injected with either [⁶⁴Cu]TREM1-mAb or [⁶⁴Cu]Isotype-control-mAb. FIG. 1B shows quantitation of PET signal in spleen and FIG. 1C shows quantitation of PET signal in intestines, both showing a higher uptake of [⁶⁴Cu]TREM1-mAb in MCAo mice (n=12) compared to sham mice (n=10). FIG. 1D shows in vivo brain PET quantitation reveals significantly higher [⁶⁴Cu]TREM1-mAb signal within the infarct of MCAo mice, compared to uptake in a corresponding contralateral brain region and also uptake in an equivalent brain region from Sham mice (n=9 per group). FIG. 1E shows quantification of ex vivo brain autoradiography (AR) of mice imaged with [⁶⁴Cu]TREM1-mAb or [⁶⁴Cu]isotype-control-mAb (n=9-10 biologically independent samples per group, mean±s.e.m.; two tailed Student's unpaired t-test, *P<0.05). FIG. 1F shows a representative autoradiography images of coronal brain sections, cresyl violet staining and overlay of autoradiography and cresyl staining from mice imaged with [⁶⁴Cu]TREM1-mAb or [⁶⁴Cu]isotype-control-mAb 36 h after MCAo.

FIG. 2 shows the results from tracking of peripheral infiltrating activated myeloid cells with TREM1-PET in a mouse model of chronic/progressive multiple sclerosis (experimental autoimmune encephalomyelitis, EAE model). Representative images show that TREM1-PET can visualize markedly elevated tracer uptake in the spleen, bone marrow, spinal cord, and brain of EAE versus naïve mice and TREM1 knockout (KO) EAE mice.

FIG. 3 shows that quantitation of the TREM1-PET signal is able to detect pro-inflammatory peripheral CNS-infiltrating myeloid cells in EAE mice. TREM1-PET signal in the spinal cord and brain regions is significantly higher in EAE versus naïve and TREM1 KO EAE mice.

FIG. 4 shows that TREM1-PET is more sensitive than TSPO-PET at detecting toxic inflammation in EAE. TSPO-PET is unable to delineate activated myeloid cells in the lumbar and thoracic spinal cord as clearly as TREM1-PET.

FIG. 5 shows the study design for assessing TREM1-PET as a tool to monitor disease in RR-EAE.

FIG. 6 shows TREM1-PET provides sensitive monitoring of relapses and remissions in EAE. The left side of the figure shows [⁶⁴Cu] TREM1-mAb images in RR-EAE mice, with the top showing in-vivo PET/CT images and the bottom showing spinal cord autoradiography. The right side of the figure shows TREM1-PET quantification with the top (FIGS. 6A and 6B) showing quantification of TREM1-PET signal in the lumbar/spinal cord and the bottom (FIGS. 6C and 6D) showing TREM1-PET signal in the cervical/thoracic spinal cord.

FIGS. 7A-7D show TREM1-PET provides sensitive monitoring of relapses and remissions in EAE. FIG. 7A shows TREM1-PET quantification of whole brain; FIG. 7B shows TREM1-PET quantification of the medulla; FIG. 7C shows TREM1-PET quantification of whole pons; and FIG. 7D shows TREM1-PET quantification of the cerebellum.

FIG. 8 shows TREM1-PET imaging enables highly specific in vivo detection of innate immune activation in a mouse model of LPS-induced sepsis. [⁶⁴Cu]TREM1-mAb PET/CT image of a vehicle control mouse (left) and an LPS injected mouse (center) and [⁶⁴Cu]isotype-control PET/CT image of a LPS injected mouse (right).

FIG. 9 further demonstrates that TREM-1 is a specific tool for detecting activated myeloid cells after LPS challenge using quantification of PET images (FIG. 9A, left), ex vivo biodistribution data (FIG. 9A, right) and autoradiography (FIG. 9B) from the different groups.

FIGS. 10A and 10B shows TREM1-PET imaging can detect subtle neuroinflammation (i.e., peripheral CNS-infiltrating pro-inflammatory myeloid cells) in the brain of a mouse model of sepsis.

FIG. 11 shows elevated TREM1-PET signal in brain of APPSwe vs. age-matched (10 month old) wild-type mice. Images demonstrate increased binding of the TREM1-PET tracer in the choroid plexus, ventricles, hippocampus and the cortical regions of the APPSwe transgenic mice compared to the age/sex-matched wild type mice.

FIG. 12 shows increased [⁶⁴Cu] TREM1-mAb signal in hippocampus of 5×FAD compared to age-matched (6 month old) wild-type mice. TREM1-PET and autoradiography of 5×FAD and wild-type (WT) mice.

FIG. 13A Whole body representative 3D maximum intensity projection PET/CT images of Veh-WT, LPS-WT, and LPS-K/O mice 20 h after injection of 64Cu-TREM1-mAb in addition to LPS-WT mice 20 h after injection of 64Cu-Isotype-control-mAb (LPS-ISO-WT). FIG. 13B Quantification of PET images FIG. 13C and ex vivo gamma-counting of liver, lung, and spleen. Statistical analysis performed using one-way ANOVAs.

FIG. 14. Autoradiography of spleen sections from Veh-WT, LPS-WT, LPS-ISO, and LPS/K/O mice. H&E staining overlaid with autoradiography reveals tracer binding is restricted to the marginal zone and red pulp which contain macrophages.

FIG. 15. Proportions of myeloid and lymphoid cell subpopulations within the spleen and lungs of LPS-WT, Veh-WT wild-type C57BL/6, mice and LPS-K/O TREm1 knockout mice. Percentage of TREM1·cells as a proportion of all live single cells, as well as within each parent population (i.e., myeloid and lymphoid), in the spleen and lungs. *vs VehWT, +vs LPS-K/O. **p<0.01, ***p<0.001, ****p<0.0001. Data are representative of at least 2 independent experiments.

FIG. 16A Quantification of PET signal in brain regions of interest was performed using a segmented 3D mouse brain atlas and coronal brain PET/CT images from Veh WT, LPS-WT, LPS-ISO-WT, and LPS-K/O mice 20 h post-injection of tracer. Quantification of % ID/g in whole brain using FIG. 16B PET images and FIG. 16C gamma counting (one-way ANOVAs). FIG. 16D Quantification of PET signal in segmented brain regions of interest (2-way ANOVA). *vs Veh-WT, #vs LPS-ISO, +vs LPS-K/O (*p<0.05, **p<0.01, ***p<0.001, ****p<0.0001). Data expressed as meant SD.

FIG. 17. Representative autoradiography (autorad) images and nissl staining of coronal brain sections from Veh-WT, LPS-WT, LPS-ISO-WT, and LPS-K/O mice 20 h post-injection of 64Cu-TREM1-mAb or 64Cu-Isotype control with nissl staining for anatomical correlation.

FIG. 18. Proportions of myeloid and lymphoid cell subpopulations within the brain of LPS-WT, Veh-WT, and LPS-K/O mice. Percentage of TREM1+ cells as a proportion of all live single cells, as well as within each parent population (i.e., microglia, myeloid, and lymphoid) in the brain. *vs Veh-Wr, +vs LPS-K/O. (*p<0.05, ***p<0.001, ****p<0.001). Data are representative of at least 2 independent experiments.

FIG. 19A Log 2 fold change heat map representation of cytokines (rows) for LPStreated and saline-treated wild-type (WT) mice (columns) normalized to vehicle average. Row/column order are the results of unsupervised hierarchical clustering. Resulting three primary cytokine clusters 1-3. Correlogram of PET signal (% ID/g of 64Cu-TREM 1-mAb uptake) in brain (medulla), lung, and spleen versus plasma cytokine levels. FIG. 19B Circle size and color denote Pearson's correlation coefficient. Venn diagram of shared biological ontology annotations (ENSEMBL database) between clusters. Fraction of genes in cluster with shared annotation. Significant hits identified via over-representation analysis (onesided Fishers Exact Test) and annotations shared by greater than 40% of cytokines in cluster 1 are depicted (*p<0.05). FIG. 19C Kaplan-Meier survival curves for TREM1 K/0 or WT mice treated with 15 mg/kg LPS. Log-rank hazard ratio and Log-rank (Mantel-Cox) test *p<0.05. Appearance and activity levels of TREM1 K/0 and Wr mice post-LPS challenge based on an adapted murine sepsis scoring system 55 (unpaired t-tests per time-point). (*p<0.05, **p<0.01).

FIG. 20. Translational imaging approach for the detection of activated peripheral CNS infiltrating myeloid cells. Peripheral myeloid cells (i.e., monocytes, macrophages, neutrophils and dendritic cells) are recruited to the central nervous system (CNS) during multiple sclerosis (MS). Peripheral myeloid cells, in addition to brain resident microglia, are associated with CNS MS lesions and are fundamental to disease progression and remission. We have identified triggering receptor on myeloid cells 1 (TREM1) positron emission tomography (PET) as a novel approach to detect pathogenic peripheral CNS-infiltrating myeloid cells in the experimental autoimmune encephalomyelitis (EAE) mouse using [⁶⁴Cu]TREM1-mAb whole-body PET/CT.

FIGS. 21A-21I. TREM1+ peripheral myeloid cell expansion and CNS-infiltration is observed in EAE: from a pre-symptomatic disease state. EAE was induced in C57/bl6 and Treml knockout (KO) mice. Brain, spinal cord and spleen tissues were harvested for single cell flow cytometry at different disease states (FIG. 21A) Representative expansion of TREMl c o45hiCDII b+ myeloid cells in the spinal cord of naive, pre EAE, low EAE, high EAE, and Trem 1 KO EAE mice (FIG. 21B). Representative histogram of c o45in+c o 11 b+ microglia, CD45hiCD 11 b+ myeloid, and CD45+c o 11 b-lymphoid cell populations in the spinal cord of a low EAE mouse (FIG. 21C). Populations of peripheral myeloid, microglia, and lymphoid cells within the spleen (FIG. 21D), spinal cord (FIG. 21E), and whole brain (FIG. 21F) of naive and EAE (pre, low, high and KO) mice. Percentage of TREMl+ cells (frequency of all live single cells) in the spleen (FIG. 21G), spinal cord (FIG. 21H) and brain (FIG. 21I). Statistical analysis was performed using a 2-way ANOVA and Tukey's multiple comparison test. +vs. na'ive mice. #vs. KO EAE mice, and * denotes direct comparison of groups (+P:S 0.05, ++P:S 0.01. ++++P:S 0.0001, 1 #1 P:S 0.01, ###p:S 0.001, ####p:S 0.0001. ****P=0.0001).

FIGS. 22A-22L show in vivo TREMl PET enables early detection of disease in EAE. PET/CT was performed 20 h following [⁶⁴Cu]TREM1-mAb injection and tissues were harvested for ex vivo analysis FIG. 22A. TREMi-PET images highlighting elevated signal in the spinal cord (arrow), spleen (outline), and bone of EAE mice versus Treml knockout (KO) EAE and naive mice FIG. 22B. Quantification of [⁶⁴Cu]TREMl-mAb PET images in lumbar FIG. 22C and thoracic/cervical regions of the spinal cord FIG. 22D, whole brain FIG. 22E, medulla FIG. 22F, pons FIG. 22G, spleen FIG. 22H, femur FIG. 22I and heart FIG. 22J of naive and EAE mice. High resolution ex vivo digital autoradiography of the spinal cord FIG. 22K and brain FIG. 22L supporting PET findings. Statistical analysis was performed using a 1-way ANOVA and Tukey's multiple comparison test. +vs. naive mice, #vs. KO EAE mice and *denotes direct comparison of groups (+p:S 0.05, ++p V 0.01, +++p:S 0.001, ++++p:S 0.0001, #p:S 0.05, ##p:S 0.01, ###p:S 0.001, ####p:S 0.0001, *P:S 0.05, **P=0.01). Data expressed as mean±SD.

FIGS. 23A-23L. TREMl-PET is a more sensitive tool for detecting neuroinflammation in EAE compared to the gold-standard TSPO-PET. TSPO-PET/CT imaging was performed 50-60 min following injection of [¹⁸F]GE-I 80 and tissues were harvested for ex vivo analysis FIG. 23A. Representative [¹⁸F]GE-180 images FIG. 23B. TSPO-PET quantification in cervical/thoracic spinal cord FIG. 23C lumbar spinal cord FIG. 23D and whole brain FIG. 23E of EAE (pre, low and high) and naive mice. Representative spinal cord and brain [¹⁸F]GE-180 autoradiography images (Niss I overlay) FIG. 23F. EAE-to-naive ratios of [⁶⁴Cu]TREMl-mAb and [¹⁸F]GE-180 signal using ex vivo autoradiography FIGS. 23G-231 and gamma counting FIGS. 23J-23L. Statistical analysis was performed using a 1-way ANOVA followed by Tukey's post-hoc test for PET quantification and t-tests for EAE-to-naive ratios. +vs.

FIGS. 24A-24J. TREMi+ cells are present in human MS white matter brain lesions. Demyelinated and myelinated regions of a MS white matter lesion were revealed with Luxol Fast Blue histochemical preparation FIG. 24A and Myelin Basic Protein immunohistochemical staining FIG. 24B in adjacent brain biopsy sections (drug- and steroid-naive tumefactive demyelinating MS). Severe axonal damage observed by staining of neurofilament FIG. 24C, which revealed transected degenerating axons and the formation of axonal bulbs (blue arrows). Similar pattern of axonal degeneration observed by Bielschowsky's Silver stain FIG. 24D. H&E staining revealed infiltration of immune cells in perivascular regions FIG. 24E, including a higher proportion of T lymphocytes FIGS. 24F and B lymphocytes FIG. 24G in perivascular regions and surrounding neural parenchyma. TREMi+ cells (red arrows) were observed in perivascular regions FIG. 24H, counterstained with hematoxylin). Compared to white matter of non-MS control tissue FIG. 24I, tumefactive MS white matter biopsy showed a high number of TREMi-positive cells FIG. 24J. Scale bar A-C, E-G, 1-J=50 μm, D, H=10 μm.

FIGS. 25A-25C. Flow cytometry gating strategy. Live single cells were differentiated into microglia (CD45intCDI Ib+), peripheral myeloid (CD45hiCDI Ib+) and lymphoid (CD4s+co1 I b−) cells. Separate gates were set for brain FIG. 25A, spinal cord FIG. 25B and spleen FIG. 25C. Myeloid cells were further differentiated into neutrophils (CD45hiCD 11 b+Ly6G+) and monocytes/macrophages/dendriticcells (DCs) (CD4511iCDI Ib+Ly6G} Levels of TREMi expression was investigated on all cell subtypes.

FIGS. 26A and 26B. TREMl expression on neutrophil and monocyte/macrophage/dendritic cell populations. Representative infiltrating TREM l+CD4511iCD 11 b+Ly6G+ neutrophils and CD45h.iCD 11 b+Ly6G-monocytes (Mo)/macrophages (M<I>)/dendritic cells (DCs) in the spinal cord of a WT EAE mouse FIG. 26A. Percentage of TREMl+ cells (% frequency of parent) in the spleen, spinal cord and brain of WT and Treml knockout (KO) EAE mice FIG. 26B. Statistical analysis was performed using a I-way ANOVA and Tukey's multiple comparison test. *vs. KO EAE. (**P:S 0.01, ***P:S 0.001. ****P:S 0.0001). Data expressed as mean±SD.

FIGS. 27A-27D. TREMl is not expressed on endothelial cells, astrocytes or neurons in EAE. Gating strategy to assess TREMl levels on endothelial cells (CD31+), astrocytes (CD4S−, ASCA2+), and neurons (CD4S−, CD90+) in spinal cord tissue form EAE mice FIG. 27A. Histogram of TREMl levels versus positive control beads FIG. 27B. TREMl+ cells as a proportion of all live single cells FIG. 27C and of parent cells FIG. 27D in spinal cord. Data expressed as mean±SD.

### DETAILED DESCRIPTION

Provided herein are methods and compositions for efficiently delivering one or more substance(s) to the brain of a subject. An antigen binding molecule is administered to the subject, wherein the antigen binding molecule binds an antigen in peripheral immune cells. Peripheral immune cells may be myeloid cells, NK cells, macrophages, and/or inflammatory cells that pass through the brain. The subject may require diagnosis of a disease or condition, such as multiple sclerosis, Alzheimer's disease, Huntington's disease, Parkinson's disease, epilepsy, brain tumor, stroke, amyotrophic lateral sclerosis, spinal cord and/or brain trauma, a disease or condition which would benefit from enzyme replacement therapy (“ERT”), a neurological disease, chronic inflammatory conditions, acute inflammatory conditions, or bacterial infection. The one or more antigen binding molecule(s) and/or one or more substance(s) may be one or more of antibody(s), biologic(s), peptide(s), small molecule(s), engineered protein scaffold(s), a nucleic acid, or a CRISPR-Cas9 molecule.

### Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 2nd ed. (1989) Cold Spring Harbor Laboratory Press, Cold Spring Harbor, NY. As appropriate, procedures involving the use of commercially available kits and reagents are generally carried out in accordance with manufacturer defined protocols and/or parameters unless otherwise noted.

As used herein, the singular forms “a,” “an,” and “the” include the plural referents unless the context clearly indicates otherwise.

The term “about” indicates and encompasses an indicated value and a range above and below that value. In certain embodiments, the term “about” indicates the designated value±10%, t 5%, or ±1%. In certain embodiments, the term “about” indicates the designated value±one standard deviation of that value.

The term “antigen” or “Ag” as used herein shall refer to a molecule or molecular structure that can be bound by a molecule, such as an antibody or a molecule on a B cell antigen receptor. Antigens are targeted by antibodies and can bind any suitable molecule. Antigens are usually proteins, peptides, and polysaccharides. Lipids and nucleic acids may also become antigens when combined with proteins and polysaccharides. Saccharides and lipids also qualify as antigens.

The term “antigen binding molecule” as used herein, refers to any molecule capable of binding to an antibody. The term antigen binding molecule may include, for example, without limitation, a protein, polypeptide, or molecular complex. An antigen binding molecule may comprise or consist of one or more of antibody(s), antibody fragment(s), biologic(s), peptide(s), small molecule(s), engineered protein scaffold(s), a nucleic acid, or a CRISPR-Cas9 molecule. An antigen binding molecule may include one or more complementary determining region (“CDR”) that alone or in combination with other molecules bind to a particular antigen.

As used herein, “TREM1,” “TREM-1,” “Triggering Receptor Expressed on Myeloid Cells 1,” “Triggering Receptor Expressed on Monocytes 1,” “CD354,” or “CD354 antigen” shall refer to a protein that in humans is encoded by the TREM1 gene.

As used herein, “TREM2,” “TREM-2,” “Triggering Receptor Expressed On Myeloid Cells 2,” “Triggering Receptor Expressed On Myeloid Cells 2a,” “Triggering Receptor Expressed On Monocytes 2,” “Trem2a,” “Trem2b,” “Trem2c,” and “PLOSLT shall refer to a protein that in humans is encoded by the TREM2 gene.

As used herein, “GPR84,” “G Protein-Coupled Receptor 84,” “EX33,” “G-Protein Coupled Receptor 84,” “Inflammation-Related G Protein-Coupled Receptor EX33,” Inflammation-Related G-Protein Coupled Receptor EX33,” and “GPCR4” shall refer to a protein that in humans is encoded by the GPR84 gene.

As used herein, a “toll-like receptor” or “TLR” shall refer to any one of a class of proteins that in humans plays a key role in the innate immune system. Toll-like receptors are single-pass membrane-spanning receptors usually expressed on sentinel cells such as macrophages and dendritic cells.

As used herein, “TSPO” or “translocator protein” shall refer to an 18 kDa protein mainly found in the outer mitochondrial membrane. In humans, TSPO is encoded by the TSPO gene. TSPO is considered the gold standard as a transport protein.

As used herein, a “nucleotide-binding oligomerization domain-like receptors,” “NOD-like receptors,” “NLRs,” and “nucleotide-binding leucine-rich repeat receptors” shall refer to intracellular sensors of pathogen-associated molecular patterns (PAMPs) that enter the cell via phagocytosis or pores and damage-associated molecular patterns (DAMPs) that are associated with cell stress. Nucleotide-binding oligomerization domain-like receptors are types of pattern recognition receptors (PRRs) and play key roles in the regulation of the innate immune response.

As used herein, “biologic,” “biopharmaceutical,” and “biological medical product” shall refer to a pharmaceutical drug product manufactured in, extracted from, or semisythesized from biological sources. A biologic can be, for example, without limitation, vaccines, blood, blood components, allergenics, somatic cells, gene therapies, tissues, recombinant therapeutic proteins, and living medicines used in cell therapy. Biologics can be composed of sugars, proteins, or nucleic acids or complex combinations of any of these substances. Biologics (or their precursors or components) are isolated from living sources-human, animal, plant, fungal, or microbial.

As used herein, “peptides” shall refer to short chains of amino acids between two and fifty amino acids, where the amino acids are linked by peptide bonds. Chains of fewer than 10 or fifteen amino acids are sometimes referred to as oligopeptides, such as dipeptides, tripeptides, and tetrapeptides. A polypeptide is a longer, continuous, unbranched peptide chain of up to approximately fifty amino acids.

A polypeptide that contains more than approximately fifty amino acids is known as a protein. Proteins consist of one or more polypeptide(s) arranged in a biologically functional way, often bound to ligands such as coenzymes and cofactors or to another protein or other macromolecule, such as DNA or RNA. All peptides except cyclic peptides have an N-terminal (amine) and a C-terminal (carboxyl group) residue at the end of the peptide. Peptides frequently have post-translational modifications such as phosphorylation, hydroxylation, sulfonation, palmitoylation, glycosylation, and disulfide formation.

As used herein, “small molecule” shall refer to a low molecular weight (<1000 daltons) organic compound that may regulate a biological process, with a size on the order of about 1 nm.

As used herein, “engineered protein scaffold” or “protein scaffold” refers to a protein, or part thereof, that has a defined three-dimensional structure when assembled and a capacity to support molecules or polypeptide domains in or on the structure.

As used herein, “CRISPR-Cas9 molecule” or “clustered regularly interspaced short palindromic repeats” refers to a family of DNA sequences found in the genomes of prokaryotic organisms such as bacteria and archaea. The CRISPR-Cas system is a prokaryotic immune system that confers resistance to foreign genetic elements such as those present within plasmids and phages and provides a form of acquired immunity. RNA harboring the spacer sequences helps Cas (CRISPR-associated) proteins recognize and cut foreign pathogenic DNA. CRISPR are found in approximately 50% of sequenced bacterial genomes and nearly 90% of sequenced archaea. CRISPR gene editing systems commonly utilize the cas9 gene.

As used herein, “nucleic acid” shall refer to the overall name for DNA and RNA and derivatives therefrom.

As used herein, “DNA” or “deoxyribonucleic acid” shall each refer to a nucleic acid containing the genetic instructions for functioning of organisms. DNA segments carrying information are called genes.

As used herein, “RNA” or “ribonucleic acid” shall refer to a polymeric molecule essential in various biological roles in coding, decoding, regulation, and expression of genes. Like DNA, RNA is assembled as a chain of nucleotides, but unlike DNA, RNA is found in nature as a single strand folded onto itself, rather than a paired double strand. Cellular organisms use messenger RNA (“mRNA”) to convey genetic information that directs synthesis of specific proteins.

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, PA. Briefly, each heavy chain typically comprises a heavy chain variable region (V_{H}) and a heavy chain constant region (C_{H}). The heavy chain constant region typically comprises three domains, CH1, CH2, and CH3. Each light chain typically comprises a light chain variable region (VL) and a light chain constant region. The light chain constant region typically comprises one domain, abbreviated CL.

The term “antibody” describes a type of immunoglobulin molecule and is used herein in its broadest sense. An antibody specifically includes intact antibodies (e.g., intact immunoglobulins) and antibody fragments.

The V_{H} and V_{L} regions may be further subdivided into regions of hypervariability (“hypervariable regions (HVRs);” also called “complementarity determining regions” (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2-FR3-CDR3-FR4. The CDRs are involved in antigen binding, and confer antigen specificity and binding affinity to the antibody. See Kabat et al., Sequences of Proteins of ImmunologicalInterest 5th ed. (1991) Public Health Service, National Institutes of Health, Bethesda, MD, incorporated by reference in its entirety.

The light chain from any vertebrate species can be assigned to one of two types, called kappa and lambda, based on the sequence of the constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“AHo” numbering scheme), each of which is incorporated by reference in its entirety.

Table 1 provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

Unless otherwise specified, the numbering scheme used for identification of a particular CDR herein is the Kabat numbering scheme. Variant and equivalent antibodies with a Chothia numbering scheme are intended to be within the scope of the invention.

TABLE 1
Residues in CDRs according to Kabat and Chothia numbering schemes.

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat | H31-H35B | H26-H32 or H34* |
| Numbering) | | |
| H1 (Chothia | H31-H35 | H26-H32 |
| Numbering) | | |
| H2 | H50-H65 | H52-H56 |
| H3 | H95-H102 | H95-H102 |

The C-terminus of CDR-H1, when numbered using the Kabat numbering convention, varies between H32 and H34, depending on the length of the CDR.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

An “antibody fragment” comprises a portion of an intact antibody, such as the antigen binding or variable region of an intact antibody. Antibody fragments include, for example, Fv fragments, Fab fragments, F(ab′)₂ fragments, Fab′ fragments, scFv (sFv) fragments, and scFv-Fc fragments.

“Fv” fragments comprise a non-covalently linked dimer of one heavy chain variable domain and one light chain variable domain.

“Fab” fragments comprise, in addition to the heavy and light chain variable domains, the constant domain of the light chain and the first constant domain (C_{H1}) of the heavy chain. Fab fragments may be generated, for example, by papain digestion of a full-length antibody.

“F(ab′)₂” fragments contain two Fab′ fragments joined, near the hinge region, by disulfide bonds. F(ab′)₂ fragments may be generated, for example, by pepsin digestion of an intact antibody. The F(ab′) fragments can be dissociated, for example, by treatment with β-mercaptoethanol.

“Single-chain Fv” or “sFv” or “scFv” antibody fragments comprise a V_{H} domain and a V_{L} domain in a single polypeptide chain. The V_{H} and V_{L} are generally linked by a peptide linker. See Plückthun A. (1994). Antibodies from Escherichia coli. In Rosenberg M. & Moore G. P. (Eds.), The Pharmacology of Monoclonal Antibodies vol. 113 (pp. 269-315). Springer-Verlag, New York, incorporated by reference in its entirety. “scFv-Fc” fragments comprise an scFv attached to an Fc domain. For example, an Fc domain may be attached to the C-terminal of the scFv. The Fc domain may follow the V_{H} or V_{L} depending on the orientation of the variable domains in the scFv (i.e., V_{H}-V_{L} or V_{L}-V_{H}). Any suitable Fc domain known in the art or described herein may be used.

The term “minibody” refers to an antibody fragment (such as one that contains a VL-VH-CH3) with bivalent binding to an antigen.

The term “monoclonal antibody” (mAb) refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “chimeric antibody” refers to an antibody in which a portion of the heavy and/or light chain is derived from a particular source or species, while the remainder of the heavy and/or light chain is derived from a different source or species.

“Humanized” forms of non-human antibodies are chimeric antibodies that contain minimal sequence derived from the non-human antibody. A humanized antibody is generally a human immunoglobulin (recipient antibody) in which residues from one or more CDR(s) are replaced by residues from one or more CDR(s) of a non-human antibody (donor antibody). The donor antibody can be any suitable non-human antibody, such as a mouse, rat, rabbit, chicken, llama, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

An “isolated antibody” is one that has been separated and/or recovered from a component of its natural environment. Components of the natural environment may include enzymes, hormones, and other proteinaceous or nonproteinaceous materials. In some embodiments, an isolated antibody is purified to a degree sufficient to obtain at least 15 residues of N-terminal or internal amino acid sequence, for example by use of a spinning cup sequenator. In some embodiments, an isolated antibody is purified to homogeneity by gel electrophoresis (e.g., SDS-PAGE) under reducing or non-reducing conditions, with detection by Coomassie blue or silver stain. An isolated antibody includes an antibody in situ within recombinant cells, since at least one component of the antibody's natural environment is not present. In some embodiments, an isolated antibody is prepared by at least one purification step.

With regard to the binding of an antibody to a target molecule, the terms “binding” or “binds to” a particular antigen (e.g., a polypeptide target) or an epitope on a particular antigen mean binding that is measurably different from a non-selective interaction. Binding can be measured, for example, by determining binding of a molecule compared to binding of a control molecule. Binding can also be determined by competition with a control molecule that is similar to the target, such as an excess of non-labeled target. In that case, binding is indicated if the binding of the labeled target to a probe is competitively inhibited by the excess non-labeled target.

Percent “identity” between a polypeptide sequence and a reference sequence is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, or CLUSTAL OMEGA software. Those skilled in the art can determine appropriate parameters for aligning sequences, including any algorithms needed to achieve maximal alignment over the full length of the sequences being compared.

A “conservative substitution” or a “conservative amino acid substitution,” refers to the substitution of one or more amino acid(s) with one or more chemically or functionally similar amino acid(s). Conservative substitution tables providing similar amino acids are well known in the art. Polypeptide sequences having such substitutions are known as “conservatively modified variants.” Such conservatively modified variants are in addition to and do not exclude polymorphic variants, interspecies homologs, and alleles. By way of example, the following groups of amino acids are considered conservative substitutions for one another.

| | |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |
| Alcohol Group-Containing Residues | S and T |
| Aliphatic Residues | I, L, V, and M |
| Cycloalkenyl-associated Residues | F, H, W, and Y |
| Hydrophobic Residues | A, C, F, G, H, I, L, M, |
| | T, V, W, and Y |
| Negatively Charged Residues | D and E |
| Polar Residues | C, D, E, H, K, N, Q, R, S, and T |
| Positively Charged Residues | H, K, and R |
| Small Residues | A, C, D, G, N, P, S, T, and V |
| Very Small Residues | A, G, and S |
| Residues Involved in Turn | A, C, D, E, G, H, K, N, Q, R, |
| Formation | S, P, and T |
| Flexible Residues | Q, T, K, S, G, P, D, E, and R |
| Group 1 | A, S, and T |
| Group 2 | D and E |
| Group 3 | N and Q |
| Group 4 | R and K |
| Group 5 | I, L, and M |
| Group 6 | F, Y, and W |
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2nd ed. (1993) W. H. Freeman & Co., New York, NY.

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (lie; I), leucine (Leu; L), lysine (Lys; K), methionine (Met; M), phenylalanine (Phe; F), proline (Pro; P), serine (Ser; S), threonine (Thr; T), tryptophan (Trp; V), tyrosine (Tyr; Y), and valine (Val; V).

As used herein, the term “peripheral immune cells” shall refer to immune cells that reside outside of the brain. During a neuroimmune response, sometimes peripheral immune cells are able to cross various blood or fluid brain barriers in order to respond to pathogens that have entered the brain. As the central nervous system is considered an immune-privileged organ due to the blood-brain barrier, there is a relatively low number of surveilling peripheral immune cells found within the brain parenchyma.

As used herein, “myeloid cells” or “myelogenous cells” shall refer to monocytes, macrophages, neutrophils, basophils, eosinophils, erythrocytes, and megakaryocytes and platelets. In some embodiments, myeloid cells shall refer to any cell of myeloid lineage.

As used herein, “NK cells,” “natural killer cells,” “large granular lymphocytes,” or “LGL” shall refer to a type of cytotoxic lymphocyte critical to the innate immune system. The role of NK cells is analogous to that of cytotoxic T cells in the vertebrate adaptive immune response.

As used herein, “macrophages” shall refer to a type of white blood cell of the immune system that engulfs and digests cellular debris, foreign substances, microbes, cancer cells, and anything else that does not have the type of proteins specific to healthy body cells on its surface in a process called phagocytosis. Macrophages are large phagocytes and are found in essentially all tissues, where they patrol for potential pathogens. They take various forms (with various names) throughout the body (e.g., histiocytes, Kupffer cells, alveolar macrophages, microglia, and others), but all are part of the mononuclear phagocyte system.

As used herein, “monocytes” shall refer to a type of leukocyte, or white blood cell. They are the largest type of leukocyte and can differentiate into macrophages and myeloid lineage dendritic cells. As a part of the vertebrate innate immune system monocytes also influence the process of adaptive immunity.

As used herein, “granulocytes,” “polymorphonuclear leukocytes,” “PMN,” “PML,” and “PMNL” shall refer to a category of white blood cells in the innate immune system characterized by the presence of granules in their cytoplasm. They have varying shapes of the nucleus and are usually divided into three segments. There are four types of granulocytes: basophils, eosinophils, neutrophils, and mast cells.

As used herein, “neutrophils” shall refer to the most abundant type of phagocyte found in the bloodstream, constituting 60% to 65% of the total circulating white blood cells and consisting of two subpopulations. Neutrophils stain a neutral pink on hematoxylin and eosin (H&E) histological or cytological preparations. They are formed of stem cells in bone marrow and divide into subpopulations of neutrophil-killers and neutrophil-cagers. They are short-lived and highly motile, or mobile, as they can enter parts of tissue where other cells/molecules cannot.

As used herein, “eosinophils,” “eosinophiles,” or “acidophils” shall refer to a variety of white blood cells which appear brick red after staining with eosin, a red dye, using the Romanowsky method. Eosinophils are largely responsible for combatting multicellular parasites and certain infections in vertebrates. They develop during hematopoiesis in the bone marrow before migrating to the blood, after which they are terminally differentiated and do not multiply.

Eosinophils are acid loving due to their large acidophilic cytoplasmic granules. Their small granules contain chemical mediators, such as eosinophil peroxidase, ribonuclease (RNase), deoxyribonuclease (DNase), lipase, plasminogen, and major basic protein. When these mediators are released during degranulation, they are highly toxic to both parasite and host tissues.

As used herein, “basophils” shall refer to a type of white blood cell that is susceptible to staining by basic dyes. Basophils are the least common type of granulocyte, representing about 0.5% to 1% of circulating white blood cells. Basophils are the largest type of granulocyte, however.

As used herein, “dendritic cells” shall refer to antigen-presenting cells (also known as accessory cells) of the mammalian immune system. Their main function is to process antigen material and present it on the cell surface to the T cells of the immune system. They act as messengers between the innate and adaptive immune systems.

As used herein, “B cells” or “B lymphocytes” shall refer to a type of white blood cell that functions in the humoral immunity component of the adaptive immune system by secreting antibodies. Additionally, B cells present antigen as professional antigen-presenting cells and secrete cytokines.

As used herein, “diagnosis” shall to identifying the nature or stage of an illness or other problem.

As used herein, “IV,” “intravenous,” or “intravenous route of administration” shall refer to fluid delivery directly into a vein. Intravenous can be used both for injections, using a syringe at higher pressures; as well as for infusions, typically using only the pressure supplied by gravity. Intravenous infusions are commonly referred to as drips.

As used herein, “intramuscular,” “intramuscular injection,” “IM injection,” or “IM” shall refer to injection of a substance directly into muscle.

As used herein, “subcutaneous” “subcutaneous injection,” “SC,” “SQ,” “sub-cu,” “sub-Q,” “SubQ,” or “subcut” shall refer to administration of a bolus into the subcutis, the layer of skin directly below the dermis and epidermis, collectively referred to as the cutis.

As used herein, “intraperitoneal” “intraperitoneal injection,” “IP injection,” or “IP” shall refer to injection of a substance into the peritoneum (body cavity).

As used herein, “intrathecal” “intrathecal administration,” or “intrathecal” shall refer to a route of administration for drugs via injection into the spinal canal or into the subarachnoid space so that it reaches the cerebrospinal fluid (CSF).

As used herein, “intravitreal” or “intravitreal administration” is a route of administration of a drug or other substance in which the substance is delivered to the vitreous humor of the eye.

As used herein, the term “subject” means a mammal or a human. In some embodiments subjects include, but are not limited to, monkeys, dogs, cats, mice, rats, cows, horses, camels, avians, goats, and sheep.

As used herein, “MRI,” “magnetic resonance imaging,” “nuclear magnetic resonance imaging,” or “NMR” shall refer to a medical imaging technique used in radiology to form pictures of the anatomy and the physiological processes of the body.

Antigen Binding Molecules and/or One or More Substance(s)

The current invention is drawn to methods and compositions for delivering one or more substance(s) to the brain in a subject in need thereof, wherein the one or more antigen binding molecule(s) that binds one or more antigen(s) in peripheral immune cells is administered to the subject. In some embodiments, the one or more antigen binding molecule(s) comprises or consists of one or more of antibody(s), antibody fragment(s), biologics, peptide(s), small molecule(s), engineered protein scaffold(s), a nucleic acid, or one or more CRISP-Cas9 molecule(s).

In some embodiments, the one or more antigen binding molecule(s) comprises or consists of antibodies and/or antibody fragments. In some embodiments, the antibody and/or antibody fragment comprises or consists of a monoclonal antibody or chimeric antibody. In some embodiments, the antibody or antibody fragment comprises or consists of a mouse antibody or antibody fragment. In some embodiments, the antibody or antibody fragment comprises or consists of a humanized antibody or antibody fragment. In some embodiment, the antibody or antibody fragment comprises or consists of a human antibody or antibody fragment. In some embodiments, the antibody or antibody fragment comprises or consists of an isolated antibody or antibody fragment.

In some embodiments, the antibody or antibody fragment comprises a binding domain that binds to one or more antigen(s). In some embodiments, the one or more antigen(s) comprises or consists of TREM1, TREM2, GPR84, a toll-like receptor, or a nucleotide-binding oligomerization domain-like receptor.

In some embodiments, the binding domain comprises a light chain. In some embodiments, the light chain is a kappa light chain. In some embodiments, the light chain is a lambda light chain.

In some embodiments, the binding domain comprises a heavy chain. In some embodiments, the heavy chain is an IgA. In some embodiments, the heavy chain is an IgD. In some embodiments, the heavy chain is an IgE. In some embodiments, the heavy chain is an IgG. In some embodiments, the heavy chain is an IgM. In some embodiments, the heavy chain is an IgG1. In some embodiments, the heavy chain is an IgG2. In some embodiments, the heavy chain is an IgG3. In some embodiments, the heavy chain is an IgG4. In some embodiments, the heavy chain is an IgA1. In some embodiments, the heavy chain is an IgA2.

In some embodiments, the binding domain is an antibody fragment. In some embodiments, the antibody fragment is an Fv fragment. In some embodiments, the antibody fragment is a Fab fragment. In some embodiments, the antibody fragment is a F(ab′)₂ fragment. In some embodiments, the antibody fragment is a Fab′ fragment. In some embodiments, the antibody fragment is an scFv (sFv) fragment. In some embodiments, the antibody fragment is an scFv-Fc fragment. In some embodiments, the antibody fragment is a minibody. In some embodiments, the antibody fragment is a single domain antibody.

In some embodiments, the binding domain is a chimeric antibody. In some embodiments, the binding domain is a humanized antibody. In some embodiments, the binding domain is a human antibody.

In some embodiments, the binding domain binds TREM1, TREM2, GPR84, a toll-like receptor, and/or a nucleotide-binding oligomerization domain-like receptor. In some embodiments, the binding domain binds TREM2. In some embodiments, the binding domain binds GPR84. In some embodiments, the binding domain binds a toll-like receptor. In some embodiments, the binding domain binds a nucleotide-binding oligomerization domain-like receptor.

In some embodiments, the binding domain binds TREM1 (See, for example, https://www.rndsystems.com/products/mouse-trem-1-antibody-174031_mab1187, which is incorporated by reference herein). In some embodiments, the binding domain has a certain percent identity to one or more sequence(s) of binding domains that bind TREM1. In some embodiments, the binding domain has a percent identity that is at least about 70%, at least about 75%, at least about 80%, at least about 85%, at least about 90%, or at least about 95% to one or more sequence(s) of binding domains that bind TREM1. In some embodiments, the binding domain has one or more conservative substitution(s) as compared to one or more sequence(s) of binding domains that bind TREM1.

In some embodiments, the one or more antigen binding molecule(s) comprises or consists of one or more peptide(s). In some embodiments, the one or more peptide(s) comprises a chain of amino acids between about two and about fifty amino acids. In some embodiments, the one or more peptide(s) comprises a chain of amino acids fewer than about 10 or fewer than about fifteen amino acids. In some embodiments, the one or more peptide(s) comprises or consists of one or more of a dipeptide(s), tripeptide(s), and/or tetrapeptide(s).

In some embodiments, the one or more antigen binding molecule(s) comprises or consists of one or more polypeptide(s). A polypeptide that contains more than approximately fifty amino acids is known as a protein. Proteins consist of one or more polypeptide(s) arranged in a biologically functional way, often bound to ligands such as coenzymes and cofactors or to another protein or other macromolecule, such as DNA or RNA. All peptides except cyclic peptides have an N-terminal (amine) and a C-terminal (carboxyl group) residue at the end of the peptide. Peptides frequently have post-translational modifications such as phosphorylation, hydroxylation, sulfonation, palmitoylation, glycosylation, and disulfide formation.

In some embodiments, the one or more antigen binding molecule(s) comprises or consists of one or more small molecule(s). Small molecules are organic compounds with a molecular weight that is usually less than about 1000 daltons. Small molecules may regulate a biological process. Larger structures such as nucleic acids and proteins and many polysaccharides are not small molecules, although their constituent monomers (ribo- or deoxyribonucleotides, amino acids, and monosaccharides, respectively) are often considered small molecules. Small molecules can have a variety of biological functions or applications, such as serving as cell signaling molecules or drugs or in many other roles. Small molecules may be natural or artificial.

In some embodiments, the one or more antigen binding molecules comprises of consist of one or more engineered protein scaffold(s). An engineered protein scaffold has a three-dimensional structure that when assembled has a capacity to support molecules and/or polypeptide domains in or on the structure.

In some embodiments, the one or more antigen binding molecule(s) comprises or consist of one or more nucleic acid(s). Nucleic acids are composed of nucleotides, monomers made of three components: a 5-carbon sugar, a phosphate group, and a nitrogenous base. If the sugar is the compound ribose, then the nucleic acid is RNA; if the sugar is deoxyribose or derived from deoxyribose, then the nucleic acid is DNA.

Nucleic acids are the most important of all biomolecules. They are found in all living things. They function to encode and store information of every living life form. They transmit and express that information from the interior of the cell. They also transmit information to the next generation of organism.

Information is ultimately encoded and conveyed via a sequence of nucleotides, the nucleic acid sequence. Strings of nucleotides are bounded to form helical backbones and assembled into chains of base-bases selected from canonical (and sometimes non-canonical) nucleobases. The nucleobases are adenine, cytosine, guanine, thymine, and uracil. Using amino acid and the process of protein synthesis, nucleic acid sequences store and transmit information, such as coded information in genes that allows one to express proteins.

In some embodiments, the one or more nucleic acids comprise one or more DNA molecules. DNA often consists of two long polymers of simple units called nucleotides, with backbones made of sugars and phosphate groups joined by ester bonds. These two strands run in opposite directions to each other and are, therefore, anti-parallel. The sequence of these four nucleobases along the backbone encodes the information.

Information is read using the genetic code, which specifies the sequence of the amino acids within proteins. The code is read by copying stretches of DNA into the related nucleic acid RNA in a process called transcription. Within cells, DNA is organized into long structures called chromosomes.

Eukaryotic organisms (animals, plants, fungi, and protists) store most of their DNA inside the cell nucleus and some of their DNA in organelles, such as mitochondria or chloroplasts. In contrast, prokaryotes (bacteria and archaea) store their DNA only in the cytoplasm. Within the chromosomes, chromatin proteins, such as histones, compact and organize DNA. These compact structures guide the interactions between DNA and other proteins, helping control which parts of the DNA are transcribed.

In some embodiments, the one or more nucleic acids comprise one or more RNA. Some RNA molecules play an active role within cells by catalyzing biological reactions, controlling gene expression, or sensing and communicating responses to cellular signals. One of the active processes is protein synthesis, a universal function in which RNA molecules direct the synthesis of proteins on ribosomes. This process uses transfer RNA (“mRNA”) molecules to deliver amino acids to the ribosome, where ribosomal (“rRNA”) then links amino acids together to form coded proteins.

In some embodiments, the one or more antigen binding molecule(s) comprises or consists of one or more CRISPR-Cas9 molecule(s). CRISPR-Cas9 sequences are derived from DNA fragments of bacteriophages that have previously infected the prokaryote. The sequences are used to detect and destroy DNA from similar bacteriophages during subsequent infections. Hence these sequences play a key role in the antiviral (i.e. anti-phage) defense system of prokaryotes.

The CRISPR-Cas system is a prokaryotic immune system that confers resistance to foreign genetic elements such as those present within plasmids and phages and provides a form of acquired immunity. RNA harboring the spacer sequences helps Cas (CRISPR-associated) proteins recognize and cut foreign pathogenic DNA. Other RNA-guided Cas proteins cut foreign RNA. CRISPR are found in approximately 50% of sequenced bacterial genomes and nearly 90% of sequenced archaea.

CRISPR gene editing systems commonly utilize the cas9 gene. This editing process has a wide variety of applications including basic biological research, development of products, and treatment of diseases (See, for example, Zhang F, Wen Y, Guo X (2014). CRISPR/Cas9 for genome editing: progress, implications and challenges. Human Molecular Genetics. 23); CRISPR-CAS9, TALENS and ZFNS—the battle in gene editing https://www.ptglab.com/news/blog/crispr-cas9-talens-and-zfns-the-battle-in-gene-editing; and Hsu P D, Lander E S, Zhang F (June 2014). Development and applications of CRISPR-Cas9 for genome engineering. Cell. 157 (6): 1262-1278, each of which is incorporated by reference herein in their entirety).

Antigens

The invention is drawn to one or more substance(s) to the brain in a subject in need thereof, comprising administering an antigen binding molecule to the subject, wherein the antigen binding molecule binds an antigen in peripheral immune cells. Any antigen present in peripheral immune cells would be suitable. For example, TREM1, TREM2, GPR84, a (inducible) toll-like receptor, or a nucleotide-binding oligomerization domain-like receptor would all be suitable antigens.

In some embodiments, the antigen comprises or consists of TREM1 (See for example the uniprot sequence listing for TREM1 at https://www.uniprot.org/uniprot/Q9NP99, which is incorporated by reference herein). In some embodiments, the antigen is a fragment or has a certain percent identity to TREM1. In some embodiments, the antigen has a percent identity that is at least about 70%, at least about 75%, at least about 80%, at least about 85%, at least about 90%, or at least about 95% to TREM1.

TREM 1 is a receptor belonging to the Ig superfamily and is expressed on myeloid cells. TREM1 amplifies neutrophil and monocyte-mediated inflammatory responses, such as those triggered by bacterial and fungal releases.

TREM1 is a highly specific biomarker of pro-inflammatory myeloid-driven immune responses in the well-established mouse model of systemic inflammation and LPS-induced sepsis. TREM1 expression is markedly elevated in the spleen, lungs, and brain after LPS challenge and is predominantly restricted to peripheral myeloid cells (i.e., dendritic cells, macrophages, monocytes, and neutrophils). Moreover, increased TREM1 binding was identified in these regions using [⁶⁴Cu]TREM1-mAb PET imaging, demonstrating the specificity and sensitivity of TREM1-PET to non-invasively visualize and track pro-inflammatory myeloid-driven immune responses in vivo.

TREM1-PET signal correlated with pro-inflammatory cytokine signatures and TREM1 K/O resulted in prolonged survival following LPS administration. (See, Bouchon, a, Facchetti, F., Weigand, M. a & Colonna, M. TREM-1 amplifies inflammation and is a crucial mediator of septic shock. Nature 410, 1103-1107 (2001); Schenk, M., Bouchon, A., Seibold, F. & Mueller, C. TREM-1-expressing intestinal macrophages crucially amplify chronic inflammation in experimental colitis and inflammatory bowel diseases. J. Clin. Invest. 117, 3097-3106 (2007); Poukoulidou, T. et al. TREM-1 expression on neutrophils and monocytes of septic patients: relation to the underlying infection and the implicated pathogen. BMC Infect Dis. 11, 309 (2011); Cohen, J. TREM-1 in sepsis. Lancet (London, England) 358, 776-8 (2001); and Nathan, C. & Ding, A. TREM-1: A new regulator of innate immunity in sepsis syndrome. Nat Med. 7, 530-532 (2001), each of which is hereby incorporated by reference in its entirety).

TREM1 may be useful in sepsis (See, for example, Gibot, S. et al. Plasma level of a triggering receptor expressed on myeloid cells-1: Its diagnostic accuracy in patients with suspected sepsis. Ann. Intern. Med. 141, 9-15+1 (2004); which is incorporated by reference herein in its entirety) and also in a broad range of chronic inflammatory diseases including inflammatory bowel disease (IBD), arthritis, and cancer. TREM1 is also a major contributor to cerebral injury following stroke (See, Liu, Q. et al. Peripheral TREM1 responses to brain and intestinal immunogens amplify stroke severity. Nat. Immunol. (2019), which is incorporated by reference in its entirety herein), indicating a role for TREM1 in the pathophysiology of neurological disease.

Whole-body TREM1-PET imaging has not only demonstrated increased peripheral myeloid cell infiltration into ischemic brain tissue but also revealed increased peripheral innate immune responses in the spleen and intestines of stroked mice. Further investigation of TREM1 expression on intestinal macrophages showed that it plays a role in gut permeability and bacterial translocation, highlighting the advantages of TREM1-PET as a tool to characterize innate immune responses in the whole-body, which may not be revealed when using traditional ex vivo approaches.

In some embodiments, the antigen comprises or consists of TREM2. The TREM2 protein functions in immune response and may be involved in chronic inflammation by triggering the production of constitutive inflammatory cytokines. TREM2 binds phospholipids (preferably anionic lipids) such as phosphatidylserine, phosphatidylethanolamine, phosphatidylglycerol, and sphingomyelin.

TREM2 regulates microglial proliferation by acting as an upstream regulator of the Wht/beta-catenin signaling cascade. TREM2 is required for microglial phagocytosis of apoptotic neurons and also required for microglial activation and phagocytosis of myelin debris after neuronal injury and of neuronal synapses during synapse elimination in the developing brain. TREM2 regulates microglial chemotaxis and process outgrowth and also the microglial response to oxidative stress and lipopolysaccharide. TREM2 suppresses PI3K and NF-kappa-B signaling in response to lipopolysaccharide, thus promoting phagocytosis, suppressing pro-inflammatory cytokine and nitric oxide production, inhibiting apoptosis, and increasing expression of IL10 and TGFB. During oxidative stress, TREM2 promotes anti-apoptotic NF-kappa-B signaling and ERK signaling.

TREM2 plays a role in microglial MTOR activation. TREM2 regulates age-related changes in microglial numbers. TREM2 triggers activation of immune responses in macrophages and dendritic cells. TREM2 mediates cytokine-induced formation of multinucleated giant cells which are formed by the fusion of macrophages. In dendritic cells, TREM2 mediates up-regulation of chemokine receptor CCR7 and dendritic cell maturation and survival. TREM2 is involved in the positive regulation of osteoclast differentiation.

In some embodiments, the antigen comprises or consists of GPR84. GPR84 is a member of the metabolic G protein-coupled receptor family and its expression has been described predominantly in immune cells. GPR84 activation is involved in the inflammatory response, but the mechanisms by which it modulates inflammation have been incompletely described.

In some embodiments, the antigen comprises or consists of one or more toll-like receptor(s). Toll-like receptors are single-pass membrane-spanning receptors usually expressed on sentinel cells such as macrophages and dendritic cells. Toll-like receptors usually recognize structurally conserved molecules derived from microbes. Once microbes have breached physical barriers such as the skin or intestinal tract mucosa, they are recognized by TLRs, which activate immune cell responses. Toll-like receptors include TLR1, TLR2, TLR3, TLR4, TLR5, TLR6, TLR7, TLR8, TLR9, TLR10, TLR11, TLR12, and TLR13, though the last three are not found in humans.

In some embodiments, the antigen comprises or consists of one or more nucleotide-binding oligomerization domain-like receptor(s). Nucleotide-binding oligomerization domain-like receptors are types of pattern recognition receptors (PRRs) and play key roles in the regulation of the innate immune response. Nucleotide-binding oligomerization domain-like receptors can cooperate with toll-like receptors and regulate inflammatory and apoptotic response.

Nucleotide-binding oligomerization domain-like receptors are found in lymphocytes, macrophages, and dendritic cells. They are also found in non-immune cells, for example, those in the epithelium. Nucleotide-binding oligomerization domain-like receptors are highly conserved through evolution and their homologs have been discovered in many different animal species, as well as the plant kingdom.

NLRs contain a NACHT (NOD or NBD—nucleotide-binding domain) domain, which is common to all NLRs. Most NLRs also have a C-terminal leucine-rich repeat (LRR) and a variable N-terminal interaction domain. NACHT domain mediates ATP-dependent self-oligomerization and LRR senses the presence of ligand. N-terminal domain is responsible for homotypic protein-protein interaction and can consist of caspase recruitment domain (CARD), pyrin domain (PYD), acidic transactivating domain, or baculovirus inhibitor repeats (BIRs).

Well-described receptors include NOD1 and NOD2. The recognition of their ligands recruits oligomerization of NACHT domain and CARD-CARD interaction with CARD-containing serine-threonine kinase RIP2 which leads to activation of RIP2. RIP2 mediates the recruitment of kinase TAK1 which phosphorylates and activates IκB kinase. The activation of IκB kinase results in the phosphorylation of inhibitor IκB which releases NF-κB and its nuclear translocation. NF-κB then activates expression of inflammatory cytokines.

NOD1 and NOD2 recognize peptidoglycan motifs from bacterial cell which consists of N-acetylglucosomine and N-acetylmuramic acid. These sugar chains are cross-linked by peptide chains that can be sensed by NODs. NOD1 recognizes a molecule called meso-diaminopimelic acid (meso-DAP) mostly found in Gram-negative bacteria (for example Helicobacter pylori, Pseudomanas aeruginosa). NOD2 proteins can sense intracellular muramyl dipeptide (MDP), typical for bacteria such as Steptococcus pneumoniae or Mycobacterium tuberculosis.

NLRPs and IPAF subfamilies of NLRs and are involved in the formation of the inflammasome. The best characterized inflammasome is NLRP3, the activation of which through PAMPs or DAMPs leads to the oligomerization. The pyrin domain of NLRs binds to an adaptor protein ASC (PYCARD) via PYD-PYD interaction. ASC contains PYD and CARD domain and links the NLRs to an inactive form of caspase 1 through the CARD domain. All these protein-protein interactions form a complex called the inflammasome.

One skilled in the art would recognize other antigens in peripheral immune cells (See, Taylor P. R. X et al. Macrophage Receptors and Immune Recognition. Annu. Rev. Immunol. 2005. 23: 901-44, which is incorporated by reference in its entirety herein). For example, without limitation, in some embodiments, the antigen comprises or consists of one or more of SR-A, CD36, CD14, CD11b, TLT1 (TREM-like inhibitor), CD200 receptor, SIRPa (aka CD172a), M-CSF receptor, Siglec-1, Siglec-3, PIRB, CD69, Mannose Receptor, CR3, CR4, Axl, Mer, EMR1, EMR2, EMR3, EMR4, CD14, Ly49Q, MICL (myeloid inhibitor C-type lectin Receptor), CLEC-1/2, KLRF1 (Killer cell lectin-like receptor 1), and/or MDL-1 (myeloid DAP12-associating Lectin-1).

Peripheral Immune Cells

The methods and compositions of delivering one or more substances to a brain of a subject in need comprising administering an antigen binding molecule to the brain of the subject in need, wherein the antigen binding molecule binds an antigen in peripheral immune cells can be applied to any peripheral immune cells that pass through the brain. Antigen binding molecules are pulled into the brain since peripheral immune cells are very good at crossing the BBB in certain contexts. In some embodiments, the peripheral immune cells comprise or consists of one or more of myeloid cell(s), NK cell(s), macrophage(s), monocyte(s), granulocyte(s), dendritic cell(s), and/or inflammatory cells that pass through the brain. In some embodiments, the granulocytes comprise or consist of one or more of neutrophil(s), eosinophil(s) and basophil(s).

In some embodiments, the peripheral immune cells comprise or consist of one or more myeloid cell(s). Myeloid is the general term that refers to monocytes, macrophages, neutrophils, basophils, eosinophils, erythrocytes, and megakaryocytes and platelets. In some embodiments, the one or more myeloid cell(s) comprises or consists of one or more erythrocyte(s). In some embodiments, the one or more myeloid cell(s) comprises or consists of one or more megakaryocyte(s). In some embodiments, the one or more myeloid cells comprises or consists of one or more platelet(s).

In some embodiments, the peripheral immune cells comprise or consist of one or more NK cell(s). NK cells provide rapid responses to virus-infected cells acting at around 3 days after infection. NK cells also respond to tumor formation.

Typically, immune cells detect the major histocompatibility complex (MHC) presented on infected cell surfaces, triggering cytokine release, causing the death of the infected cell by lysis or apoptosis. NK cells are unique, however, as they have the ability to recognize and kill stressed cells in the absence of antibodies and MHC, allowing for a much faster immune reaction.

NK cells were named “natural killers” because of the notion that they do not require activation to kill cells that are missing “self” markers. This role is especially important because harmful cells that are missing MHC I markers cannot be detected and destroyed by other immune cells, such as T lymphocyte cells.

NK cells can be identified by the presence of CD56 and the absence of CD3 (CD56⁺,CD3⁻). NK cells differentiate and mature in the bone marrow, lymph nodes, spleen, tonsils, and thymus. NK cells differ from natural killer T cells (NKTs) phenotypically, by origin and by respective effector functions.

Often, NKT cell activity promotes NK cell activity by secreting interferon gamma. In contrast to natural killer T cells, NK cells do not express T cell receptors or pan T marker CD3 or surface immunoglobulin (Ig) B cell receptors.

In addition to natural killer cells being effectors of innate immunity, both activating and inhibitory NK cell receptors play important functional roles, including self-tolerance and the sustaining of NK cell activity. NK cells also play a role in the adaptive immune response.

NK cells are cytotoxic. Small granules in their cytoplasm contain proteins such as perforin and proteases. Upon release in close proximity to a cell slated for killing, perforin forms pores in the cell membrane of the target cell, creating an aqueous channel through which the granzymes and associated molecules can enter, inducing either apoptosis or osmotic cell lysis.

Cytokines play a crucial role in NK cell activation. Cytokines serve to signal to the NK cell the presence of viral pathogens in the affected area. Cytokines involved in NK activation include IL-23, IL-15, IL-18, IL-2, and CCLS.

NK cells are activated in response to interferons or macrophage-derived cytokines. They serve to contain viral infections while the adaptive immune response generates antigen-specific cytotoxic T cells that can clear the infection. NK cells work to control viral infections by secreting IFNγ and TNFα. IFNγ activates macrophages for phagocytosis and lysis and TNFα acts to promote direct NK tumor cell killing. Natural killer cells often lack antigen-specific cell surface receptors, so are part of innate immunity, i.e. able to react immediately with no prior exposure to the pathogen. NKs can be seen to play a role in tumor immunosurveillance by directly inducing the death of tumor cells, even in the absence of surface adhesion molecules and antigenic peptides. The role of NK cells is critical to immune success particularly because T cells are unable to recognize pathogens in the absence of surface antigens. Tumor cell detection results in activation of NK cells and consequent cytokine production and release.

In some embodiments, the peripheral immune cells comprise or consist of one or more macrophage(s). Macrophages are large phagocytes and are found in essentially all tissues, where they patrol for potential pathogens. They take various forms (with various names) throughout the body (e.g., histiocytes, Kupffer cells, alveolar macrophages, microglia, and others), but all are part of the mononuclear phagocyte system.

Besides phagocytosis, they play a critical role in innate immunity and also help initiate specific defense mechanisms of adaptive immunity by recruiting other immune cells such as lymphocytes. They are important as antigen presenters to T cells.

Beyond increasing inflammation and stimulating the immune system, macrophages also play an important anti-inflammatory role and can decrease immune reactions through the release of cytokines. Macrophages that encourage inflammation are called M1 macrophages, whereas those that decrease inflammation and encourage tissue repair are called M2 macrophages. This difference is reflected in their metabolism; M1 macrophages have the unique ability to metabolize arginine to the “killer” molecule nitric oxide, whereas M2 macrophages have the unique ability to metabolize arginine to the “repair” molecule omithine.

Human macrophages are about 21 micrometers (0.00083 in) in diameter and are produced by the differentiation of monocytes in tissues. They can be identified using flow cytometry and immunohistochemical staining through expression of proteins, such as CD14, CD40, CD11b, CD64, F4/80, (mice)/EMR1 (human), lysozyme M, MAC-1/MAC-3, and CD68.

Macrophages are phagocytes and are highly specialized in removal of dying or dead cells and cellular debris. This role is important in chronic inflammation, as the early stages of inflammation are dominated by neutrophils, which are ingested by macrophages if they come of age. The neutrophils are at first attracted to a site, where they perform their function and die, before they are phagocytized by the macrophages. When at the site, the first wave of neutrophils, after the process of aging and after the first 48 hours, stimulate the appearance of the macrophages whereby these macrophages will then ingest the aged neutrophils.

The removal of dying cells is, to a greater extent, handled by fixed macrophages, which will stay at strategic locations such as the lungs, liver, neural tissue, bone, spleen, and connective tissue, ingesting foreign materials such as pathogens and recruiting additional macrophages if needed.

Macrophages are responsible for protecting tissues from foreign substances, but are also suspected to be important in the formation of important organs like the heart and brain. They are cells that possess a large smooth nucleus, a large area of cytoplasm, and many internal vesicles for processing foreign material.

In some embodiments, the peripheral immune cells comprise or consist of one or more monocyte(s). Monocytes are amoeboid in appearance and have nongranulated cytoplasm. Containing unilobar nuclei, monocytes are one of the types of mononuclear leukocytes which shelter azurophil granules. The archetypal geometry of the monocyte nucleus is ellipsoidal; metaphorically bean-shaped or kidney-shaped, although the most significant distinction is that the nuclear envelope is not hyperbolically furcated into lobes.

Monocytes compose 2% to 10% of all leukocytes in the human body and serve multiple roles in immune function. Such roles include replenishing resident macrophages under normal conditions; migration within approximately 8-12 hours in response to inflammation signals from sites of infection in the tissues; and differentiation into macrophages or dendritic cells to effect an immune response. In an adult human, half of the monocytes are stored in the spleen. These change into macrophages after entering into appropriate tissue spaces and can transform into foam cells in endothelium.

Monocytes are produced by the bone marrow from precursors called monoblasts, bipotent cells that differentiate from hematopoietic stem cells. Monocytes circulate in the bloodstream for about one to three days and then typically move into tissues throughout the body where they differentiate into macrophages and dendritic cells. They constitute between three and eight percent of the leukocytes in the blood.

Monocytes and their macrophage and dendritic cell progeny serve three main functions in the immune system. These are phagocytosis, antigen presentation, and cytokine production. Monocytes can perform phagocytosis using intermediary (opsonizing) proteins such as antibodies or complement that coat the pathogen, as well as by binding to the microbe directly via pattern-recognition receptors that recognize pathogens. Monocytes are also capable of killing infected host cells via antibody-dependent cell-mediated cytoxicity. Vacuolization may be present in a cell that has recently phagocytized foreign matter.

Many factors produced by other cells can regulate the chemotaxis and other functions of monocytes. These factors include most particularly chemokines, such as monocyte chemotactic protein-1 (CCL2) and monocyte chemotactic protein-3 (CCL7); certain arachidonic acid metabolites such as Leukotriene B4 and members of the 5-Hydroxycosaetraenoic acid and 5-oxo-eicosatetraenoic family of OXE1 receptor agonists (e.g., 5-HETE and 5-oxo-ETE); and N-Formylmethionone leucyl-phenylalanine and other N-formylated oligopeptides which are made by bacteria and activate the formyl peptide receptor 1.

Microbial fragments that remain after such digestion can serve as antigens. The fragments can be incorporated into MHC molecules and then trafficked to the cell surface of monocytes (and macrophages and dendritic cells). This process is called antigen presentation and it leads to activation of T lymphocytes, which then mount a specific immune response against the antigen.

Other microbial products can directly activate monocytes and this leads to production of pro-inflammatory and, with some delay, of anti-inflammatory cytokines. Typical cytokines produced by monocytes are TNF, IL-1, and IL-2.

In some embodiments, the peripheral immune cells comprise or consist of one or more granulocyte(s). Granulocytes are white blood cells in the innate immune system characterized by the presence of granules in their cytoplasm. They have varying shapes of the nucleus and their nucleus is usually divided into three segments. There are four types of granulocytes: basophils, eosinophils, neutrophils, and mast cells. In some embodiments, the granulocytes comprise or consist of one or more neutrophil(s). Neutrophils are phagocytic and are normally found in the bloodstream. During the beginning phase of inflammation, particularly as a result of bacterial infection, environmental exposure, and some cancers, neutrophils are one of the first responders of inflammatory cells to migrate toward the site of inflammation. Neutrophils migrate through the blood vessels and then through interstitial tissue, following chemical signals such as IL-8, C5a, fMLP, Leukotriene B4, and H₂O₂ during chemotaxis.

Neutrophils have three strategies for directly attacking micro-organisms: phagocytosis, release of soluble anti-microbial (including granule proteins), and generation of neutrophil extracellular traps (NETs). Neutrophils can also secrete products that stimulate monocytes and macrophages; these secretions increase phagocytosis and the formation of reactive oxygen compounds involved in intracellular killing.

In some embodiments, the granulocytes comprises or consist of one or more eosinophil(s). Eosinophils are largely responsible for combatting multicellular parasites and certain infections in vertebrates. They develop during hematopoiesis in the bone marrow before migrating to the blood, after which they are terminally differentiated and do not multiply.

Eosinophils are acid loving due to their large acidophilic cytoplasmic granules. Their small granules contain chemical mediators, such as eosinophil peroxidase, ribonuclease (RNase), deoxyribonuclease (DNase), lipase, plasminogen, and major basic protein. When these mediators are release during degranulation, they are highly toxic to both parasite and host tissues.

In normal individuals, eosinophils make up about 1-3% of white blood cells, and are about 12-17 micrometers in size with bilobed nuclei. While they are released into the bloodstream as neutrophils are, eosinophils reside in tissue. They are found in the medulla and the junction between the cortex and medulla of the thymus and in the lower gastrointestinal tract, ovaries, uterus, spleen, and lymph nodes. They are not found in the lungs, skin, esophagus, or other internal organs.

Eosinophils persist in the circulation for 8-12 hours and can survive in tissue for an additional 8-12 days in the absence of stimulation. Eosinophils are unique granulocytes as they have the capacity to survive for extended periods of time after their maturation.

Following activation, eosinophils effector functions include production of a) cationic granule proteins and their release by degranulation; b) reactive oxygen species such as hypobromite, superoxide, and peroxide; c) lipid mediators, like eicosanoids from the leukotriene and prostaglandin families, d) enzymes such as elastase; e) growth factors such as TGF beta, VEGF, and PDGF; and f) cytokines such as IL-1, IL-2, IL-4, IL-5, IL6, IL-8, IL-13, and TNF-alpha. Major basic protein, eosinophil peroxidase, and eosinophil cationic protein are toxic to many tissues.

Eosinophil cationic protein and eosinophil-derived neurotoxin are ribonucleases with antiviral activity. Eosinophil cationic protein creates toxic pores in the membranes of target cells, allowing potential entry of other cytotoxic molecules to the cell, can inhibit proliferation of T cells, suppress antibody production by B cells, induce degranulation by mast cells, and stimulate fibroblast cells to secrete mucus and glycosaminoglycan. Eosinophil peroxidase forms reactive oxygen species and reactive nitrogen intermediates that promote oxidative stress in the target, causing cell death by apoptosis and necrosis.

There are also eosinophils that play a role in fighting viral infections, which is evident from the abundance of RNases they contain within their granules, and in fibrin removal during inflammation. Eosinophils are responsible for tissue damage and inflammation in many diseases, including asthma. High levels of IL-5 has been observed to up regulate the expression of adhesion molecules, which then facilitate the adhesion of eosinophils to endothelial cells, thereby causing inflammation and tissue damage. An accumulation of eosinophils in the nasal mucosa is considered a major diagnostic criterion for nasal allergies.

In some embodiments, the granulocytes comprises or consist of one or more basophil(s). Basophils are white blood cells that are susceptible to staining by basic dyes. Basophils are the least common type of granulocyte, representing about 0.5% to 1% of circulating white blood cells. Basophils are the largest type of granulocyte, however.

Basophils are responsible for inflammatory reactions during immune responses, as well as in the formation of acute and chronic allergic diseases, including anaphylaxis, asthma, atopic dermatitis, and hay fever. They also produce compounds that co-ordinate immune responses, including histamine and serotonin that induce inflammation and heparin that prevents blood clotting.

Basophils arise and mature in bone marrow. When activated, basophils degranulate to release histamine, proteogycans such as heparin and chondroitin, and proteolytic enzymes such as elastase and lysophospholiopase. Basophils also secrete lipid mediators like leukotrienes (LTD-4) and several cytokines. Histamine and proteoglycans are pre-stored in the cell's granules while the other secreted substances are newly generated. Each of these substances contributes to inflammation.

In some embodiments, the peripheral immune cells comprise or consist of one or more dendritic cell(s). Dendritic cells main function is to process antigen material and present it on the cell surface to the T cells of the immune system. They act as messengers between the innate and adaptive immune systems.

Dendritic cells are present in those tissues that are in contact with the external environment, such as the skin (where there is a specialized dendritic cell type called the Langerhands cell) and the inner lining of the nose, lungs, stomach, and intestines. They can also be found in an immature state in the blood.

Once activated, dendritic cells migrate to the lymph nodes where they interact with T cells and B cells to initiate and shape the adaptive immune response. Every helper T-cell is specific to one particular antigen. Only professional antigen-presenting cells (macrophages, B lymphocytes, and dendritic cells) are able to activate a resting helper T-cell when the matching antigen is presented. However, in non-lymphoid organs, macrophages and B cells can only activate memory T cells whereas dendritic cells can activate both memory and naive T cells and are the most potent of all the antigen-presenting cells.

In the lymph node and secondary lymphoid organs, all three cell types can activate naive T cells. Whereas mature dendritic cells are able to activate antigen-specific naive CD8⁺ T cells, the formation of CD8⁺ memory T cells requires the interaction of dendritic cells with CD4⁺ helper T cells. This help from CD4⁺ T cells additionally activates the matured dendritic cells and licenses them to efficiently induce CD8⁺ memory T cells, which are also able to be expanded a second time. For this activation of dendritic cells, concurrent interaction of all three cell types, namely CD4⁺ T helper cells, CD8⁺ T cells, and dendritic cells, seems to be required.

Administration and Composition(s)

The methods and compositions of the current invention may be administered by any suitable method. Such methods include, for example, without limitation, IV, intramuscular, subcutaneous, intraperitoneal, intravitreal, or intrathecal administration.

In some embodiments, administration comprises or consists of IV administration. Intravenous administration can be used both for injections, using a syringe at higher pressures; as well as for infusions, typically using only the pressure supplied by gravity. Intravenous infusions are commonly referred to as drips. Intravenous administration is the fastest way to deliver medications and fluid replacement throughout the body, because they are introduced directly into the circulation.

A continuous infusion may be used to correct fluid and electrolyte imbalances, or when it is desirable to have a constant blood concentration of a medication over time. Continuous infusions are used where the variation in concentration that arises from gaps in administration would be undesirable. They may also be used instead of intermittent bolus injections for the same reason.

Infusions can also be intermittent, in which case the medication is administered over a period of time, then stopped, and this is later repeated. Intermittent infusion may be used when there are concerns about the stability of medicine in solution for long periods of time (as is common with continuous infusions), or to enable the administration of medicines which would be incompatible if administered at the same time in the same IV line.

Any additional medication to be administered IV at the same time as an infusion may be connected to the primary tubing; this is termed a secondary IV, or IV piggyback. This prevents the need to use multiple IV access lines on the same person. When administering a secondary IV medication, the primary bag is held lower than the secondary bag so that the secondary medication can flow into the primary tubing, rather than fluid from the primary bag flowing into the secondary tubing. The fluid from the primary bag is needed to help flush any remaining medication from the secondary IV from the tubing into the patient.

Some medications are administered as a bolus dose, called IV push. A syringe containing the medication is connected to an access port in the primary tubing and the medication is administered through the port. A bolus dose may be administered rapidly or may also be administered over the course of a few minutes, depending on the medication. In some cases, a bolus of non-medicated solution is administered after the medication to push the medicine into the bloodstream, termed a flush.

A standard IV infusion set consists of a pre-filled, sterile container (glass bottle, plastic bottle, or plastic bag) of fluids with an attachment that allows the fluid to flow one drop at a time, making it easy to see the flow rate and reducing the risk of air bubbles. These kits may also contain a sterile tube, a clamp to regulate flow, a connector to attach the access device, and devices to enable piggybacking. Many systems of administration employ a drip chamber, which prevents air from entering the bloodstream (air embolism), and allows an estimation of flow rate.

An IV administration may consist only of a bag of fluid hanging on a pole above the height of the person to whom medication is being administered. In this way, gravity will cause the fluid to flow into the IV line and the person's vein. In such “gravity” IVs, it is not possible to precisely control the rate of administration.

Alternatively, an infusion pump may be used to allow precise control over the flow rate and total amount delivered. A pump will be programmed based on the number and size of infusions being administered to ensure all medicine is fully administered without allowing the access line to run dry. Pumps are primarily utilized when a constant flow rate is important or where changes in rate of administration would have consequences.

The simplest form of intravenous access is by passing a hollow needle through the skin directly into the vein. This needle can be connected directly to a syringe (used either to withdraw blood or deliver its contents into the bloodstream) or may be connected to a length of tubing and thence whichever collection or infusion system is desired.

A peripheral cannula is the most common intravenous access method utilized in hospitals. The most convenient site is often the arm, especially the veins on the back of the hand, or the median cubital vein at the elbow, but any identifiable vein can be used. Often it is necessary to use a tourniquet which restricts the venous drainage of the limb and makes the vein bulge.

Once the needle is in place, it is common to draw back slightly on the syringe to aspirate blood, thus verifying that the needle is really in a vein. The tourniquet should be removed before injecting to prevent extravasation of the medication. The part of the catheter that remains outside the skin is called the connecting hub; it can be connected to a syringe or an intravenous infusion line, or capped with a heplock or saline lock, a needleless connection filled with a small amount of heparin or saline solution to prevent clotting, between uses of the catheter. Ported cannula have an injection port on the top that is often used to administer medicine.

In some embodiments, administration comprises or consists of intramuscular administration. Muscles have larger and more numerous blood vessels than subcutaneous tissue. Intramuscular injections usually have faster rates of absorption than subcutaneous or intradermal injections. The volume of injection is often limited to 2-5 milliliters, depending on injection site.

Possible sites for IM injection include: deltoid, dorsoguteal, rectus femoris, vastus laterialis, and ventrogluteal muscles. Sites that are bruised, tender, red, swollen, inflamed or scarred are avoided.

To perform an IM injection, the selected site is cleansed with an antimicrobial and is allowed to dry. It is injected with the dominant hand using a quick, darting motion perpendicular to the patient's body at an angle between 72 and 90 degrees, as a faster injection is less painful. The needle is then stabilized with the non-dominant hand while the dominant hand slides to the plunger to slowly instill the medication, as a rapid injection causes more discomfort. The needle is withdrawn at the same angle inserted. This is to ensure that the medication does not leak back along the needle track. Gentle pressure is applied with a gauze but the site is not massaged to prevent forcing the medication into subcutaneous tissue.

In some embodiments, administration comprises or consists of subcutaneous administration. Subcutaneous tissue has few blood vessels and so drugs injected subcutaneously are for slow, sustained rates of absorption. Sites of injection may include the outer area of the upper arm, the abdomen, from the rib margin to the iliac crest and avoiding a 2-inch circle around the navel, the front of the thigh, midway to the outer side, 4 inches below the top of the thigh to 4 inches above the knee, the upper back, and the upper area of the buttock just behind the hip bone. Subcutaneous injections are inserted at 45 to 90 degree angles, depending on amount of subcutaneous tissue present and length of needle—a shorter, 3/8″ needle is usually inserted 90 degrees and a 5/8″ needle is usually inserted at 45 degrees.

In some embodiments, administration comprises or consists of intraperitoneal administration. Intraperitoneal administration may be preferred when large amounts of blood replacement fluids are needed or when low blood pressure or other problems prevent the use of a suitable blood vessel for intravenous injection.

In some embodiments, administration comprises or consists of intrathecaladministration. Intrathecal administration is often used as away to avoid the BBB. Drugs given by the intrathecal route often have to be compounded specially by a pharmacist or technician because they cannot contain any preservative or other potentially harmful inactive ingredients that are sometimes found in standard injectable drug preparations.

In some embodiments, administration comprises or consists of intravitreal administration. Intravitreal administration is a route of administration where a drug or other substance is delivered to the vitreous humor of the eye.

The antigen binding compositions suitable for the invention may comprise or consist of additional substances. For example, the composition may comprise one or more excipient(s). Any suitable excipient may be used, and one of ordinary skill in the art is capable of selecting suitable excipients. Additional excipients include, for example, those described in the Handbook of Pharmaceutical Excipients, Rowe et al. (Eds.) 6th Ed. (2009), incorporated by reference herein in its entirety.

Further encompassed herein are anhydrous compositions and dosage forms comprising an antibody. Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

Nucleic Acids, Vectors, Hosts, and Cell Lines

A third aspect provides a nucleic acid encoding any of the compositions or the antigen binding molecules set forth herein. In some embodiments, the nucleic acid comprises a vector. Some embodiments provide a host transformed with the vector. Some embodiments provide a method for the production of one or more composition(s) or the antigen binding molecules comprising the steps of expressing any of the nucleic acid provided herein in a prokaryotic or eukaryotic host cell and recovering the antigen binding molecules from the cell or the cell culture supernatant.

For recombinant production of the compositions or the antigen binding molecules, the nucleic acid encoding it may be isolated and inserted into a replicable vector for further cloning (i.e., amplification of the DNA) or expression. In some aspects, the nucleic acid may be produced by homologous recombination, for example as described in U.S. Pat. No. 5,204,244.

Many different vectors are known in the art. The vector components generally include, but are not limited to, one or more of the following: a signal sequence, an origin of replication, one or more marker gene(s), an enhancer element, a promoter, and a transcription termination sequence, for example as described in U.S. Pat. No. 5,534,615, which is incorporated in its entirety herein.

Suitable host cells include any prokaryotic (e.g., bacterial), lower eukaryotic (e.g., yeast), or higher eukaryotic (e.g., mammalian) cells. Suitable prokaryotes include eubacteria, such as Gram-negative or Gram-positive organisms, for example, Enterobacteriaceae such as Escherichia (E. coli), Enterobacter, Erwinia, Klebsiella, Proteus, Salmonella (S. typhimurium), Serratia (S. marescans), Shigella, Bacilli (B. subtilis and B. licheniformis), Pseudomonas (P. aeruginosa), and Streptomyces. One useful E. coli cloning host is E. coli 294, although other strains such as E. coli B, E. coli X1776, and E. coli W3110 are suitable.

In addition to prokaryotes, eukaryotic microbes such as filamentous fungi or yeast are also suitable cloning or expression hosts for antibody-encoding vectors. Saccharomyces cerevisiae, or common baker's yeast, is a commonly used lower eukaryotic host microorganism. However, a number of other genera, species, and strains are available and useful, such as Schizosaccharomyces pombe, Kluyveromyces (K. lactis, K fragilis, K bulgaricus K wickeramii, K waltii, K drosophilarum, K thermotolerans, and K. marxianus), Yarrowia, Pichia pastoris, Candida (C. albicans), Trichoderma reesia, Neurospora crassa, Schwanniomyces (S. occidentalis), and filamentous fungi such as, for example Penicillium, Tolypocladium, and Aspergillus (A. nidulans and A. niger).

Useful mammalian host cells include COS-7 cells, HEK293 cells; baby hamster kidney (BHK) cells; Chinese hamster ovary (CHO); mouse sertoli cells; African green monkey kidney cells (VERO-76), and the like.

The host cells used to produce the compositions or antigen binding molecules of this invention may be cultured in a variety of media. Commercially available media such as, for example, Ham's F10, Minimal Essential Medium (MEM), RPMI-1640, and Dulbecco's Modified Eagle's Medium (DMEM) are suitable for culturing the host cells. In addition, any of the media described in Ham et al., Meth. Enz., 1979, 58:44; Barnes et al., Anal. Biochem., 1980, 102:255; and U.S. Pat. Nos. 4,767,704, 4,657,866, 4,927,762, 4,560,655, and 5,122,469, or WO 90/03430 and WO 87/00195 may be used; each of the above-noted references are incorporated by reference herein in their entirety.

Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics, trace elements (defined as inorganic compounds usually present at final concentrations in the micromolar range), and glucose or an equivalent energy source. Any other necessary supplements may also be included at appropriate concentrations that would be known to those skilled in the art.

The culture conditions, such as temperature, pH, and the like, are those previously used with the host cell selected for expression, and will be apparent to the ordinarily skilled artisan.

When using recombinant techniques, the compositions or antigen binding molecules can be produced intracellularly, in the periplasmic space, or directly secreted into the medium. If the antigen binding molecule is produced intracellularly, as a first step, the particulate debris, either host cells or lysed fragments, is removed, for example, by centrifugation or ultrafiltration. For example, Carter et al. (BioTechnology, 1992, 10:163-167, which is incorporated by reference in its entirety herein) describes a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 minutes. Cell debris can be removed by centrifugation.

In some embodiments, the compositions or the antigen binding molecule is produced in a cell-free system. In some embodiments, the cell-free system is an in vitro transcription and translation system as described in Yin et al., mAbs, 2012, 4:217-225, incorporated by reference in its entirety. In some embodiments, the cell-free system utilizes a cell-free extract from a eukaryotic cell or from a prokaryotic cell. In some embodiments, the prokaryotic cell is E. coli. Cell-free expression of the compositions or antigen binding molecules may be useful, for example, where compositions or antigen binding molecules accumulates in a cell as an insoluble aggregate, or where yields from periplasmic expression are low.

Where the compositions or the antigen binding molecule is secreted into the medium, supernatants from such expression systems are generally first concentrated using a commercially available protein concentration filter, for example, an Amicon® or Millipore™ Pelicon® ultrafiltration unit. A protease inhibitor such as PMSF may be included in any of the foregoing steps to inhibit proteolysis and antibiotics may be included to prevent the growth of adventitious contaminants.

The compositions or the antigen binding molecule prepared from the cells can be purified using, for example, hydroxylapatite chromatography, gel electrophoresis, dialysis, and affinity chromatography, with affinity chromatography being a particularly useful purification technique. The suitability of protein A as an affinity ligand depends on the species and isotype of any immunoglobulin Fc domain that is present in the antibody. Protein A can be used to purify antibodies that are based on human 71, y2, or y4 heavy chains (Lindmark et al., J. Immunol. Meth., 1983, 62:1-13). Protein G is useful for all mouse isotypes and for human y3 (Guss et al., EMBO J., 1986, 5:1567-1575).

The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the compositions or antigen binding molecules comprises a CH3 domain, the BakerBond ABX® resin is useful for purification.

Other techniques for protein purification, such as fractionation on an ion-exchange column, ethanol precipitation, Reverse Phase HPLC, chromatography on silica, chromatography on heparin Sepharose®, chromatofocusing, SDS-PAGE, and ammonium sulfate precipitation are also available, and can be applied by one of skill in the art.

Following any preliminary purification step(s), the mixture comprising compositions or antigen binding molecules of interest and contaminants may be subjected to low pH hydrophobic interaction chromatography using an elution buffer at a pH between about 2.5 to about 4.5, generally performed at low salt concentrations (e.g., from about 0 to about 0.25 M salt).

Preparation of the Compositions or Antigen Binding Molecules

Antigen binding molecules may be obtained by any method known to one skilled in the art. Antibodies may be obtained, for example, using the hybridoma method first described by Kohler et al., Nature, 1975, 256:495-497, and/or by recombinant DNA methods {see e.g., U.S. Pat. No. 4,816,567). Monoclonal antibodies may also be obtained, for example, using phage or yeast-based libraries. See e.g., U.S. Pat. Nos. 8,258,082 and 8,691,730.

In the hybridoma method, a mouse or other appropriate host animal is immunized to elicit lymphocytes that produce or are capable of producing antibodies that will specifically bind to the protein used for immunization. Alternatively, lymphocytes may be immunized in vitro. Lymphocytes are then fused with myeloma cells using a suitable fusing agent, such as polyethylene glycol, to form a hybridoma cell. See Goding J. W., Monoclonal Antibodies: Principles and Practice 3^{rd} ed. (1986) Academic Press, San Diego, CA.

The hybridoma cells are seeded and grown in a suitable culture medium that contains one or more substance(s) that inhibit the growth or survival of the unfused, parental myeloma cells. For example, if the parental myeloma cells lack the enzyme hypoxanthine guanine phosphoribosyl transferase (HGP T or HPRT), the culture medium for the hybridomas typically will include hypoxanthine, aminopterin, and thymidine (HAT medium), which substances prevent the growth of HGPRT-deficient cells.

Useful myeloma cells are those that fuse efficiently, support stable high-level production of antibody by the selected antibody-producing cells, and are sensitive media conditions, such as the presence or absence of HAT medium. Among these, preferred myeloma cell lines are murine myeloma lines, such as those derived from MOP-21 and MC-11 mouse tumors (available from the Salk Institute Cell Distribution Center, San Diego, CA), and SP-2 or X63-Ag8-653 cells (available from the American Type Culture Collection, Rockville, MD). Human myeloma and mouse-human heteromyeloma cell lines also have been described for the production of human monoclonal antibodies. See e.g., Kozbor, J. Immunol, 1984, 133:3001.

After the identification of hybridoma cells that produce antibodies of the desired specificity, affinity, and/or biological activity, selected clones may be subcloned by limiting dilution procedures and grown by standard methods. See Goding, supra. Suitable culture media for this purpose include, for example, D-MEM or RPMI-1640 medium. In addition, the hybridoma cells may be grown in vivo as ascites tumors in an animal.

DNA encoding the monoclonal antibodies may be readily isolated and sequenced using conventional procedures (e.g., by using oligonucleotide probes that are capable of binding specifically to genes encoding the heavy and light chains of the monoclonal antibodies). Thus, the hybridoma cells can serve as a useful source of DNA encoding antibodies with the desired properties. Once isolated, the DNA may be placed into expression vectors, which are then transfected into host cells such as bacteria (e.g., E. coli), yeast (e.g., Saccharomyces or Pichia sp.), COS cells, Chinese hamster ovary (CHO) cells, or myeloma cells that do not otherwise produce antibody, to produce the monoclonal antibodies.

Humanized antibodies may be generated by replacing most, or all, of the structural portions of a monoclonal antibody with corresponding human antibody sequences. Consequently, a hybrid molecule is generated in which only the antigen-specific variable, or CDR, is composed of non-human sequence. Methods to obtain humanized antibodies include those described in, for example, Winter and Milstein, Nature, 1991, 349:293-299; Rader et al., Proc. Nat. Acad. Sci. U.S.A., 1998, 95:8910-8915; Steinberger et al., J. Biol. Chem., 2000, 275:36073-36078; Queen et al., Proc. Natl. Acad. Sci. U.S.A., 1989, 86: 10029-10033; and U.S. Pat. Nos. 5,585,089, 5,693,761, 5,693,762, and 6,180,370.

Human antibodies can be generated by a variety of techniques known in the art, for example by using transgenic animals (e.g., humanized mice). See, e.g., Jakobovits et al., Proc. Natl. Acad. Sci. U.S.A., 1993, 90:2551; Jakobovits et al., Nature, 1993, 362:255-258; Bruggermann et al., Year in Immuno., 1993, 7:33; and U.S. Pat. Nos. 5,591,669, 5,589,369 and 5,545,807. Human antibodies can also be derived from phage-display libraries {see e.g., Hoogenboom et al., J. Mol. Biol, 1991, 227:381-388; Marks et al., J. Mol. Biol, 1991, 222:581-597; and U.S. Pat. Nos. 5,565,332 and 5,573,905). Human antibodies may also be generated by in vitro activated B cells {see e.g., U.S. Pat. Nos. 5,567,610 and 5,229,275). Human antibodies may also be derived from yeast-based libraries {see e.g., U.S. Pat. No. 8,691,730).

Disease(s)

In some embodiments, the disease or condition comprises or consists of one or more of multiple sclerosis, Alzheimer's disease, Huntington's disease, Parkinson's disease, epilepsy, brain tumor, stroke, amyotrophic lateral sclerosis, spinal cord and/or brain trauma, a disease or condition which would benefit from enzyme replacement therapy (“ERT”), a neurological disease, chronic inflammatory conditions, acute inflammatory conditions, or bacterial infection. In some embodiments, the disease or condition comprises or consists of autoimmune diseases and infection. In some embodiments, the disease or condition comprises or consists of frontotemporal dementia (“FTD”), viral infections, and/or parasitic infections. In some embodiments, the viral infections comprises or consists of one or more of HIV or West Nile Virus. In some embodiments, parasitic infections comprises or consists of one or more of schistosomiasis and/or trypanosomiasis.

In some embodiments, the disease or condition comprises or consists of multiple sclerosis. Multiple sclerosis (MS) is a prototypical inflammatory demyelinating disease of the central nervous system (CNS). Its clinical manifestations begin typically in the third and fourth decade of life. MS represents a prime cause of neurological disability in young adults and has wide health, psychological, economic and social consequences. MS affects more women than men.

Clinically, MS manifests itself as neurological deficits that frequently exhibit a relapsing and remitting pattern and can resolve completely or can leave residual deficits. The deficits can involve any part of the CNS alone or in combination. Pyramidal-motor, and/or visual manifestations, the latter due either to inflammatory demyelination in the afferent visual pathways (optic neuritis) or in the efferent visual pathways (ocular motility disorders such as internuclear ophthalmoplegia) are among the most common manifestations. Eventually, many people with relapse-onset MS have fewer clinically recognizable relapses and develop a gradual neurological progression.

There are several MS subtypes: relapsing-remitting MS (RRMS), with relapses (flare-ups) of disease separated by periods without clinical progression; secondary progressive, SPMS, which represents the phase of the disease where a gradual neurological deterioration (progression) follows a period of RR disease; and primary progressive, PPMS, where the neurological deterioration is present from the onset, most frequently without superimposed relapses. A rare variant where a few acute exacerbations are superimposed on the gradual PPMS-like course is called progressive-relapsing MS (PRMS). Individuals who have experienced a single typical episode of inflammatory demyelination suggestive of being the first attack of MS but have not had a second event are said to have clinically isolated syndrome (CIS).

There are four key pathological features of MS: (a) inflammation, which is generally believed to be the main trigger of the events leading to CNS tissue damage in the majority of cases; (b) demyelination, the hallmark of MS, where the myelin sheath or the oligodendrocyte cell body is destroyed by the inflammatory process; (c) axonal loss or damage; and (d) gliosis (astrocytic reaction to CNS damage).

The pathological correlate of relapses is inflammation and disruption of the blood-brain barrier, clinical relapses being thought to correspond to fresh waves of inflammatory cell infiltration in the CNS. The pathological correlate of long-term disability and progression is irreversible axonal loss. The acute MS lesion is characterized by inflammatory pass throughs with various immune cells and active demyelination (macrophages with myelin debris in their cytoplasm); when this lesion becomes chronic, there is significant loss of myelin with few if any inflammatory pass throughs and gliosis, which gives lesions their plaque appearance.

In some embodiments, the disease or condition comprises or consists of Alzheimer's disease. Alzheimer's disease is a type of dementia that affects memory, thinking, and behavior. Alzheimer's disease is the most common form of dementia, a general term for memory loss and other cognitive abilities serious enough to interfere with daily life. Alzheimer's disease generally worsens over time. Alzheimer's disease is a multifactorial disorder leading to progressive memory loss and eventually death.

One of the pathological features of the disease is the abnormal accumulation of toxic Aβ peptides in the brain parenchyma. These peptides are cleavage products derived from the amyloid precursor protein (APP) through endoproteolytic cleavage operated by specific secretases, BACE-1 and γ-secretase. APP mutations alter the processing of the protein by shifting the nonamyloidogenic processing towards amyloidogenic processing, which eventually leads to generation of highly fibrillogenic, toxic Aβ1-42 peptides.

Presenilin-1 (PS-1) and presenilin-2 (PS-2) function as a catalytic site for γ-secretase and mutations in PS-1 or PS-2 further increase the production of amyloidogenic Aβ. Human AD neurons also contain intraneuronal inclusions of hyperphosphorylated tau protein, called neurofibrillary tangles. These abnormal protein inclusions alter neuronal function and result in neuron death. Mutations in APP and PS1 have been linked to familial, inherited forms of AD, which account less than 10% of the clinical AD cases. Indeed, the majority of the diagnosed AD patients have a sporadic form of the disease in which the underlying cause remains unknown.

In some embodiments, the disease or condition comprises or consists of Huntington's disease. Huntington's disease (“HD”) is a fatal genetic disorder that causes the progressive breakdown of nerve cells in the brain. It deteriorates a person's physical and mental abilities, usually during their prime working years. Huntington's disease has no cure. HD is known as the quintessential family disease because every child of a parent with HD has a 50/50 chance of inheriting the faulty gene. Today, there are approximately 41,000 symptomatic Americans and more than 200,000 at-risk of inheriting the disease.

The symptoms of HD are described as having ALS, Parkinson's, and Alzheimer's simultaneously. Symptoms usually appear between the ages of 30 to 50 and worsen over a 10 to 25-year period. Ultimately, the weakened individual succumbs to pneumonia, heart failure, or other complications. Symptoms include personality changes and mood swings, forgetfulness and impaired judgment, unsteady gate and involuntary movements, slurred speech and difficulty swallowing and significant weight loss.

Everyone has the gene that causes HD but only those that inherit the expansion of the gene will develop HD and perhaps pass it on to each of their children. Huntington's disease is caused by an expansion of a repeating CAG triplet series in the huntingtin gene on chromosome 4, which results in a protein with an abnormally long polyglutamine sequence. HD is one of a larger family of polyglutamine repeat disorders, all of which are neurodegenerative diseases. In some embodiments, the disease or condition comprises or consists of a polyglutamine repeat disorders.

The normal function of huntingtin is not known, but the expanded polyglutamine sequence in the huntingtin protein is in some way toxic to brain cells. Just as in other polyglutamine expansion disorders, certain neurons appear to be more vulnerable to damage in HD. Atrophy is most marked in the corpus striatum of the basal ganglia, including the caudate and putamen. In later phases of the disease, other regions of the brain are also affected.

Every person who inherits the expanded HD gene will eventually develop the disease. It is inherited in an autosomal dominant fashion, so that each child of an affected parent has a 50% chance of developing the disease. There is currently no cure or treatment which can halt, slow, or reverse the progression of the disease.

In some embodiments, the disease or condition comprises or consists of Parkinson's disease or a Lewy body dementia disease. Parkinson's disease is a brain disorder that leads to shaking, stiffness, slowness of movement, and difficulty with walking, balance, and coordination. Other symptoms may include depression, difficulty swallowing, chewing, and speaking, urinary problems or constipation, skin problems, and sleep disruption. Parkinson's symptoms usually begin gradually and get worse over time. As the disease progresses, people may have difficulty walking and talking. Both men and women can have Parkinson's disease. However, the disease affects about 50 percent more men than women.

Parkinson's disease occurs when nerve cells, or neurons, in an area of the brain that controls movement become impaired and/or die. Normally, these neurons produce dopamine. When the neurons die or become impaired, they produce less dopamine, which causes the movement problems of Parkinson's. It is not clear what causes neurons that produce dopamine to die.

People with Parkinson's also lose the nerve endings that produce norepinephrine, the main chemical messenger of the sympathetic nervous system, which controls many automatic functions of the body, such as heart rate and blood pressure. The loss of norepinephrine might help explain some of the non-movement features of Parkinson's, such as fatigue, irregular blood pressure, decreased movement of food through the digestive tract, and sudden drop in blood pressure when a person stands up from a sitting or lying-down position.

Many brain cells of people with Parkinson's contain Lewy bodies, unusual clumps of the protein alpha-synuclein. There is an effort underway to better understand the normal and abnormal functions of alpha-synuclein and its relationship to genetic mutations that impact Parkinson's disease and Lewy body dementia.

Although some cases of Parkinson's appear to be hereditary, and a few can be traced to specific genetic mutations, in most cases the disease occurs randomly and does not seem to run in families. Many researchers now believe that Parkinson's disease results from a combination of genetic factors and environmental factors such as exposure to toxins.

One clear risk factor for Parkinson's is age. Although most people with Parkinson's first develop the disease at about age 60, about 5 to 10 percent of people with Parkinson's have “early-onset” disease, which begins before the age of 50. Early-onset forms of Parkinson's are often, but not always, inherited, and some forms have been linked to specific gene mutations.

There is no cure for Parkinson's disease. Treatments include drugs that increase the level of dopamine in the brain and drugs that affect other chemicals in the body. And drugs that help control non-motor functions. The main therapy for Parkin's disease is levodopa, also called L-dopa. Usually, people take levodopa along with another medication called carbidopa.

In some embodiments, the disease or condition comprises or consists of epilepsy. Epilepsy is a group of neurological diseases characterized by recurrent epileptic seizures. Epileptic seizures are episodes that can vary from brief and nearly undetectable periods to long periods of vigorous shaking. These episodes can result in physical injuries, including broken bones.

In epilepsy, seizures have a tendency to recur and, as a rule, have no immediate underlying cause. Isolated seizures that are provoked by a specific cause such as poisoning are not deemed to represent epilepsy. People with epilepsy may be treated differently in various areas of the world and experience varying degrees of social stigma due to their condition.

The underlying mechanism of epileptic seizures is excessive and abnormal neuronal activity in the cortex of the brain. Most of the time the reason is unknown. Some cases occur as the result of brain injury, stroke, brain tumors, infections of the brain, or birth defects. Known genetic mutations are directly linked to only a small proportion of cases.

Diagnosis often involves ruling out other conditions that might cause similar symptoms, such as fainting, and determining if another cause of seizures is present, such as alcohol withdrawal or electrolyte problems. This may be partly done by imaging and blood tests. Epilepsy can often be confirmed with an electroencephalogram (EEG), but a normal test does not rule out the condition.

Epilepsy that occurs as a result of other issues may be preventable. Seizures are controllable with medication in about 70% of cases; inexpensive anti-seizure medications are often available. In those whose seizures do not respond to medication, surgery, neurostimulation, or dietary changes may be considered. Not all cases of epilepsy are lifelong and many people improve to the point that treatment is no longer needed.

In some embodiments, the disease or condition comprises or consists of brain tumor. A brain tumor is a mass or growth of abnormal cells in the brain. A brain tumor can either be cancerous (malignant) or benign. Cancerous tumors can be divided into primary tumors, which start within the brain, and secondary tumors, which are metastatic tumors.

All types of brain tumors may produce symptoms that vary depending on the part of the brain involved. Symptoms may include headaches, seizures, problems with vision, and vomiting and mental changes. Other symptoms may include difficulty walking, speaking, or with sensations.

The cause of most brain tumors is unknown. Uncommon risk factors include exposure to vinyl chloride, Epstein-Barr virus, ionizing radiation, and inherited syndromes. Diagnosis is usually by medical examination with a CT or MRI. Results are often confirmed by a biopsy.

Many different types of brain tumors exist including, without limitation, gliomas, meningiomas, acoustic neuromas, pituitary adenomas, medulloblastomas, germ cell tumors, atrocytomas, and craniopharyngioma. In some embodiments, the brain tumor comprises or consists of a glioma. A glioma begins in the brain or spinal cord and include astrocytomas, ependymomas, glioblastomas, oligoastrocytomas and oligodendrogliomas.

In some embodiments, the brain tumor comprises or consists of a meningioma. A meningioma is a tumor that arises from the membranes that surround your brain and spinal cord (meninges). Most meningiomas are noncancerous.

In some embodiments, the brain tumor comprises or consists of an acoustic neuroma. An acoustic neuroma is normally a benign tumor that develops on the nerves that control balance and hearing leading from your inner ear to your brain.

In some embodiments, the brain tumor comprises or consists of a pituitary adenoma. Pituitary adenomas are mostly benign tumors that develop in the pituitary gland at the base of the brain. Pituitary adenomas can affect the pituitary hormones with effects throughout the body.

In some embodiments, the brain tumor comprises or consists of a medulloblastoma. Meduloblastomas are the most common cancerous brain tumors in children. A medulloblastoma starts in the lower back part of the brain and tends to spread through the spinal fluid. A medulloblastoma tumor is less common in adults, but they do occur.

In some embodiments, the brain tumor comprises or consists of a germ cell tumor. Germ cell tumors may develop during childhood where the testicles or ovaries will form. But sometimes germ cell tumors affect other parts of the body, such as the brain.

In some embodiments, the brain tumor comprises or consists of an astrocytoma. An astrocytoma begins in cells called astrocytes that support nerve cells. Astrocytoma can be a slow-growing tumor, or it can be an aggressive cancer that grows quickly.

In some embodiments, the brain tumor comprises or consists of a craniopharyngioma. A craniopharyngioma is a rare, noncancerous tumor that starts near the brain's pituitary gland, which secretes hormones that control many body functions. As the craniopharyngioma slowly grows, it can affect the pituitary gland and other structures near the brain.

In some embodiments, the brain tumor comprises or consists of a secondary, or metastatic, brain tumor. A secondary or metastatic brain tumor results from cancer that starts elsewhere in the body and then spreads (metastasizes) to the brain. Secondary, or metastatic, brain tumors are about four times as common as primary brain tumors with about half of metastases coming from lung cancer. Primary brain tumors occur in around 250,000 people a year globally, making up less than 2% of cancers. In children younger than 15, brain tumors are second only to acute lymphoblastic leukemia as the most common form of cancer.

In some embodiments, the disease or condition comprises or consists of stroke. Stroke is a multiphasic process in which initial cerebral ischemia is followed by secondary injury from immune responses to ischemic brain components. Stroke is the fourth leading cause of death and the leading cause of disability.

Thrombolytic therapy and endovascular removal of thrombi are the only approved therapies for acute ischemic stroke and must be instituted early after the ischemic event. Because of this limited window of therapeutic time, only a small percentage of stroke patients have so far benefited from these interventions.

On the other hand, innate and adaptive immune responses initiated after cerebral ischemia unfold over days to weeks after stroke. Cerebral ischemia causes release of highly immunogenic cellular components, or danger/damage-associated molecular patterns (DAMPs), from the brain into the systemic circulation. These DAMPs activate and recruit peripheral innate and adaptive immune cells to ischemic brain regions. Experimental manipulations suggest that both toxic and protective inflammatory processes are activated after stroke, with toxic effects including generation of proinflammatory cytokines, proteases and reactive oxygen species by inflammatory cells, and protective effects consisting of clearance of injured tissue by myeloid cells and the establishment of a regenerative environment.

Early post-stroke innate immune responses are be amplified by TREM1. In a rodent model of transient focal cerebral ischemia, TREM1 is selectively induced in peripheral myeloid cells that traffic to the ischemic brain.

Inhibition of TREM1 reduces stroke injury. Peripheral TREM1 induction occurs not only in spleen, but also in intestinal inflammatory macrophage subsets following sympathetic-mediated increases in gut permeability. TREM1 amplifies gut permeability, the peripheral innate immune response, and bacterial translocation to the periphery. The peripheral myeloid cells worsen cerebral injury via TREM1 amplification of immune responses to both sterile brain components and gut microbial pathogens.

In some embodiments, the disease or condition comprises or consists of amyotrophic lateral sclerosis. Amyotrophic lateral sclerosis, also known as motor neuron disease (“MND”), ALS, or Lou Gerhig's disease, is a disease that causes the death of neurons controlling involuntary muscles. Amyotrophic lateral sclerosis is a progressive nervous system disease that affects nerve cells in the brain and spinal cord, causing loss of muscle control.

Amyotrophic lateral sclerosis is characterized by stiff muscles, muscle twitching, and gradually worsening weakness due to muscles decreasing in size. It can begin with a weakness in the arms or with difficulty swallowing. Some people develop mild difficulties with thinking and behavior. Most people have pain. Most lose the ability to walk, use their hands, speak, swallow, and breathe.

Most of the time the cause of ALS is not known. ALS causes the motor neurons to gradually deteriorate and then die. Motor neurons extend from the brain to the spinal cord to muscles throughout the body. When motor neurons are damaged, they stop sending messages to the muscles, so the muscles can't function.

In some embodiments, the disease or condition comprises spinal cord and/or brain trauma. Brain or spinal injuries differ in complexity and severity and range in effect from mild to severe. In the United States, there are an estimated 2.87 million Traumatic Brain Injury (TBI)-related emergency department visits, hospitalizations, and deaths each year, while there are nearly 18,000 new spinal cord injuries each year.

In some embodiments, the disease or condition comprises or consists of TBI or chronic traumatic encephalopathy. Symptoms of TBI can range from brief loss of consciousness, headache, and confusion to those symptoms plus persistent headache, repeated vomiting or nausea, and convulsions or seizures. Chronic traumatic encephalopathy, a progressive disease caused by repeated brain trauma, can lead to memory loss, confusion, personality and behavior changes, and difficulty with attention, organizing thoughts, and balance and motor skills. Spinal cord injuries often impair body function, ranging from limited or weak movement to no function below the level of the injury. There currently are no cures.

In some embodiments, the disease or condition comprises or consists of a disease or condition which would benefit from enzyme replacement therapy (“ERT”). Enzyme replacement therapy (“ERT”) is a medical treatment which replaces an enzyme that is deficient or absent in the body. Usually, this is done by giving the patient an intravenous (IV) infusion of a solution containing the enzyme.

ERT is currently available for some lysosomal storage diseases such as Gaucher disease, Fabry disease, MPSI, MPSII (Hunter syndrome), MPS VI, and Pompe disease. ERT does not correct the underlying genetic defect, but it increases the concentration of the enzyme that the patient is lacking. ERT has also been used to treat patients with severe combined immunodeficiency (SCID) resulting from an adenosine deaminase deficiency (ADA-SCID).

In some embodiments, the disease or condition comprises or consists of frontotemporal dementia (“FTD”). Frontotemporal dementia is an umbrella term for a group of uncommon brain disorders that primarily affect the frontal and temporal lobes of the brain. These areas of the brain are generally associated with personality, behavior and language.

In frontotemporal dementia, portions of these lobes shrink (atrophy). Signs and symptoms vary, depending on which part of the brain is affected. Some people with frontotemporal dementia have dramatic changes in their personality and become socially inappropriate, impulsive, or emotionally indifferent, while others lose the ability to use language properly.

Frontotemporal dementia is often misdiagnosed as a psychiatric problem or as Alzheimer's disease. But frontotemporal dementia tends to occur at a younger age than does Alzheimer's disease. Frontotemporal dementia often begins between the ages of 40 and 65.

Signs and symptoms of frontotemporal dementia can be different from one individual to the next. Signs and symptoms get progressively worse over time, usually over years. Clusters of symptom types tend to occur together, and people may have more than one cluster of symptom types.

The most common signs of frontotemporal dementia involve extreme changes in behavior and personality, such as increasing inappropriate social behavior, loss of empathy and other interpersonal skills, such as having sensitivity to another's feelings, lack of judgment, loss of inhibition, lack of interest (apathy), which can be mistaken for depression, repetitive compulsive behavior, such as tapping, clapping or smacking lips, a decline in personal hygiene, changes in eating habits, usually overeating or developing a preference for sweets and carbohydrates, eating inedible objects, and impulsively wanting to put things in the mouth.

Some subtypes of frontotemporal dementia lead to language problems or impairment or loss of speech. Primary progressive aphasia, semantic dementia, and progressive agrammatic (confluent) aphasia are all considered to be frontotemporal dementia. Problems in this category include increasing difficulty in using and understanding written and spoken language, such as having trouble finding the right word to use in speech or naming objects, trouble naming things, possibly replacing a specific word with a more general word such as “it” for pen, no longer knowing word meanings, having hesitant speech that may sound telegraphic, and making mistakes in sentence construction.

It is not always clear what causes frontal temporal dementia. In frontotemporal dementia, the frontal and temporal lobes of the brain shrink. In addition, certain substances accumulate in the brain. There are genetic mutations that have been linked to frontotemporal dementia. But more than half of the people who develop frontotemporal dementia have no family history of dementia. Recently, researchers have confirmed shared genetics and molecular pathways between frontotemporal dementia and amyotrophic lateral sclerosis (ALS).

In some embodiments, the disease or condition comprises or consists of viral infections. In some embodiments, the viral infections comprise or consist of HIV or West Nile Virus. The human immunodeficiency viruses (HIV) are two species of Lentivirus (a retrovirus) that infect humans. Over time, HIV causes acquired immunodeficiency syndrome or AIDS. AIDS I is a condition in which progressive failure of the immune system allows life-threatening opportunistic infections and cancer to thrive. Without treatment, average survival time after infection with HIV is estimated to be 9 to 11 years.

In most cases, HIV is a sexually transmitted infection and occurs by contact with or transfer of a blood, pre-ejaculate, semen, or vaginal fluids. Non-sexual transmission can occur from an infected mother to her infant during pregnancy, during childbirth by exposure to her blood or vaginal fluid, and through breast milk. Within these bodily fluids, HIV is present as both free virus particles and virus within infected immune cells.

HIV infects vital cells in the human immune system, such as helper T cells (specifically CD4⁺ T cells), macrophages, and dendritic cells. HIV infection leads to low levels of CD4⁺ T cells through a number of mechanisms, including abortively infected T cells, apoptosis of uninfected bystander cells, direct viral killing of infected cells, and killing of infected CD4⁺ T cells by CD8⁺ cytotoxic T lymphocytes that recognize infected cells. When CD4⁺ T cell numbers decline below a critical level, cell-mediated immunity is lost, and the body becomes progressively more susceptible to opportunistic infections, leading to the development of AIDS.

West Nile virus (WNV) is the leading cause of mosquito-borne disease in the continental United States. It is most commonly spread to people by the bite of an infected mosquito. Cases of West Nile virus occur during mosquito season, which starts in the summer and continues through fall. There are no vaccines to prevent or medications to treat WNV in people.

Fortunately, most people infected with WNV do not feel sick. About 1 in 5 people who are infected develop a fever and other symptoms. About 1 out of 150 infected people develop a serious, sometimes fatal, illness. One can reduce your risk of WNV by using insect repellent and wearing long-sleeved shirts and long pants to prevent mosquito bites.

About 1 in 150 people who are infected develop a severe illness affecting the central nervous system such as encephalitis (inflammation of the brain) or meningitis (inflammation of the membranes that surround the brain and spinal cord). Symptoms of severe illness include high fever, headache, neck stiffness, stupor, disorientation, coma, tremors, convulsions, muscle weakness, vision loss, numbness and paralysis. Severe illness can occur in people of any age; however, people over 60 years of age are at greater risk.

People with certain medical conditions, such as cancer, diabetes, hypertension, kidney disease, and people who have received organ transplants, are also at greater risk. Recovery from severe illness might take several weeks or months. Some effects to the central nervous system might be permanent. About 1 out of 10 people who develop severe illness affecting the central nervous system die.

No vaccine or specific antiviral treatments for West Nile virus infection are available. Over-the-counter pain relievers can be used to reduce fever and relieve some symptoms. In severe cases, patients often need to be hospitalized to receive supportive treatment, such as intravenous fluids, pain medication, and nursing care.

In some embodiments, the disease or condition comprises or consists of a parasitic infection. In some embodiments, the parasitic infection comprises of consists of schistosomiasis. In some embodiments, the parasitic infection comprises or consists of trypanosomiasis.

Schistosomiasis, also known as bilharzia, is a disease caused by infection with freshwater parasitic worms in certain tropical and subtropical countries. The parasite can be found in sub-Saharan Africa, the Middle East, Southeast Asia, and the Caribbean. Freshwater becomes contaminated from infected animal or human urine or feces. The parasites penetrate human skin to enter the bloodstream and migrate to the liver, intestines, and other organs. A rash, itchy skin, fever, chills, cough, headache, belly pain, joint pain, and muscle aches are symptoms.

Although the worms that cause schistosomiasis are not found in the United States, people are infected worldwide. In terms of impact this disease is second only to malaria as the most devastating parasitic disease. Schistosomiasis is considered one of the neglected tropical diseases. The parasites that cause schistosomiasis live in certain types of freshwater snails. The infectious form of the parasite, known as cercariae, emerge from the snail into the water. One can become infected when skin comes in contact with contaminated freshwater.

Schistosomiasis is caused by some species of blood trematodes (flukes) in the genus Schistosoma. The three main species infecting humans are Schistosoma haematobium, S. japonicum, and S. mansoni. Three other species, more localized geographically, are S. mekongi, S. intercalatum, and S. guineensis (previously considered synonymous with S. intercalatum). There have also been a few reports of hybrid schistosomes of cattle origin (S. haematobium, ×S. bovis, ×S. curassoni, ×S. mattheei) infecting humans. Unlike other trematodes, which are hermaphroditic, Schistosoma spp. are dioecous (individuals of separate sexes). In addition, other species of schistosomes, which parasitize birds and mammals, can cause cercarial dermatitis in humans but this is clinically distinct from schistosomiasis.

A rash or itchy skin may develop days after being infected. Fever, chills, cough, and muscle aches can begin within 1-2 months of infection. Most people have no symptoms at this early phase of infection.

When adult worms are present, the eggs that are produced usually travel to the intestine, liver, or bladder, causing inflammation or scarring. Children who are repeatedly infected can develop anemia, malnutrition, and learning difficulties. After years of infection, the parasite can also damage the liver, intestine, lungs, and bladder. Eggs are even sometimes found in the brain or spinal cord and can cause seizures, paralysis, or spinal cord inflammation. Symptoms of schistosomiasis are caused by the body's reaction to the eggs produced by worms, not by the worms themselves.

Safe and effective medication is available for treatment of both urinary and intestinal schistosomiasis. Praziquantel, a prescription medication, is taken for 1-2 days to treat infections caused by all schistosome species.

Trypanosomiasis is the name of several diseases in caused parasitic protozoan trypanosomes of the genus Trypanosoma. In humans this includes African trypanosomiasis and Chaga's disease.

In some embodiments, the disease or conditions comprises or consists of African trypanosomiasis. African trypanosomiasis, or African sleeping sickness, is caused by either Tryponosoma brucei gambiense or Trypanosoma brucei rhodesiense. It threatens some 65 million people in sub-Saharan Africa, especially in rural areas and populations disrupted by war or poverty.

The disease is characterized by two stages. Initially, the first stage of the disease is characterized by fevers, headaches, itchiness, and joint pains, beginning one to three weeks after the bite. Weeks to months later, the second stage begins with confusion, poor coordination, numbness, and trouble sleeping. Diagnosis is by finding the parasite in a blood smear or in the fluid of a lymph node. A lumbar puncture is often needed to tell the difference between first and second stage disease.

The second phase of the disease, the neurological phase (also called the meningoencephalic stage), begins when the parasite invades the central nervous system by passing through the blood brain barrier. Progression to the neurological phase occurs after an estimated 21-60 days in case of T. b. rhodesiense infection and 300-500 days in case of T. b. gambiense infection. Sleep-wake disturbances are a leading feature of neurological stage and give the disease its common name African sleeping sickness. Infected individuals experience a disorganized and fragmented sleep-wake cycle. Those affected experience sleep inversion resulting in daytime sleep and somnolence, and nighttime periods of wakefulness and insomnia. Additionally, those affected also experience episodes of sudden sleepiness.

Neurological symptoms include tremor, general muscle weakness, hemiparesis, paralysis of a limb, abnormal muscle tone, gait disturbance, ataxia, speech disturbances, paraesthesia, hyperaesthesia, anaesthesia, visual disturbance, abnormal reflexes, seizures, and coma. Parkinson-like movements might arise due to non-specific movement disorders and speech disorders. Individuals may exhibit psychiatric symptoms which may sometimes dominate the clinical diagnosis and may include aggressiveness, apathy, irritability, psychotic reactions, hallucinations, anxiety, emotional lability, confusion, mania, attention deficit, and delirium.

Without treatment, the disease is invariably fatal, with progressive mental deterioration leading to coma, systemic organ failure, and death. An untreated infection with T. b. rhodesience will cause death within months whereas an untreated infection with T. b. gambiense will cause death after several years. Damage caused in the neurological phase is irreversible

Treatment of the second stage may involve eflornithine or a combination of nifurtimox and eflomithine for TbG. Fexinidazole is a more recent treatment that can be taken by mouth, for either stages of TbG. While melarsoprol works for both types, it is typically only used for TbR, due to serious side effects. Without treatment sleeping sickness typically results in death.

Diagnosis

The methods and compositions of the subject invention for delivering one or more substance(s) to the brain in a subject in need thereof comprises or consists of an antigen binding molecule that binds an antigen on peripheral immune cells as set forth herein. In some embodiments, the subject is in need of diagnosis. Therapeutic selection and monitoring are hampered by the lack of sensitive central nervous system immune biomarkers. As such, existing imaging strategies cannot distinguish between toxic and beneficial immune responses. For example, there is a critical need for non-invasive molecular imaging to accurately track and quantify the movement of cells, such as myeloid cells, to the brain. In MS patients, for example, there is a need to discriminate between active disease and remission.

In some embodiments, the subject is in need of diagnosis. In some embodiments, the subject is in need for diagnosis is for one or more of disease(s) or condition(s), such as those set forth in this application. In some embodiments, the subject is in need of diagnosis for one or more disease(s) or condition(s) comprising or consisting of one or more of multiple sclerosis, Alzheimer's disease, Huntington's disease, Parkinson's disease, epilepsy, brain tumor, stroke, amyotrophic lateral sclerosis, spinal cord and/or brain trauma, a disease or condition which would benefit from enzyme replacement therapy (“ERT”), a neurological disease, chronic inflammatory conditions, acute inflammatory conditions, or bacterial infection. In some embodiments, the disease or condition comprises or consists of autoimmune diseases and infection. In some embodiments, the disease or condition comprises or consists of frontotemporal dementia (“FTD”), viral infections, and/or parasitic infections. In some embodiments, the viral infections comprises or consists of one or more of HIV or West Nile Virus. In some embodiments, parasitic infections comprises or consists of one or more of schistosomiasis and/or trypanosomiasis.

In some embodiments, the disease of condition comprises or consists of one or more of the stage(s) of multiple sclerosis. In some embodiments, the one or more stage(s) of multiple sclerosis comprise or consist of one or more of active disease and remission. In some embodiments, diagnosis comprises or consists of distinguishing between one or more stage(s) of disease.

In some embodiments, the one or more substance(s) comprises or consist of one or more diagnostic or theranostic substance(s). In some embodiments, the one or more diagnostic or theranostic substance(s) comprises or consists of one or more MRI and/or PET probe(s), radiolabel(s), or isotope(s).

Positron emission tomography (PET) is a highly sensitive and noninvasive nuclear imaging technology widely used for preclinical and clinical imaging of diseases. A PET imaging agent, in its most basic form, is thus comprised of a targeting entity (to visualize the biomarker of interest) and a positron-emitting radionuclide.

To this end, a number of new tracers, often based on antibody platforms, have emerged for application in immunotherapy settings. By exploiting the high specificity of antibodies for their targets, as well as the high sensitivity of PET, these PET agents have demonstrated immense potential for the visualization of immune targets and in some cases may be used to stratify potential responders.

Radiometals as radiolabels are becoming increasingly accessible and are utilized frequently in the design of radiotracers for imaging. Nuclear properties ranging from the emission of γ-ray and β⁺-particles (imaging) to Auger electron, β⁻ and α-particles (therapy) in combination with long half-lives are ideally matched with the relatively long biological half-life of monoclonal antibodies in vivo.

Radiometal labeling of antibodies requires the conjugation of a metal chelate to the antibody (See, for example, Boros et al., Chemical aspects of metal ion chelation in the synthesis and application antibody-based radiotracers, J. Labelled Comp Radiopharm 2018 July; 61(9): 652-671, which is incorporated by reference herein in its entirety). This chelate must coordinate the metal under mild conditions required for the handling of antibodies, as well as provide high kinetic, thermodynamic, and metabolic stability once the metal ion is coordinated in order to prevent in vivo release of the radiolabel before the target site is reached.

Common radiolabels are set forth in Table 2.

TABLE 2
Common radionuclides used in PET imaging and their relevant
properties

| | | | Decay mode | |
|---|---|---|---|---|
| Radio | Half-life | | (% branching | Production |
| nuclide | t_{1/2} | | ratio) | route(s) |
| ⁶⁴Cu | 12.701 | h | ϵ+β+ (61.5%); | ⁶⁴Ni(p, n); ⁶⁴Cu |
| | | | β+ (17.60%); | |
| | | | β− (38.5%) | |
| ⁶⁷Cu | 61.83 | h | β− (100%) | ⁶⁸Zn(p, 2p); ⁶⁷Cu; |
| | | | | ⁷⁰Zn(p, α); ⁶⁷Cu; |
| | | | | ⁶⁷Zn(n, p); ⁶⁷Cu; |
| | | | | ⁶⁸Zn(γ, p); ⁶⁷Cu |
| ⁶⁷Ga | 3.2617 | d | ϵ (100%) | ^{nat}Zn(p, x); ⁶⁷Ga; |
| | | | | ⁶⁸Zn(p, 2n); ⁶⁷Ga |
| ⁸⁶Y | 14.74 | h | ϵ+β+ (100%); | ⁸⁶Sr(p, n); ⁸⁶Y |
| | | | β+ (31.9%) | |
| ⁸⁹Zr | 78.41 | h | ϵ+β+ (100%); | ⁸⁹Y(p, n); ⁸⁹Zr |
| | | | β+ (22.74%) | |
| ⁹⁰Y | 64.00 | h | β− (100%) | ⁹⁰Sr/⁹⁰Y |
| ^{99m}Tc | 6.01 | h | β− (0.0037%); | ⁹⁹Mo/^{99m}Tc |
| | | | IT (99.9963%) | |
| ¹¹¹In | 2.8047 | d | ϵ (100%) | ¹¹¹Cd(p, n); ^{111m,g}In; |
| | | | | ¹¹²Cd(p, 2n); ^{111m,g}In |
| ¹²³I | 13.2234 | h | ϵ (100%) | ¹²⁴Xe(p, 2n); |
| | | | | ¹²³Cs/¹²³Xe/¹²³I; |
| | | | | ¹²⁴Xe (p, pn); |
| | | | | ¹²³I; ¹²³Te(p, n); ¹²³I |
| ¹²⁴I | 4.1760 | d | ϵ+β+ (100%); | ¹²⁴Te(p, n); ¹²⁴I |
| | | | β+ (22.7%) | |
| ¹³¹I | 8.0252 | d | β− (100%) | ¹³⁰Te(n, γ); ¹³¹Te/¹³¹I |
| ¹⁷⁷Lu | 6.647 | d | β− (100%) | ¹⁷⁶Lu(n, γ); ¹⁷⁷Lu; |
| | | | | ¹⁷⁶Yb(n, γ); ¹⁷⁷Yb/¹⁷⁷Lu |
| ²¹³Bi | 45.59 | m | α(2.20%); | ²²⁵AC/²¹³Bi |
| | | | β− (97.80%) | |
| ²²⁵Ac | 10.0 | d | α(100%) | ²²⁹Th/²²⁵Ra/²²⁵Ac |

| | GS-GS | | |
|---|---|---|---|
| Radio | Q-value | Particle end-point | |
| nuclide | (keV) | energy/keV | Application |
| ⁶⁴Cu | 1675.03 (⁶⁴Ni) | β+ 653.03; | Immuno-PET and RIT |
| | 579.4 (⁶⁴Zn) | β− 579.4 | |
| ⁶⁷Cu | 561.7 | β− 561.7 | RIT (Immuno-SPECT) |
| ⁶⁷Ga | 1000.8 | Auger and CE | Immuno-SPECT and |
| | | | RIT |
| ⁸⁶Y | 5240 | β+ 3141 | Immuno-PET |
| ⁸⁹Zr | 2833 | β+ 902 | Immuno-PET |
| ⁹⁰Y | 2280.1 | β− 2280.1 | RIT |
| ^{99m}TC | Excited state | β− 435.9 | Immuno-SPECT |
| | (parent) level: | | |
| | 142.68 | | |
| ¹¹¹In | 862 | Auger and CE | Immuno-SPECT and |
| | | | RIT |
| ¹²³I | 1228.6 | Auger and CE | RIT (Immuno-SPECT) |
| ¹²⁴I | 3159.6 | β+ 2137.6 | Immuno-PET |
| ¹³¹I | 970.8 | β− 806.9; | RIT |
| | | Auger and CE | |
| ¹⁷⁷Lu | 498.3 | β− 498.3; | RIT (Immuno-SPECT) |
| | | Auger and CE | |
| ²¹³Bi | β− 1423; | β− 1423; α 5875; | RIT |
| | α 5988 | Auger and CE | |
| ²²⁵Ac | 5935.1 | α 5830; | RIT |
| | | Auger and CE | |

Conjugating of chelator and subsequent radiolabeling of mAbs requires chemical reactions on the protein. Functionalization usually involves post-translational modification of amino-acid side chains (particularly peptide bond formation using the primary amine group of lysine residues), derivatization of cysteine sulfhydryl groups, or site-specific labelling of glycans using chemical and/or enzymatic methods. More recent protein engineering routes have also exploited site-specific enzymatic ligation with prominent methods including transglutaminase derivatization, sortase coupling and formylglycine reactions to produce a range of ADCs. While the chemical nature of the linker group plays an important role in determining the metabolic stability and pharmacokinetics of a radiolabeled mAb, an equally important decision revolves around the choice of the radiometal ion and the chelation chemistry used to produce thermodynamically and kinetically stable radiometal ion complexes.

Table 3 provides common chemical structure of chelates.

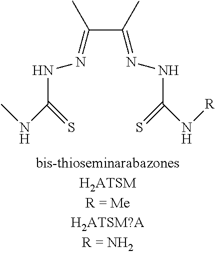

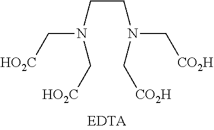 [SMILES: O=C(O)CN(CCN(CC(=O)O)CC(=O)O)CC(=O)O]

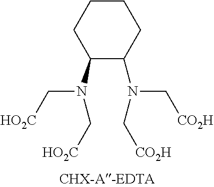 [SMILES: O=C(O)CN(CC(=O)O)C1CCCC[C@@H]1N(CC(=O)O)CC(=O)O]

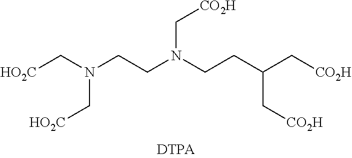 [SMILES: O=C(O)CC(CCN(CCN(CC(=O)O)CC(=O)O)CC(=O)O)CC(=O)O]

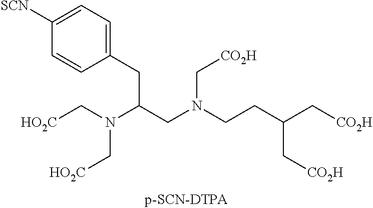

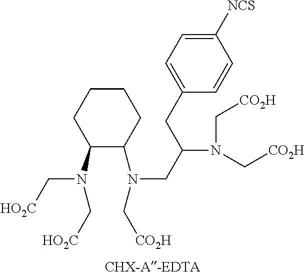

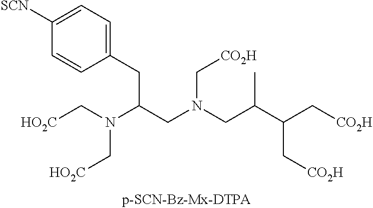

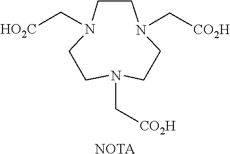 [SMILES: O=C(O)CN1CCN(CC(=O)O)CCN(CC(=O)O)CC1 ; "NOTA"]

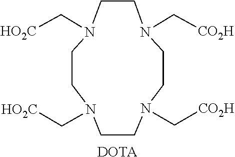

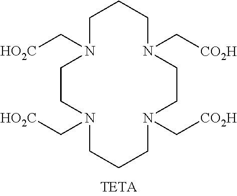 [SMILES: O=C(O)CN1CCCN(CC(=O)O)CCN(CC(=O)O)CCCN(CC(=O)O)CC1]

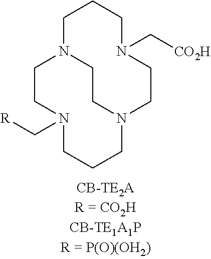

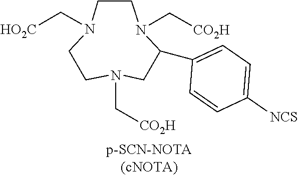

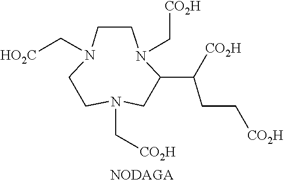

As antibodies have long biological half-lives in vivo, they are well suited for labelling with the long-lived radiometals. After a chelator is attached to the antibody platform of choice, the compound may be incubated with a radiometal, purified, and administered to the patient in need. Any radiometal may be used as set forth herein and as known to one skilled in the art including, for example, without limitation, ⁶⁴Cu or ⁸⁹Zr.

PET scans can then be performed. In preclinical studies, these imaging sessions are often spread out over a long period of time, depending on the radionuclide and targeting platform in use. By imaging several times after injection of the tracer, the optimal imaging timepoint may be determined, at which the signal-to-background ratio is ideal. In clinical scenarios, this is often not feasible and a single imaging session is often performed.

In immunotherapy settings, PET studies have unique considerations relative to other imaging studies which may target, for example, overexpressed molecules on cancer cell surfaces. However, the real potential of PET in these settings is to noninvasively monitor the infiltration, distribution and activation of peripheral immune cells in the brain over time and in response to various therapeutic interventions. In this way, these molecular imaging techniques are unrivalled and hold the capability to revolutionize the treatment paradigm.

MRI can also be used in conjunction with PET (i.e., using a PET/MR scanner). MRI scanners use strong magnetic fields, magnetic field gradients, and radio waves to generate images of the organs in the body. MRI does not involve X-rays or the use of ionizing radiation, which distinguishes it from CT and PET scans.

Certain atomic nuclei are able to absorb radio frequency energy when placed in an external magnetic field. The resultant evolving spin polarization can induce an RF signal in a radio frequency coil and thereby be detected. In clinical and research MRI, hydrogen atoms are most often used to generate a macroscopic polarization that is detected by antennas close to the subject being examined. Hydrogen atoms are naturally abundant in humans and other biological organisms, particularly in water and fat. For this reason, most MRI scans essentially map the location of water and fat in the body, and provide an anatomical reference frame for the PET scan.

Pulses of radio waves excite the nuclear spin energy transition, and magnetic field gradients localize the polarization in space. By varying the parameters of the pulse sequence, different contrasts may be generated between tissues based on the relaxation properties of the hydrogen atoms therein.

Dosage

Forms of the compositions or antigen binding molecules are provided in some embodiments. Dosage forms can be administered to subjects by various routes including, but not limited to, IV, intramuscular, subcutaneous, intraperitoneal, intravitreal, or intrathecal administration.

Suitable vehicles that can be used to provide parenteral dosage forms are well known to those skilled in the art. Examples include, but are not limited to, water for injection; aqueous vehicles such as for example including, but not limited to, sodium chloride injection, ringer's injection, dextrose injection, dextrose and sodium chloride injection, lactated ringer's injection; water miscible vehicles such as for example including, but not limited to, ethyl alcohol, polyethylene glycol, and polypropylene glycol; and non-aqueous vehicles, such as, for example including, but not limited to, corn oil, cottonseed oil, peanut oil, sesame oil, ethyl oleate, isopropyl myristate, and benzyl benzoate.

A doctor will determine the dosage which she considers most appropriate according to the age, weight, condition, and other factors specific to the subject to be treated.

In certain embodiments, exemplary doses of compositions or antigen binding molecules include milligram or microgram amounts of the antibody per kilogram of subject or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram). In certain embodiment, the dosage of the compositions or antigen binding molecules provided herein, based on weight, administered to prevent, treat, manage, or ameliorate a disorder, or one or more symptom(s) thereof in a subject is 0.1 mg/kg, 1 mg/kg, 2 mg/kg, 3 mg/kg, 4 mg/kg, 5 mg/kg, 6 mg/kg, 10 mg/kg, or 15 mg/kg or more of a subject's body weight. In another embodiment, the dosage of the compositions or antigen binding molecules provided herein administered to prevent, treat, manage, or ameliorate a disorder, or one or more symptom(s) thereof in a subject is 0.1 mg to 200 mg, 0.1 mg to 100 mg, 0.1 mg to 50 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 to 2.5 mg, 0.25 mg to 20 mg, 0.25 to 15 mg, 0.25 to 12 mg, 0.25 to 10 mg, 0.25 mg to 7.5 mg, 0.25 mg to 5 mg, 0.25 mg to 2.5 mg, 0.5 mg to 20 mg, 0.5 to 15 mg, 0.5 to 12 mg, 0.5 to 10 mg, 0.5 mg to 7.5 mg, 0.5 mg to 5 mg, 0.5 mg to 2.5 mg, 1 mg to 20 mg, 1 mg to 15 mg, 1 mg to 12 mg, 1 mg to 10 mg, 1 mg to 7.5 mg, 1 mg to 5 mg, or 1 mg to 2.5 mg.

It may be necessary to use dosages of the compositions or antigen binding molecules outside the ranges disclosed herein in some cases, as will be apparent to those of ordinary skill in the art.

### EXAMPLES

### Example 1: TREM1-PET Imaging of a Mouse Stroke Model (MCAO) Shows that [⁶⁴Cu]TREM1-mAb can Detect Innate Immune Activation in the Brain in Addition to the Peripheral Tissues (Spleen and Gut)

All middle cerebral artery occlusion-reperfusion (abbreviated MCAo) experiments were performed by an experimenter blinded to genotype or pharmacological treatment as described previously (See, Liang X et al. Neuronal and vascular protection by the prostaglandin E2 EP4 receptor in a mouse model of cerebral ischemia. J. Clin. Invest 121, 4362-4371 (2011); and Longa E Z, Weinstein P R, Carlson S & Cummins R Reversible middle cerebral artery occlusion without craniectomy in rats. Stroke 1, 84-91 (1989), which is incorporated by reference herein). The 8-12-week-old male C57BL/6J mice were randomized and subjected to either sham surgery or 45 min of MCA occlusion followed by reperfusion, with survival up to 14 days. Neuroscores were assessed as follows: 0, no deficit; 1, forelimb weakness and torso turning to the ipsilateral side when held by the tail; 2, circling to affected side; 3, unable to bear weight on affected side; 4, no spontaneous locomotor activity or barrel rolling (See, Gelderblom M et al. Temporal and spatial dynamics of cerebral immune cell accumulation in stroke. Stroke 40, 1849-1857 (2009), which is incorporated by reference in its entirety herein, including any drawings).

Conjugation of anti-mouse TREM1-mAb and isotype-contol-mAb (R&D) with DOTA was performed according to standard procedures using metal-free buffers. A solution of DOTA-NHS ester (Macrocyclics Inc.) in dimethyl sulfoxide (25 mmol l⁻¹; 9-12 μl) was added to 1 ml of HEPES buffer (0.1 mol l⁻¹, pH 8.8) containing 500 μg of TREM1-mAb or isotype-control-mAb, and the reaction mixture was incubated at 4° C. overnight. The reaction was quenched with Tris pH 7.4 (Sigma), excess DOTA-NHS was removed by Zeba Spin Desalting Columns (0.5 ml, 70K molecular weight cut-off, ThermoFisher Scientific), and the resulting solution was buffer-exchanged into ammonium acetate buffer (0.1 M, pH 5.5) for ⁶⁴Cu labeling. DOTA-conjugate solutions were concentrated by ultrafiltration (Vivaspin 2 ml, Sartorius) to 1-3 mg ml⁻¹, snap-frozen in liquid nitrogen and stored at −80° C. before radiolabeling. The number of DOTA chelators coupled per antibody molecule was estimated to be between 2 and 4 for both TREM1 and isotype-control, measured via matrix-assisted laser desorption/ionization-time of flight MS, by comparison with unconjugated mAb versus DOTA-conjugated mAb.

Both DOTA-TREM1-mAb and DOTA-isotype-control-mAb were radiolabeled with ⁶⁴Cu (t_{1/2}=12.7 h) using standard methods and metal-free buffers, with some modifications. DOTA-TREM1-mAb/DOTA-isotype-control-mAb (100 μg) in 30-50 μl of 0.25 mol l⁻¹ ammonium acetate buffer (0.1 M, pH 5.5) was mixed with pH-balanced ⁶⁴CuCl₂ solution (44-74 MBq, pH 4.5-5.0, University of Wisconsin) at 37° C. with gentle shaking at 400 r.p.m. After a 30-60 min incubation period, 0.1 M EDTA (0.5 M, pH 8.0) was added to a final concentration of 0.01 M and incubated at 22° C. for 15 min to scavenge unchelated [⁶⁴Cu]CuCl₂ in the reaction mixture.

Purification of each radiolabeled antibody was achieved by G25 Sephadex size-exclusion purification (NAP-5 column). Radiochemical purity was determined by instant thin-layer chromatography with TEC-Control Chromatography strips (Biodex Medical Systems), developed in saline, and size-exclusion liquid chromatography with a Phenomenex SEC 3000 column (Torrance) with sodium phosphate buffer (0.1 mol l⁻¹, pH 6.8) at a flow rate of 1.0 ml min⁻¹. ⁶⁴Cu-labeled anti-TREM1-mAb (that is, [⁶⁴Cu]TREM1-mAb) and ⁶⁴Cu-labeled isotype-control mAb (that is, [⁶⁴Cu]ISO-mAb) were obtained with high specific radioactivity (>0.400 MBq μg⁻¹), radiochemical purity (>99%) and labeling efficiency (70-95%) and formulated in phosphate-buffered saline (0.1 mol l⁻¹ NaCl, 0.05 mol l⁻¹ sodium phosphate (pH 7.4)).

T2-weighted structural MRI images were acquired 1.0-1.5 d post-MCAo surgery to confirm successful stroke and provide anatomical reference for PET image analysis. Images were acquired using a 7 T MRI Varian Magnex Scientific MR scanner system and a millipede quadrature radiofrequency coil.

MCAo and sham mice were injected with [⁶⁴Cu]TREM1-mAb (1.31-4.38 MBq) or [⁶⁴Cu]ISO-mAb (1.59-3.63 MBq) intravenously. PET tracer was injected 12 h after MCAo, and mice were imaged 3 h and 24 h later. PET images acquired at 3 hours were not as useful as those acquired at 24 hours since antibody-PET tracers have a long blood residence and high levels of unbound tracer in blood can obscure visualization of bound tracer in tissues. An antibody-PET tracer must sufficiently clear from blood before imaging to achieve high signal-to-background images.

Mice were then imaged at 19-20 hours post intravenous injection. Mice were anesthetized using isoflurane gas (2.0-3.0% for induction and 1.5-2.5% for maintenance). A CT image was acquired immediately before each PET scan. CT raw images were acquired at 80 kVp at 500 μA, two-bed position, half-scan 220° of rotation and 120 projections per bed position with a cone beam micro-X-ray source (50 μm focal spot size) and a 2,048 pixel×3,072 pixel X-ray detector. On the basis of attenuation correction from the CT measurements, each 10 minute static PET scan was acquired with default settings of coincidence, a timing window of 3.4 ns and an energy window of 350-650 keV. PET and CT image files were co-registered and analyzed using Inveon Research Workspace software (IRW, v.4.0; Siemens). PET images were reconstructed with the three-dimensional ordered subsets expectation maximization (OSEM3D) algorithm.

The PET system can deliver ˜1.5-2.0-mm spatial resolution, and a maximum field of view of 10 cm×30 cm. OSEM3D/maximum a posteriori (MAP) reconstruction yields uniform spatial resolution in all directions, with an average full width at half maximum of 1.656±0.06 mm. All PET images were reconstructed using two iterations of OSEM3D algorithm (12 subsets) and 18 iterations of the accelerated version of 3D-MAP (that is, FASTMAP)-matrix size of 128×128×159.

PET, CT, and brain MR image files were co-registered and analyzed with VivoQuant (VQ, v. 2.0, inviCRO) and IRW software (v.4.0). Regions of interest (ROIs) were drawn around the infarct using the MR image as a guide and then copied to the contralateral hemisphere using VQ software, while peripheral organ ROIs were drawn using IRW. The mean concentration of radioactivity contained within each ROI (Bq cm⁻³) was used to calculate percentage injected dose (ID) per g (% ID g⁻¹) values, using the decay-corrected dose for each mouse at the time of the PET scan.

Following the final PET scan, mice were deeply anesthetized with 2-2.5% isoflurane. Blood samples (100-200 μl) were collected via cardiac puncture immediately before transcardial perfusion using 20-30 ml of PBS. All mice that underwent PET imaging were euthanized after perfusion with saline to remove possible unbound intravascular [⁶⁴Cu]TREM1-mAb. Blood, heart, liver, lungs, spleen, left brain, and right brain hemispheres were extracted/dissected from each mouse, placed in a tube for gamma counting and weighed. Satisfactory perfusions were verified by visual inspection of brain tissue. Dissected tissues were then counted via an automated gamma counter (Cobra II Auto-Gamma counter; Packard Biosciences Co.) and tissue-associated radioactivity was then normalized to tissue weight and to the amount of radioactivity administered to each mouse, and decay-corrected to time of tracer injection using diluted aliquots of the initial administered dose as standards.

At 20 hours post-injection of radiotracer, n=3 mice injected with [⁶⁴Cu]TREM1-mAb (3.34-7.88 MBq) and n=3 mice injected with [⁶⁴Cu] isotype-control-mAb (2.90-7.97 MBq) were deeply anesthetized using isoflurane gas (2.0-3.0%) and perfused with 30-0 ml of PBS. Brain tissue was quickly embedded in optimal cutting temperature compound (Tissue-Tek) and coronal sections (20 μm) were obtained for ex vivo autoradiography. Autoradiography was conducted and the anatomy of brain sections was confirmed by Nissl staining (cresyl violet acetate; Sigma Aldrich) using standard techniques. 20-μm thick sections were mounted on microscope slides (Fisherbrand Superfrost Plus Microscope Slides), air-dried for 10 minutes, and then exposed to a high-resolution digital storage phosphor screen (GE Lifesciences) for 72 hours at −20° C. Ex vivo autoradiography images of brain sections were quantified by drawing ROIs around the infarct using Nissl staining to verify. The digital storage phosphor screen was scanned using a Typhoon 9410 Variable Mode Imager (Amersham Biosciences) and images were analyzed using ImageJ (image processing and analysis software in Java, v.1.45s).

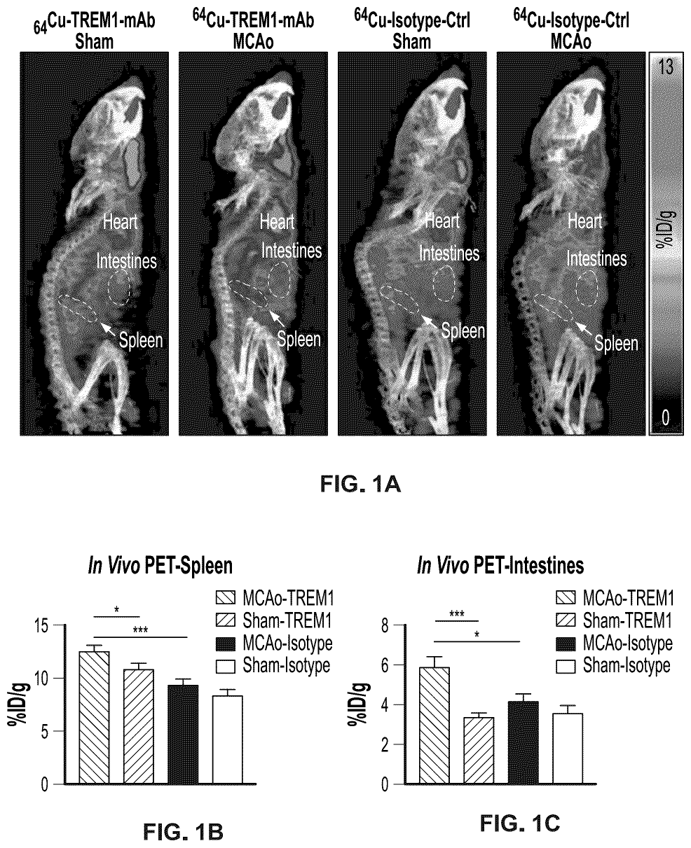

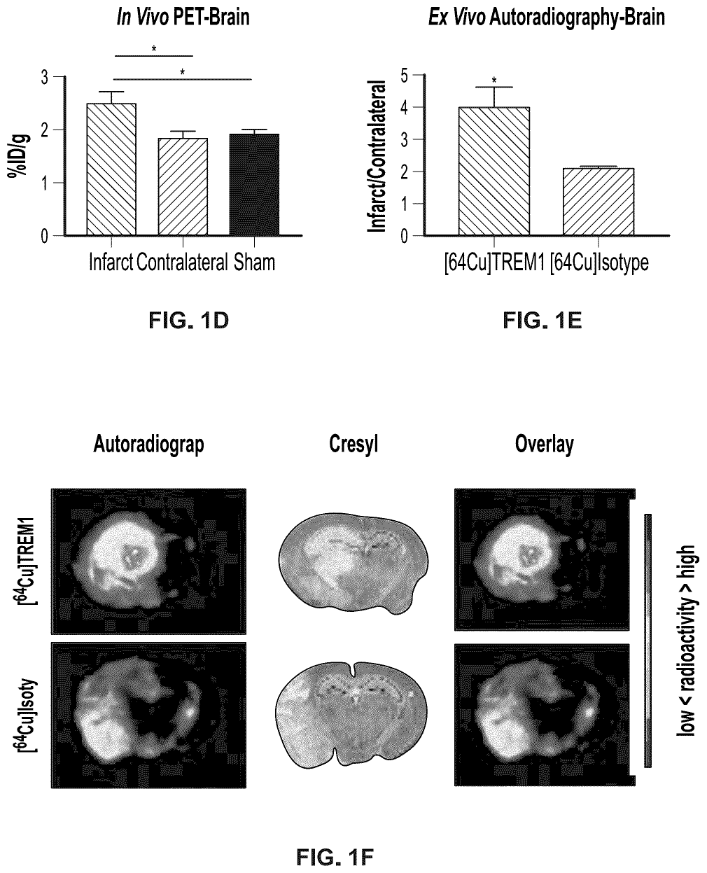

Results can be seen in FIGS. 1A-1F. The images on the top left in FIG. 1A show 3D sagittal maximum intensity projection PET/CT images of representative sham and MCAo mice, 1.5-2 days post-surgery, injected with either [⁶⁴Cu]TREM1-mAb or [⁶⁴Cu]Isotype-control-mAb. The images directly below in FIG. 1B and FIG. 1C that show quantitation of in vivo PET signal in spleen and intestines, respectively, and demonstrates higher uptake of [⁴Cu]TREM1-mAb in MCAo mice (n=12) compared to sham mice (n=10) and that the signal specifically reflects TREM1 due to significantly less signal in the same tissues of MCAo (n=8) and sham mice (n=3) injected with [⁶⁴Cu]Isotype control-mAb. The images on the top right in FIG. 1D shows quantitation of brain signal from in vivo PET images and ex vivo autoradiography, respectively. As can be seen, in vivo quantitation reveals significantly higher [⁶⁴Cu]TREM1-mAb signal within the infarct of MCAo mice, compared to uptake in a corresponding contralateral brain region and also uptake in an equivalent brain region from Sham mice (n=9 per group). Also, as can be seen, FIG. 1E shows ex vivo biodistribution of brain hemispheres, following removal of unbound intravascular tracer (i.e., perfusion), corroborates brain PET quantitation (n=9 MCAo, n=10 Sham). ****=p<0.001, ***=p<0.005, **=p<0.01, *=p<0.05. FIG. 1F Representative autoradiography images of coronal brain sections, cresyl violet staining and overlay of autoradiography and cresyl staining from mice imaged with [⁶⁴Cu]TREM1-mAb or [⁶⁴Cu]isotype-control-mAb 36 h after MCAo. FIG. 1 shows that TREM1 is a sensitive and specific PET imaging biomarker that crosses the blood brain barrier and enables detection of myeloid cell-driven immune responses in the CNS and periphery after ischemic stroke.

### Example 2: Tracking Peripheral Infiltrating Activated Myeloid Cells with TREM1-PET in a Mouse Model of Chronic Multiple Sclerosis

Wild-type (WT) and TREM1 KO mice were induced with experimental autoimmune encephalomyelitis (“EAE”) using MOG₃₅₋₅₅ emulsified in immune adjuvant and subsequently grouped by disease severity. EAE is the most commonly used experimental/preclinical model for multiple sclerosis. EAE is a complex condition in which the interaction between a variety of immunopathological and neuropathological mechanisms leads to an approximation of the key pathological features of MS: inflammation, demyelination, axonal loss, and gliosis.

Mice were categorized as pre-symptomatic (pre; score 0, >1 g weight loss in 48 h), low (score 0.5-2), or high EAE (score 2.5-4.5). Anti-TREM1 monoclonal antibody (mAb) was DOTA-conjugated and radiolabeled with ⁶⁴Cu. PET/CT imaging was performed 20 hours post-injection of [⁶⁴Cu]TREM1-mAb (95-120 μCi, >99% RCP) or 50-60 min after TSPO-targeted radiotracer [¹⁸F]GE-180 (231-269 μCi, >99% RCP). Following PET, mice were perfused to remove unbound intravascular tracer and radioactivity in central nervous system (CNS) tissues was measured using a gamma counter.

Spinal cords were further analyzed via high-resolution autoradiography. Flow cytometry was performed on CNS tissues using TREM1, CD45, CD11b, CD11c, CD3, and Ly-6G to delineate immune cell populations. Quantitative PCR was performed to assess changes in mRNA expression. LP17, a decoy receptor peptide known to attenuate TREM1 signaling, was administered daily to pre EAE mice (10, 15 mg/kg, or saline i.p) for 10 days.

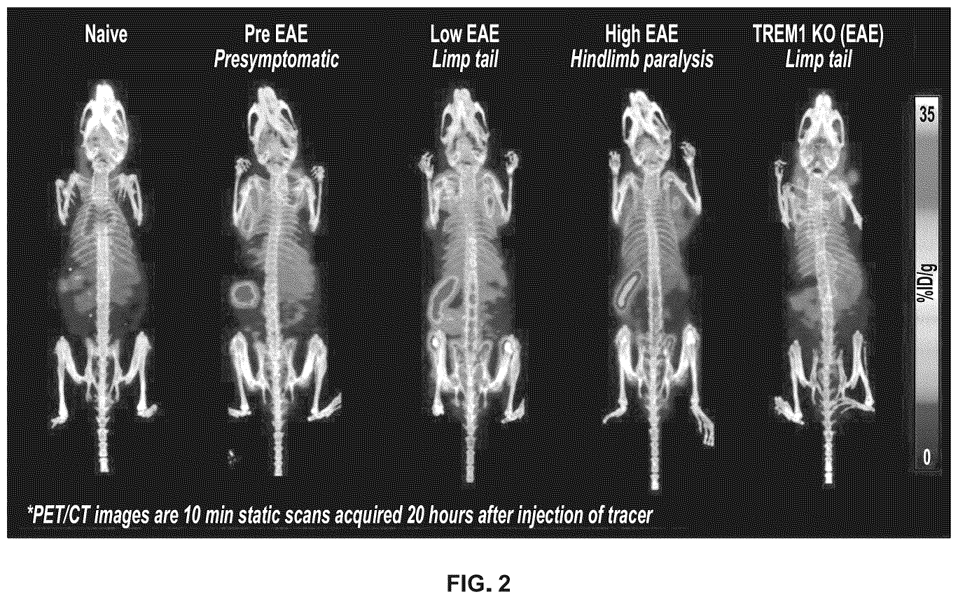

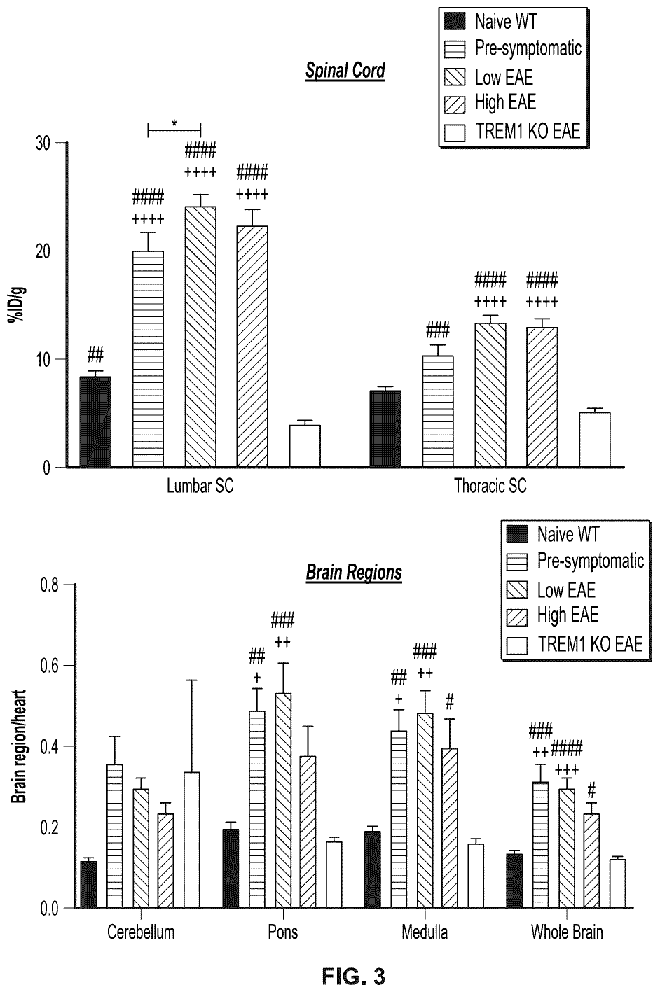

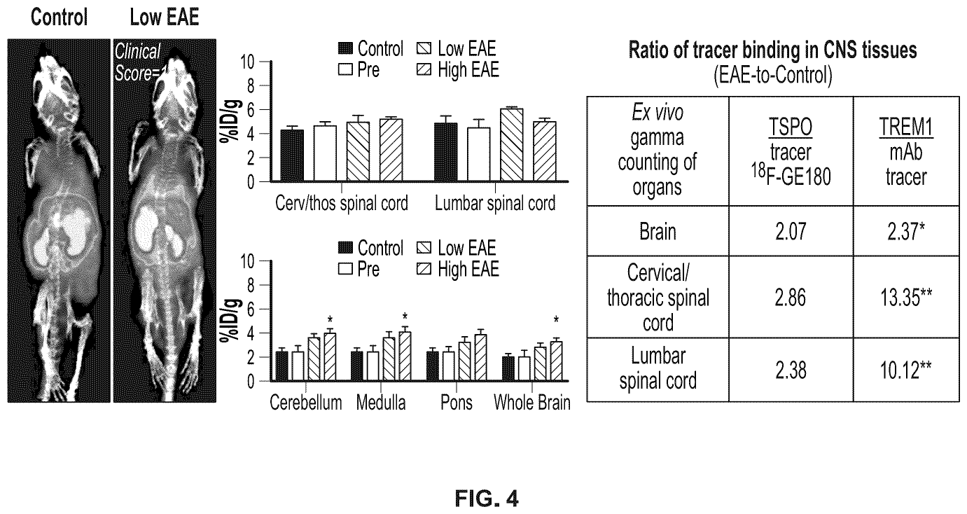

The results can be seen in FIG. 2, FIG. 3, and FIG. 4. In FIG. 2, the left image shows naive mice; the next image over labeled Pre-EAE shows a presymptomatic mouse; the middle image shows a low EAE mouse with a limp tail; the image further right shows a mouse with high EAE and hind limb paralysis; and the image furthest to the right shows a TREM1 EAE knockout mouse, also with a limp tail. PET/CT images are 10 minute static scans acquired 20 hours after injection of the tracer.

FIG. 3 displays results quantification of TREM-1 PET signal for spinal cord and brain regions. The left side of FIG. 3 shows spinal cord (SC), with the left graph showing lumbar SC and the right graph showing thoracic SC. The right side of FIG. 3 shows the brain regions, labelled appropriately along the bottom as cerebellum, pons, medulla, and whole brain. As can be seen from the comparison of naive, pre-symptomatic, low EAE, high EAE, and TREM1 KO EAE, TREM1-PET detects pro-inflammatory peripheral CNS-infiltrating myeloid cells in EAE.

FIG. 4 displays comparative results for detecting toxic inflammation in EAE using either TREM1-PET or TSPO-PET. The left side shows control and low EAE. The left side of the top middle shows control, pre, low EAE, and high EAE for cervical/thoracic spinal cord and the right side of the top middle shows control, pre, low EAE, and high EAE for lumbar spinal cord. The bottom shows control, pre, low EAE, and high EAE for brain regions: cerebellum, medulla, pons, and whole brain. The chart on the right side shows the ratio of tracer binding in CNS tissues (EAE/control) for brain, cervical/thoracic spinal cord, and lumbar spinal cord. TREM1-PET is more sensitive than TSPO-PET at detecting toxic inflammation in EAE.

### Example 3: TREM1-PET of a Relapsing Remitting MS Mouse Model (RR-EAE)

SJL mice were induced with relapsing-remitting experimental autoimmune encephalomyelitis (RR-EAE) using PLP₁₃₉₋₁₅₁ emulsified in immune adjuvant. Mice during active EAE disease (exhibiting paresis and/or paralysis) and remission (exhibiting complete recovery following initial EAE symptoms, similar to what occurs in RR-MS patients) were used. Anti-TREM1 monoclonal antibody (mAb) was DOTA-conjugated and radiolabeled with ⁶⁴Cu. PET/CT imaging was performed 20 h post-injection of [⁶⁴Cu]TREM1-mAb (95-120 μCi, >99% RCP). Following PET imaging, mice were perfused with saline to remove any unbound intravascular tracer and spinal cords were analyzed via high-resolution autoradiography.

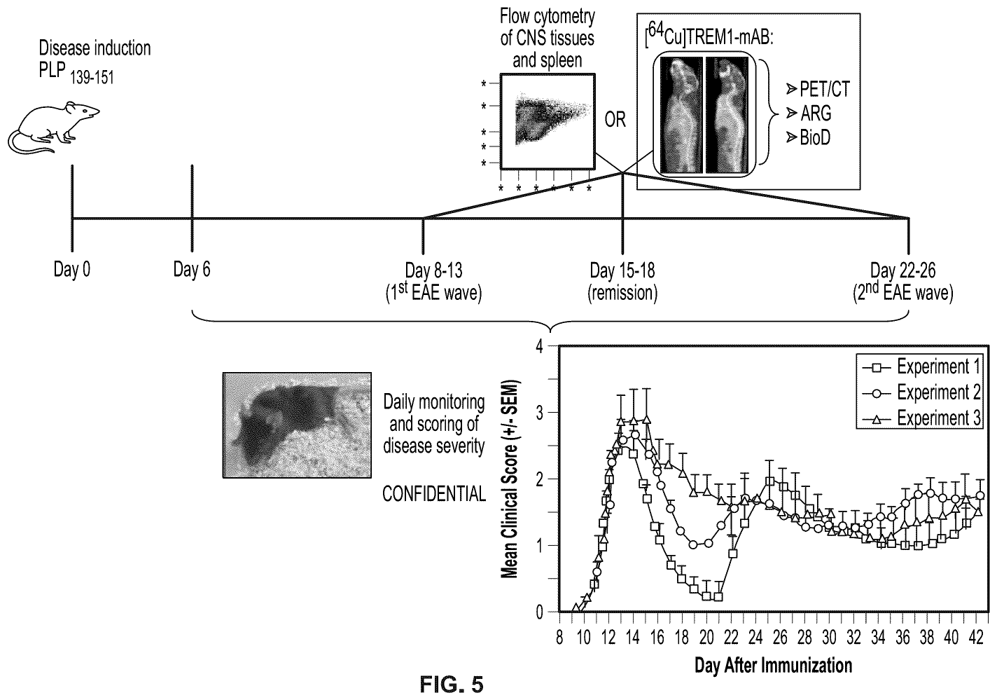

FIG. 5 shows the study design for assessing TREM1-PET as a tool to monitor disease in RR-EAE. Disease is induced and mice are monitored and scored daily for disease severity. A first EAE wave occurs between day 8 and day 13. Remission occurs between day 15 and day 18. A second EAE wave occurs between day 22 and day 26.

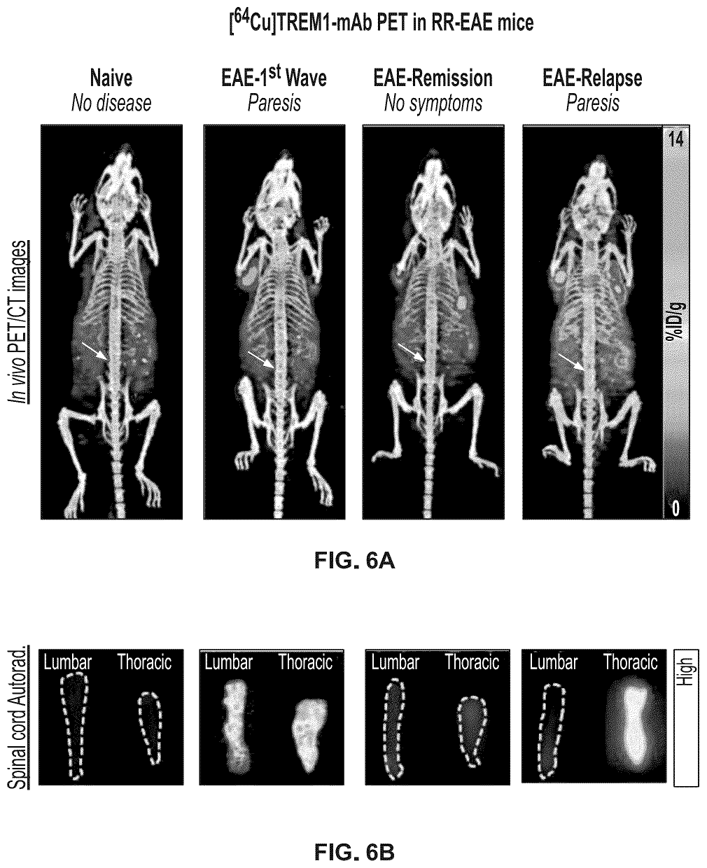

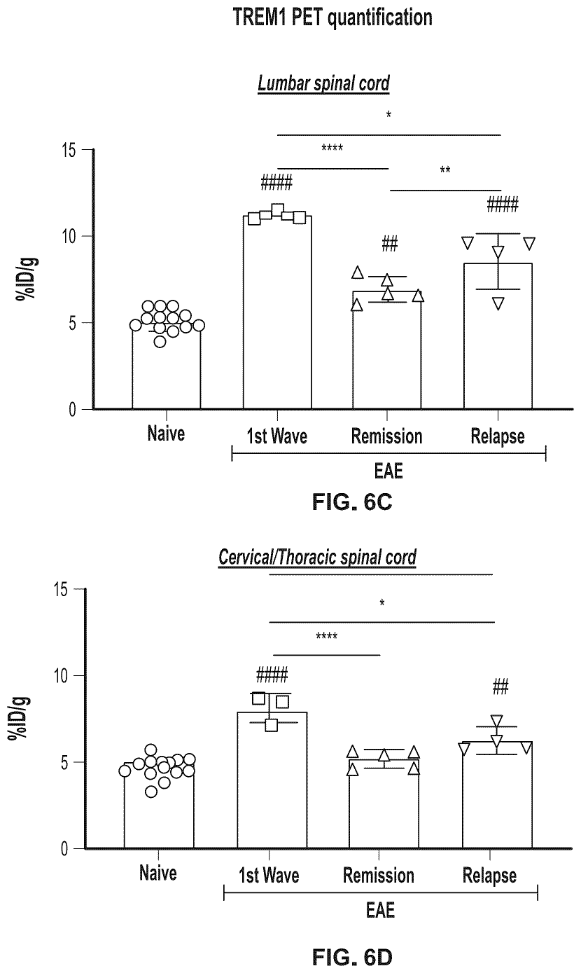

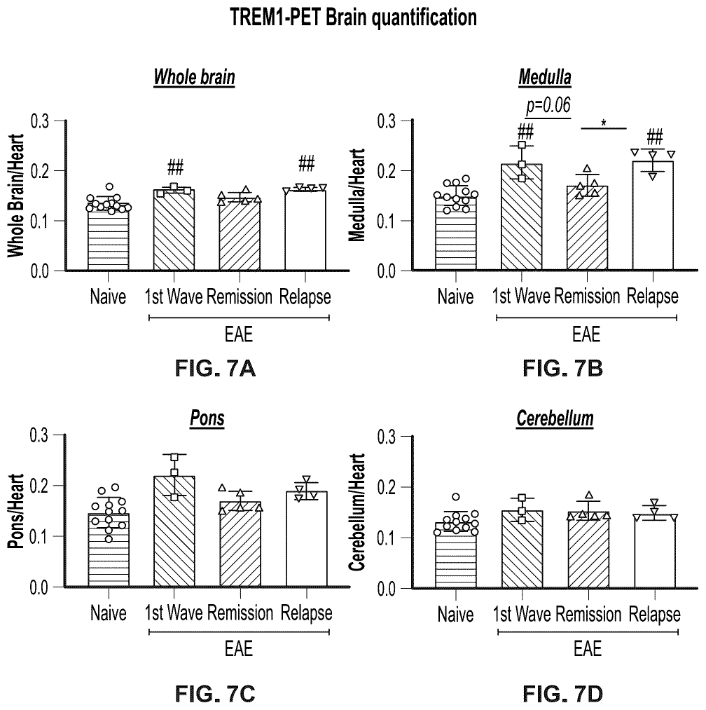

Results can be seen in FIG. 6, and in FIG. 7. The left side of FIG. 6 shows [⁶⁴Cu]TREM1-mAb PET in RR-EAE mice, with the top showing in vivo representative PET/CT images for mice in the naive group, the first EAE wave, EAE remission, and then EAE relapse and the bottom showing lumbar and thoracic spinal cord autoradiography for a naive, the first EAE wave, EAE remission, and then an EAE relapse mouse. The top right side of FIG. 6 shows TREM1 PET quantification for naive, the first EAE wave, EAE remission, and EAE relapse mice for lumbar spinal cord. The bottom right side of FIG. 6 shows TREM1 PET quantification for naive, the first EAE wave, EAE remission, and then EAE relapse mice for cervical/thoracic spinal cord.

FIG. 7 shows TREM1-PET brain quantification. The top left side of FIG. 7 shows TREM1 PET quantification for naive, the first EAE wave, EAE remission, and EAE relapse mice for the whole brain. The top right side of FIG. 7 shows TREM1 PET quantification for naive mice, the first EAE wave, EAE remission, and then an EAE relapse for the medulla. The bottom left side of FIG. 7 shows TREM1 PET quantification for naive mice, the first EAE wave, EAE remission, and then an EAE relapse for the pons. The bottom right side of FIG. 7 shows TREM1 PET quantification for naive mice, the first EAE wave, EAE remission, and then an EAE relapse for the cerebellum. FIG. 6 and FIG. 7 show that TREM1-PET imaging agent [⁶⁴Cu]TREM1-mAb crosses the blood brain barrier and provides sensitive monitoring of relapses and remissions in EAE.

### Example 4: TREM1-PET Imaging of a Mouse Model of LPS-Induced Septic Shock Demonstrated the Ability of Our Imaging Agent to Cross the BBB and Detect Subtle Inflammation in the Brain

Anti-TREM1 monoclonal antibody (mAb) was DOTA-conjugated and subsequently radiolabeled with ⁶⁴Cu. Static PET/CT images were acquired 20 hours post IV administration of [⁶⁴Cu]TREM1-mAb (90-100 μCi) to 1) wild-type (wt) mice following IP injection of 5 mg/kg LPS (LPS-wt), 2) saline-treated wt mice (vehicle-wt), and 3) LPS-treated TREM1 knockout mice (LPS-KO). [⁶⁴Cu]-labeled isotype-control-mAb was administered to wt mice following IP injection of 5 mg/kg LPS (LPS-ISO-wt) to evaluate specificity of [⁶⁴Cu]TREM1-mAb. After perfusion of mice, organs were dissected and radioactivity was measured with a gamma counter to corroborate PET findings. Quantitative analysis of PET images was performed by drawing 3D volume region-of-interest (ROI) on tissues of interest such as spleen, lung, liver, and brain regions. Flow cytometry of spleens and brains was performed to correlate PET signal with levels of TREM1 positive myeloid cells.

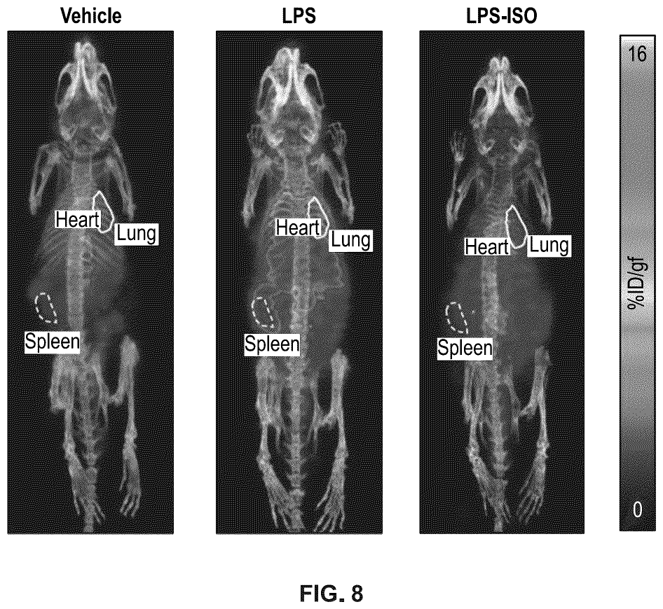

FIG. 8 shows TREM1-PET is a specific tool for detecting activated myeloid cells after LPS challenge. Representative 3D maximum intensity projection images are depicted. The left image shows a C57/BL6 wild-type mice treated with vehicle (Vehicle-wt); the middle image shows a wild-type mice treated with LPS (LPS-wt); and the right image shows wt mice treated with LPS and imaged with a radiolabeled isotype control antibody (LPS-ISO-wt). The pattern of [⁴Cu]TREM1-mAb uptake in LPS-wt mice corresponds with the increase in splenic myeloid cells, relative to vehicle treated mice, known to occur after LPS challenge and also with inflammation in the lungs and liver, which are among the first organs to be inflamed in LPS-induced sepsis. Low activity in the spleen, liver, and lung of LPS-wt mice injected with [⁶⁴Cu]isotype control-mAb confirms the specificity of the TREM1 tracer.

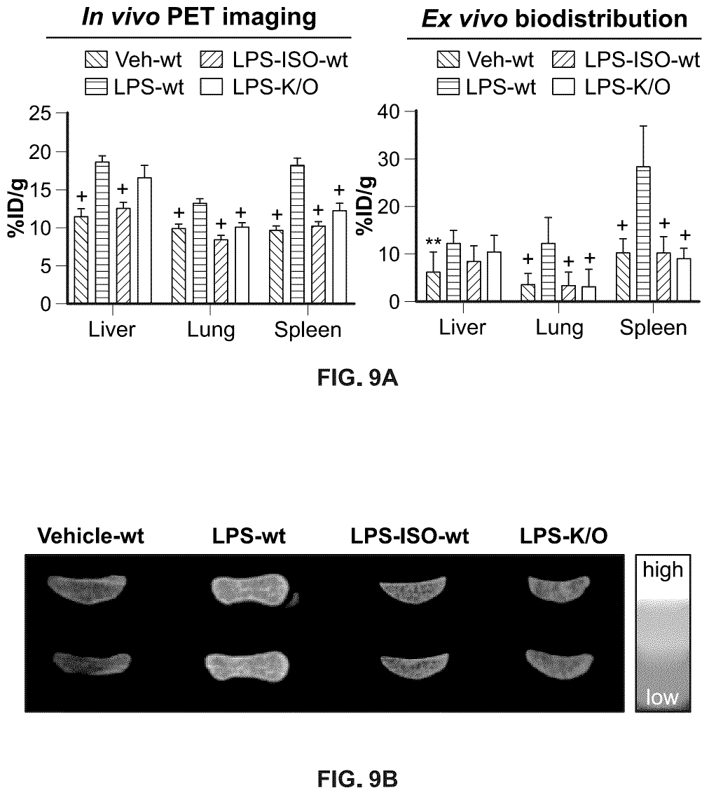

FIG. 9 further demonstrates that TREM1-PET is a specific tool for detecting activated myeloid cells after LPS challenge. The top left shows quantitative analysis of in-vivo PET imaging and the top right shows ex-vivo bio distribution (20h post-injection of tracer), each of which corroborates PET imaging findings. The bottom shows a spleen autoradiography (20 hours post injection) which shows significantly increased uptake in LPS-wt spleen compared to vehicle (Vehicle-wt), isotype (LPS-ISO-wt), and knockout mice (LPS-KO). (*=<0.05, **=<0.01, +=<0.005, vs. LPS-wt) (n=5-9/group).

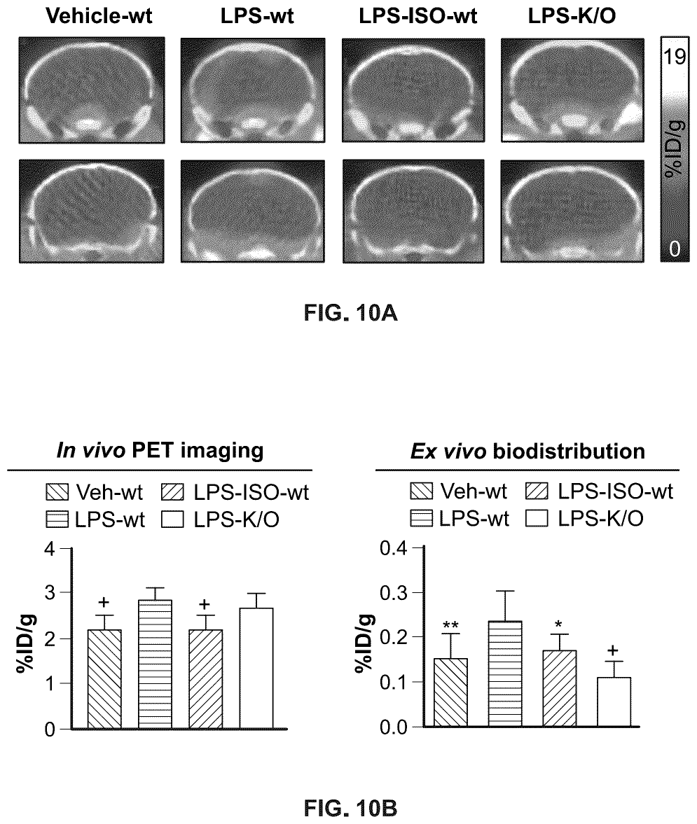

FIG. 10 shows TREM1-PET imaging reveals subtle neuroinflammation in a mouse model of sepsis and uptake of the TREM-1 antibody in the brain. Top (a) representative coronal PET/CT images show increased brain uptake for the antibody for the LPS-wt group compared to Veh-wt, LPS-K/0, and LPS-wt mice imaged with [⁶⁴Cu]isotype control-mAb (LPS-ISO-wt). Bottom (b) shows quantification of brain uptake observed in PET images and through ex vivo gamma counting of whole brain tissue (*=<0.05, **=<0.01, +=<0.005, vs. LPS-wt) (n=5-9/group), with the left side showing in vivo PET imaging and the right side showing ex vivo biodistribution. FIG. 10 demonstrates the delivery of one or more substance(s) to the brain using the TREM-1 antibody.

### Example 5: TREM1-PET Imaging of Two Alzheimer's Disease Mouse Models Reveals Elevated Signal in the Brain Parenchyma, Choroid Plexus, and Ventricles Compared with Age/Sex-Matched Wild-Type Controls

TREM1-specific mAb was radiolabeled with ⁶⁴Cu (See, Liu Q. et al., Peripheral TREM1 responses to brain and intestinal immunogens amplify stroke severity, Nature Immunology, August; 20(8): 1023-1034, (2019), which is incorporated by reference herein in its entirety). The radiolabeled antibody ([⁶⁴Cu]-TREM1-mAb) was administered to two familial AD (FAD) models, the 5×FAD mouse model and the APPSwe (TG2576) mouse model, in addition to age/sex-matched wild-type littermates.

5×FAD transgenic mice develop AD pathogenesis rapidly in their brains, with amyloid plaques appearing in the hippocampus beginning at 3 and 4 months of age (See, Oakley, H. et al. Intraneuronal beta-amyloid aggregates, neurodegeneration, and neuron loss in transgenic mice with five familial Alzheimer's disease mutations: potential factors in amyloid plaque formation. J. Neurosci. 26, 10129-10140 (2006), which is incorporated by reference in its entirety herein). The 5×FAD model rapidly develops severe amyloid pathology. These mice accumulate high levels of intraneuronal Aβ42, beginning around 1.5 months of age. Extracellular amyloid deposition begins around 2 months, first in the subiculum and layer V of the cortex, and increase rapidly with age. Plaques are found throughout the hippocampus and cortex by six months; in older mice, plaques are present in the thalamus, brainstem, and olfactory bulb, but are absent from the cerebellum.

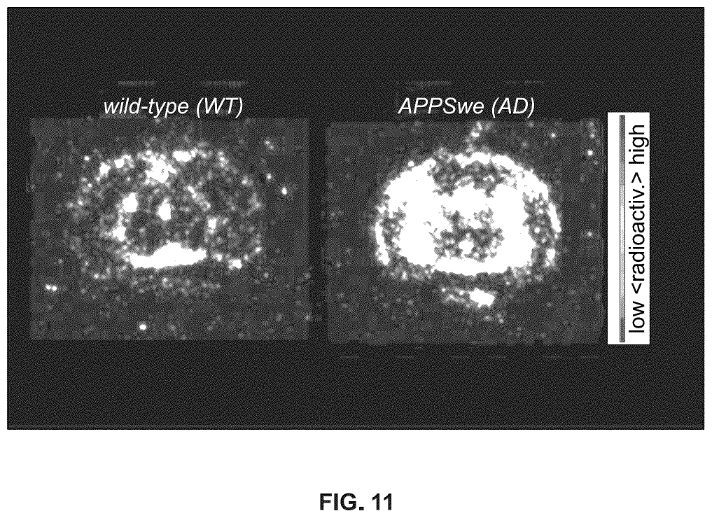

Mice were imaged 20 hours after injection of [⁶⁴Cu]TREM1-mAb using PET/CT. FIG. 11 shows results in the 5×FAD model. [⁶⁴Cu]TREM1-mAb PET signal was significantly elevated in the hippocampus of 6 month 5×FAD mice compared to wild-types as shown by in vivo PET imaging on the top left side and on the right side. Immediately following PET, all mice were perfused to remove any unbound intravascular tracer. Brains were dissected and sectioned (40 μm-thick coronal hemi-brain slices) for ex vivo autoradiography to confirm PET findings.

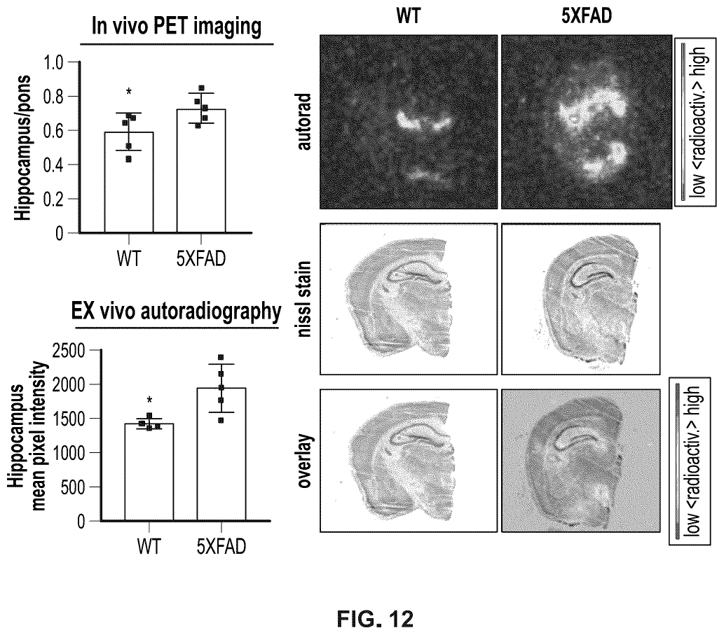

The bottom left of FIG. 12 (autoradiography overlaid with Nissl staining) shows an increased [⁶⁴Cu]TREM1-mAb PET signal by ex vivo autoradiography in 5×FAD mice as compared to wild type mice. The results show that [⁶⁴Cu]TREM1-mAb PET signal was significantly elevated in the hippocampus of 6-month 5×FAD mice compared to wild-types.

PET signal was also significantly elevated in the APPSwe (TG2576) model as compared to age matched littermate control mice. APPswe/PS1dE9 mice overexpress the Swedish mutation of APP, together with PS1 deleted in exon 9 (See Jankowsky J L, Fadale D J, Anderson J, et al. Mutant presenilins specifically elevate the levels of the 42 residue β-amyloid peptide in vivo: evidence for augmentation of a 42-specific γ secretase. Human Molecular Genetics. 2004; 13(2):159-170, which is incorporated by reference herein in its entirety). These mice develop the first Aβ plaques at 4 months of age. Even though these mice do not exhibit frank neuronal loss, the APPswe/PS1dE9 mice display a variety of other clinically relevant AD-like symptoms.

FIG. 11 shows elevated TREM1-PET signal in the brain of APPSwe mice (right side) as compared to age-matched wild-type mice (left side). In both the 5×FAD model and the APPSwe model, [⁶⁴Cu]TREM1-mAb PET signal was evident in the hippocampus, cerebral cortex, and choroid plexus.

These findings indicate that the radiolabeled TREM1 antibody is able to cross the blood brain barrier (BBB). Taken together, these PET and confirmatory autoradiography findings demonstrate presence of radiolabeled TREM1 antibody in brain parenchyma in FAD mice but not wild type control littermates. These findings provide proof that [⁶⁴Cu]TREM1-mAb signal can be detected in preclinical FAD models to assess spatial distribution of disease modifying myeloid inflammation.

### Example 6: Whole Body Imaging of Maladaptive Myeloid Cell Activation Using TREM-1 PET

Anti-TREM1 monoclonal antibody (mAb) was DOTA-conjugated and radiolabeled with copper-64 (⁶⁴Cu). Static PET/CT images were acquired 3-40 hours after intravenous administration of ⁶⁴Cu-TREM1-mAb to wild-type (WT) mice treated with 5 mg/kg LPS (LPS-Wr) or vehicle alone (Veh-Wr). Gamma counting and autoradiography were conducted to confirm in vivo findings. RT-qPCR and flow cytometry were performed to assess alterations in TREM1 expression and cellular specificity in different tissues from LPS-WT versus Veh-Wr mice. Luminex was used to investigate the relationship between TREM1-PET signal and inflammatory plasma cytokine signatures. Finally, the effect of genetically knocking out TREM1 on sickness behavior in LPS-injected mice was tested via survival studies and murine sepsis scoring.

Quantification of TREM1-PET images revealed significantly higher signal in organs known to be affected by LPS challenge (brain, liver, lung, and spleen: p<0.01 vs Veh-WT), which was confirmed by ex vivo gamma counting and autoradiography. The specificity of ⁶⁴Cu-TREM1-mAb was verified by its significantly lower binding in the brain, lungs, and spleen of LPS-treated-TREM1 knockout mice (LPS-K/O) versus LPS-WT mice (p<0.01), in addition to the relatively lower binding of ⁶⁴Cu-Isotype-control in LPS-treated Wr mice (LPS-ISO-WT). Flow cytometry demonstrated significant increases in TREM1⁺ myeloid cells in the brain, lungs, and spleen of LPS-Wr versus Veh-WT mice (p<0.01-p<0.0001), which was corroborated by RT-qPCR. Furthermore, TREM1-PET signal correlated with pro-inflammatory cytokine signatures and decreased survival of LPS-injected mice, all as follows.

To identify the optimal time-point for imaging this model (i.e., the time post-tracer injection that affords the highest signal-to-background images), serial 10-minute static TREM1-PET imaging was performed at 3, 20, and 40 hours post-injection (hpi) of tracer, in a small cohort of wild-type (WT) mice that received an intraperitoneal (i.p.) injection of 5 mg/kg LPS (LPS-WT) or saline (vehicle [Veh]-WT). Tracer was injected 4-5 hours after mice received LPS or saline. Both systemic- and neuro-inflammation have been reported in this mouse model as early as 1 hour following i.p. LPS and maintained for at least 72 hours, with some reports that brain TNF-α levels remain elevated for months after a single i.p. injection. Following final PET/CT imaging with ⁶⁴Cu-TREM1-mAb or a ⁶⁴Cu-labeled isotype-control mAb (⁶⁴Cu-Isotype-control-mAb, used to assess specificity of the TREM1-PET tracer), ex vivo gamma counting of tissues and high-resolution autoradiography were conducted to confirm in vivo findings at the optimal imaging time-point. RT-qPCR was performed on mice from a separate cohort to assess levels of TREM1 in tissues known to be inflamed following LPS challenge (i.e., brain, liver, lungs, spleen) between 3-72 hours. Flow cytometry was carried out using LPS-WT, Veh-WT, and TREM1-knockout (K/O) mice administered LPS (LPS-K/O) mice to validate TREM1 as a specific marker of innate immune activation in the brain, lung, and spleen. The relationship between TREM1-PET signal and peripheral inflammatory plasma cytokine signatures was investigated using a bead-based immunoassay (i.e., Luminex). Finally, the effect of attenuating TREM1 signaling via genetic K/O was studied by comparing the murine sepsis score and survival of WT compared to TREM1-K/O mice administered with LPS.

Female C57BL/6 WT and TREM1-K/O mice (8-12 weeks, original breeders provided by Dr. Christoph Mueller, University of Bem) were housed under a 12-hour light/dark schedule with adlibitum food and water access. LPS (Escherichia coli lyophilized powder; Sigma) was dissolved in sterile saline immediately prior to i.p. injection (5 mg/kg). Veh-WT mice received equivalent volumes (by weight) of sterile saline.

Conjugation of anti-mouse anti-TREM1-mAb and isotype-control-mAb (R&D, IgG_{2A}) with 1,4,7,10-tetraazacyclododecane-1,4,7,10-tetraacetic acid (DOTA) and subsequent radiolabeling with ⁶⁴Cu (half-life: 12.7 hours) was performed using standard procedures and metal-free buffers. ⁶⁴Cu-TREM1-mAb and ⁶⁴Cu-Isotype-control were obtained with high molar activity (>0.400 MBq/μg), labeling efficiency (70-99%), and >99% radiochemical purity.

LPS-WT, Veh-WT, and LPS-K/O mice were injected with 3.3-4.4 MBq of ⁶⁴Cu-TREM1-mAb or ⁶⁴Cu-Isotype-control-mAb (both formulated in phosphate-buffered saline) intravenously (i.v.) 4 hours following LPS injection. PET/CT images were acquired 19-20 hpi using a dual PET/CT scanner (Inveon; Siemens). Static PET images (10-minutes) were reconstructed using a 3-dimensional ordered subsets expectation maximization algorithm.

PET and CT images were co-registered using Inveon Research Workplace image analysis software (v4.2; Siemens) and CT images were used to manually determine liver, lung, and spleen regions of interest (ROIs). Brain PET quantification was performed using a semi-automated brain atlas approach in VivoQuant. PET data is expressed as percent injected dose per gram (% ID/g).

Following PET, a blood sample was collected from each mouse via cardiac puncture immediately prior to transcardial perfusion. After perfusion, the heart, lungs, liver, spleen, kidney, and brain were dissected from each mouse, and gamma counting was performed using a Cobra II Auto-Gamma counter (Packard Biosciences Co.) to quantify % ID/g. Ex vivo high-resolution autoradiography was performed using 40 μm-thick brain and spleen sections. These sections were subsequently stained with cresyl violet (Sigma Aldrich) and hematoxylin and eosin (H&E, Fisher Scientific) to visualize regional tracer binding in brain and spleen respectively.

Single cell suspensions were obtained from brain, lung, and spleen via mechanical homogenization following transcardial PBS perfusion. Live myeloid, lymphoid, and astrocyte populations were stained prior to 2% paraformaldehyde (ChemCruz) fixation and analyzed using FlowJo software (Tree Star Inc.).

Blood collected in EDTA-coated tubes (BD) was centrifuged (400-500 RCF, 10 minutes). Resulting plasma samples were analyzed by the Stanford Human Immune Monitoring Core using a 38-plex murine-specific Luminex array (eBiosciences/Affymetrix).

A concentration of 15 mg/kg LPS was selected as the dose for all survival studies after obtaining pilot study results assessing morbidity rate following single injection of 5, 10, 15, or 20 mg/kg LPS. A single dose of 5 or 10 mg/kg did not lead to any morbidity within 24-48 hours while 20 mg/kg had very potent effects of >50% morbidity within 24 hours; 15 mg/kg was closest to, without exceeding, the dose that leads to death of 50% of mice (i.e., LD50). Female TREM1-K/O mice and WT littermates (20-23 weeks) were injected i.p. with LPS (15 mg/kg of LPS dissolved in saline). Mice were monitored daily for a week, and appearance (coat smoothness and piloerection) and activity level (natural or when provoked) assessed using a numerical murine sepsis severity scoring system.

GraphPad Prism (v9.01) was used to perform statistical analyses of flow cytometry, in vivo PET and ex vivo gamma counting data; R (v3.3.3) was used for cytokine analysis. All data was assessed for normalization, and parametric and non-parametric tests were applied as appropriate. A p-value≤0.05 was considered significant.

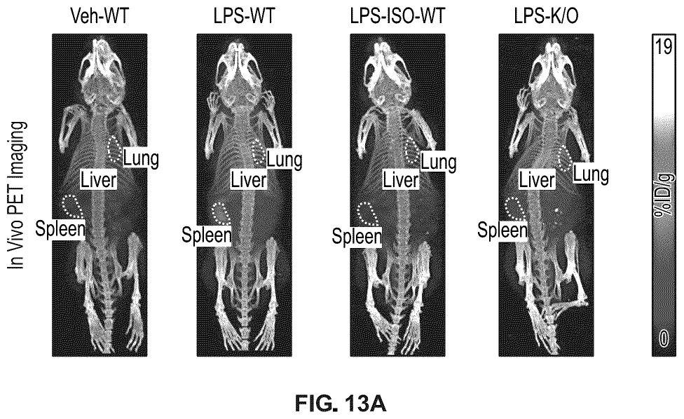

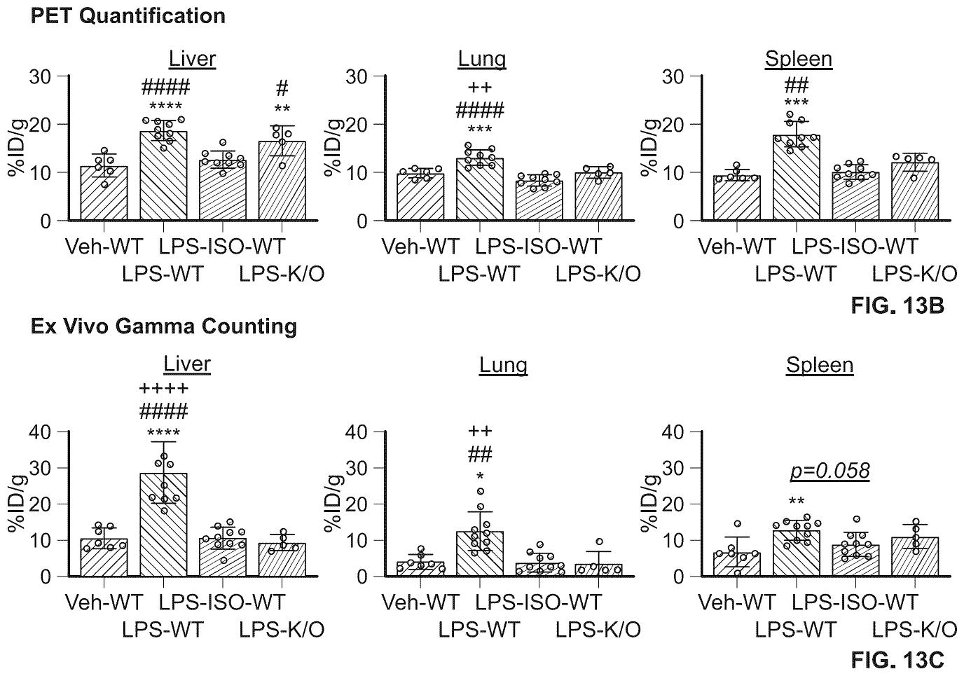

Pilot TREM1-PET imaging of LPS-WT versus Veh-WT mice revealed no significant difference in signal in the liver or spleen at 3 hpi. Conversely, quantification of images at 20 and 40 hpi demonstrated significantly higher signal in both liver and spleen of LPS-WT mice, without any substantial difference in signal-to-noise between time points. RT-qPCR data revealed higher levels of TREM1 in the liver, lungs, and spleen of LPS-WT compared to Veh-WT mice at 3-24 hours, with no significant difference at 72 hours. Hence, 20 hpi of ⁶⁴Cu-TREM1-mAb (˜24 hours pi LPS) was chosen as the optimal timepoint to perform imaging for all subsequent studies (See FIG. 13A).

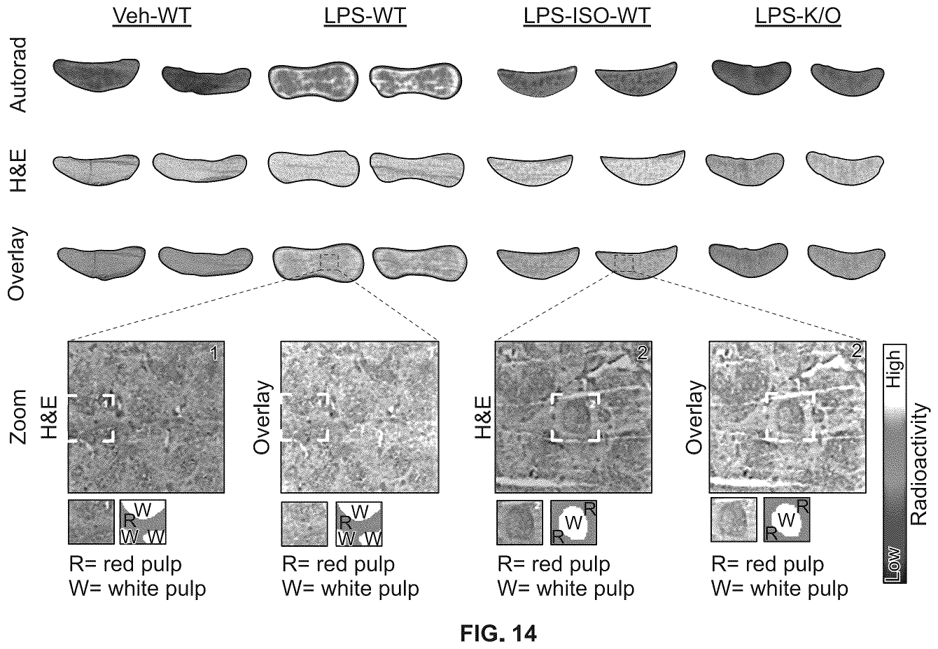

Quantification of PET signal in peripheral tissues from a larger follow-up study revealed significantly elevated TREM1-PET signal in liver (p<0.0001), lungs (p=0.0005), and spleen (p=0.0003) of LPS-WT compared to Veh-WT mice (See FIG. 13B). Importantly, LPS-WT mice imaged with ⁶⁴Cu-Isotype-control-mAb exhibited significantly reduced signal compared to LPS-WT mice imaged with ⁶⁴Cu-TREM1-mAb (liver: p<0.0001, lungs: p<0.0001, spleen: p=0.0011), confirming the specificity of ⁶⁴Cu-TREM1-mAb. Specificity was further illustrated by significantly lower PET signal in the lungs of LPS-K/O mice (vs LPS-WT: p=0.0016) and the fact there was no significant difference in splenic signal between Veh-WT and LPS-K/O mice. In vivo findings were validated by ex vivo gamma counting of liver, lung, and splenic tissues (FIG. 13C) and high-resolution autoradiography of the spleen overlaid with H&E staining (FIG. 14). Autoradiography revealed a distinct pattern of ⁶⁴Cu-TREM1-mAb binding restricted to the marginal zone and red pulp (FIG. 14), which contain macrophages; uptake was not observed in the T- and B-cell-rich white pulp.

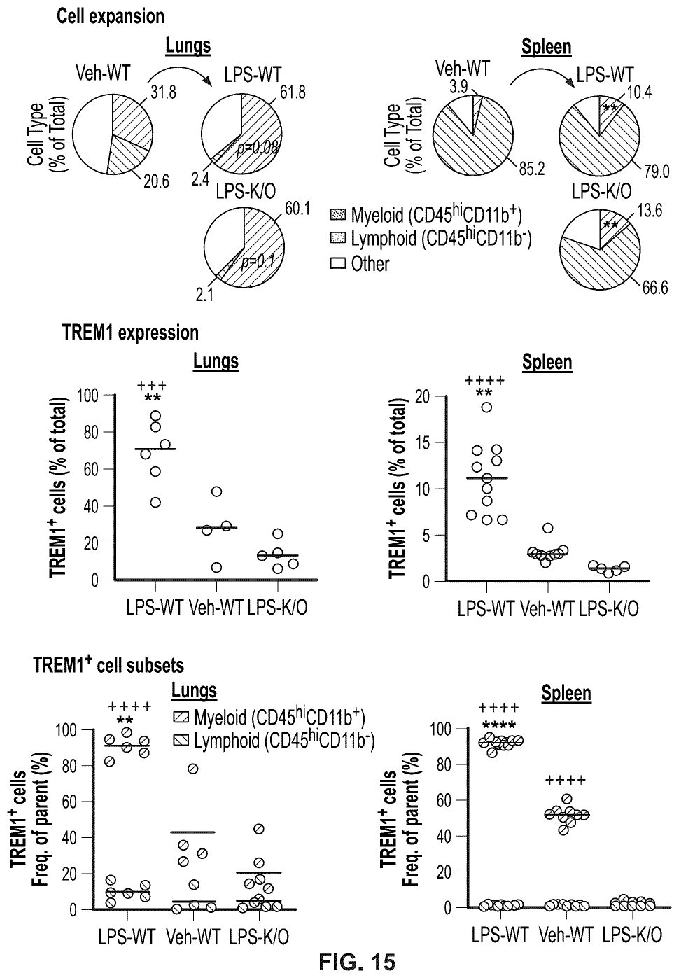

TREM1-PET imaging was further corroborated by flow cytometry data, which showed significant increases in the frequency of the CD45^{hi}CD11b⁺ myeloid cells in the spleens of LPS-WT mice (versus Veh-WT: p=0.0033, and LPS-K/O mice: p=0.0018) with an upward trend observed in the lungs (Veh-WT:p=0.08, LPS-K/O: p=0.01, FIG. 15). Notably, significant increases in TREM1⁺ cell frequency were observed in the lungs and spleen of LPS-WTs compared to Veh-WT (lungs: p=0.0016; spleen: p=0.0075) and LPS-K/O (lungs; p=0.0001; spleen: p<0.0001) mice, with expression highly restricted to myeloid populations (FIG. 15). Subsequent characterization of myeloid cells revealed constitutive TREM1 expression on splenic CD45^{hi}CD11b⁺Ly6G⁺ neutrophils in Veh-WT and LPS-WT compared to LPS-K/O mice (Veh-WT: p<0.0001; LPS-WT: p<0.0001), with more significant expression detected in LPS-WT versus Veh-WT mice (p=0.0002). Similar results were observed in the lungs (vs LPS-K/O: LPS-WT: p=0.0114; Veh-WT: p=0.059). Substantial TREM1 upregulation was also demonstrated on CD45^{hi}CD11b⁺Ly6G⁻ monocyte/macrophages/dendritic cells (DCs) in LPS-WT mice (vs Veh-WT: lungs: p=0.0022, spleen: p<0.0001; vs LPS-K/O: lungs: p=0.0002, spleen: p<0.0001).

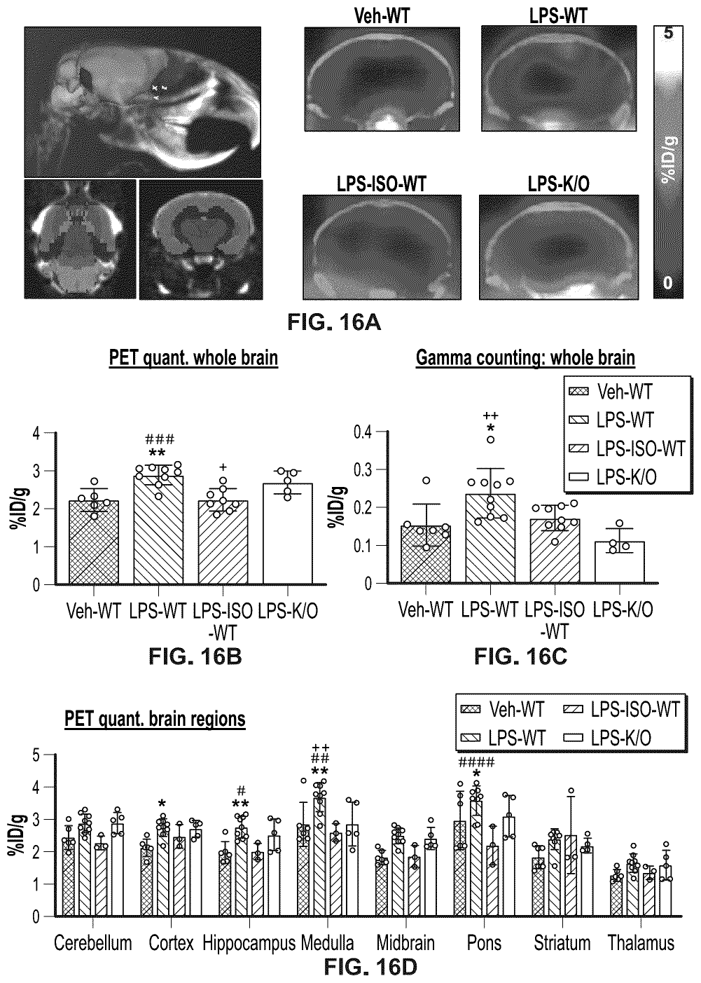

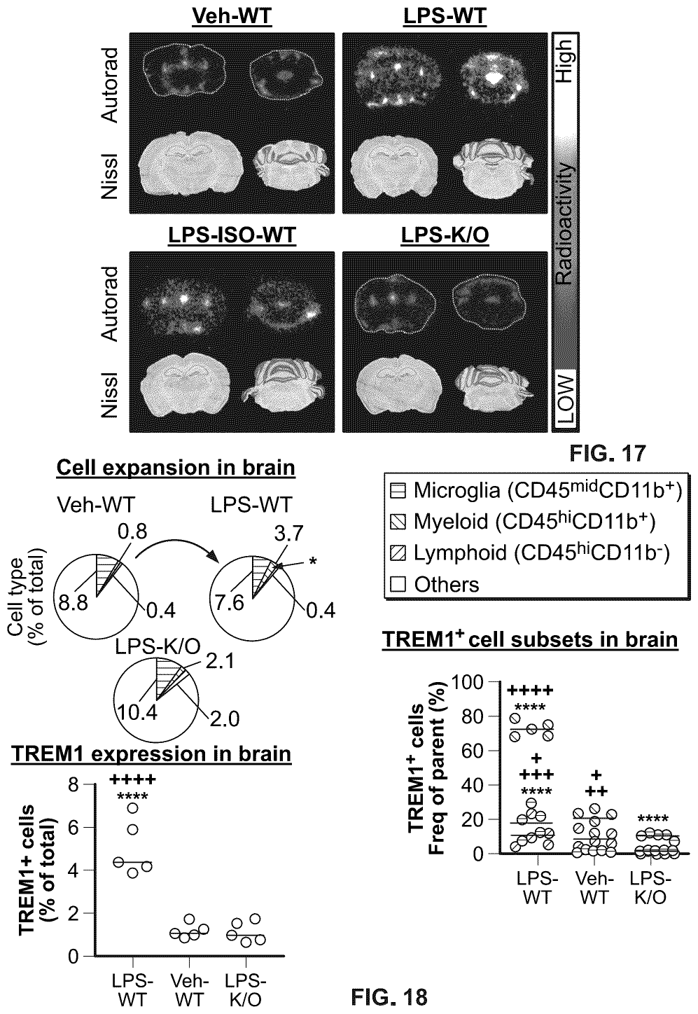

Quantification of brain PET/CT images revealed significantly higher binding of ⁶⁴Cu-TREM1-mAb in the whole brain of LPS-WT compared to both Veh-WT (p=0.0012) and LPS-ISO-WT (p=0.0005) mice (FIG. 16A-B). This is supported by significantly reduced signal in whole brain tissues of LPS-K/O vs LPS-WT animals, as assessed by gamma counting following perfusion to remove unbound intravascular tracer (p=0.0037) (FIG. 16C). Regional quantification demonstrated significantly increased ⁶⁴Cu-TREM1-mAb signal in the cortex (p=0.0313), hippocampus (p=0.0035), medulla (p=0.0017), midbrain (p=0.0169), and pons (p=0.0337) of LPS-WT versus Veh-WT mice (FIG. 16D). Signal also increased compared to LPS-ISO-WT in the hippocampus (p=0.0428), medulla (p=0.0011), and pons (p<0.0001). Reduced signal in LPS-K/O compared to LPS-WT mice was only observed in the medulla (p=0.0037) and not in the whole brain or other regions. High resolution ex vivo autoradiography of coronal brain sections demonstrated specific binding of ⁶⁴Cu-TREM1-mAb in the cerebellum, cortex, hippocampus, and medulla of LPS-WT mice (FIG. 17). Moreover, flow cytometry and RT-qPCR further support these findings. Flow cytometry demonstrated a significant increase in CD45^{hi}CD11b⁺ myeloid cell infiltration into the brains of LPS-WT versus Veh-WT animals (p=0.0112). Conversely, resident CD45^{mid} CD11b⁺ microglia and CD45-CD11b⁻GFAP⁺ astrocyte populations did not demonstrate significant changes in frequency post-LPS treatment (p=0.8098 and p=0.0654 respectively) (FIG. 18). As in the periphery, increased TREM1 cells were observed in the brains of LPS-WT animals (vs Veh-WT: p<0.0001; vs LPS-K/O: p<0.0001), with TREM1 predominantly expressed on myeloid cells (FIG. 18). Brain TREM1 upregulation in LPS-WT mice was observed on both CD45^{hi}CD11b⁺Ly6G⁺ neutrophil (vs Veh-WT: p=0.0004; vs LPS-K/O: p<0.0001) and CD45^{hi}CD11b⁺Ly6G monocyte/macrophage/DC (vs Veh-WT: p<0.0001; vs LPS-K/O: p<0.0001) populations. Resident brain CD45^{mid}CD11b⁺ microglia showed a significant, yet comparatively low, increase in TREM1 (vs Veh-WT: p=0.0005; vs LPS-K/O: p<0.0005) (FIG. 18), as previously demonstrated after ischemia. Low levels of TREM1 were also detected on CD45^{hi}CD11b⁻ lymphoid cells in WT mice compared to K/O mice (LPS-WT: p=0.0162; Veh-WT: p=0.0306). TREM1 was not significantly expressed on astrocytes. Increased TREM1 mRNA expression in the brains of LPS-WT compared to Veh-WT mice, at 3-24 hours after LPS challenge, reinforced these findings.

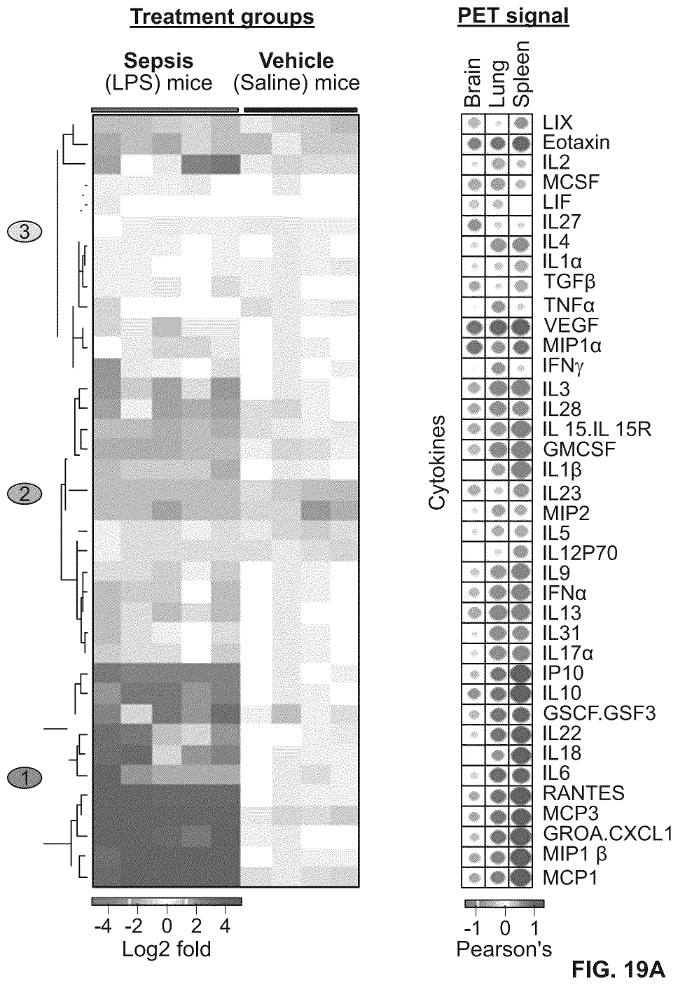

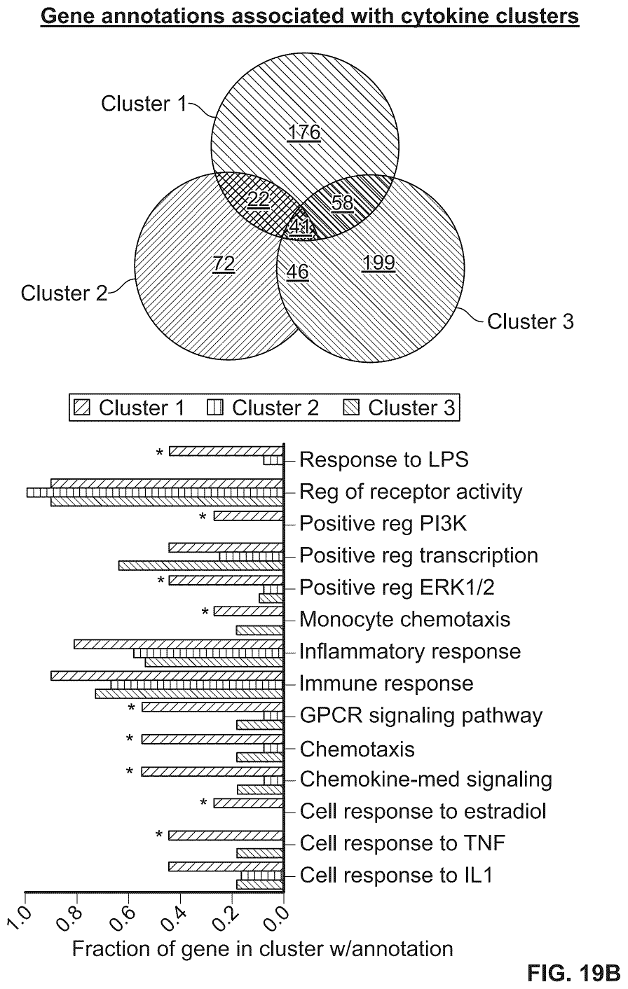

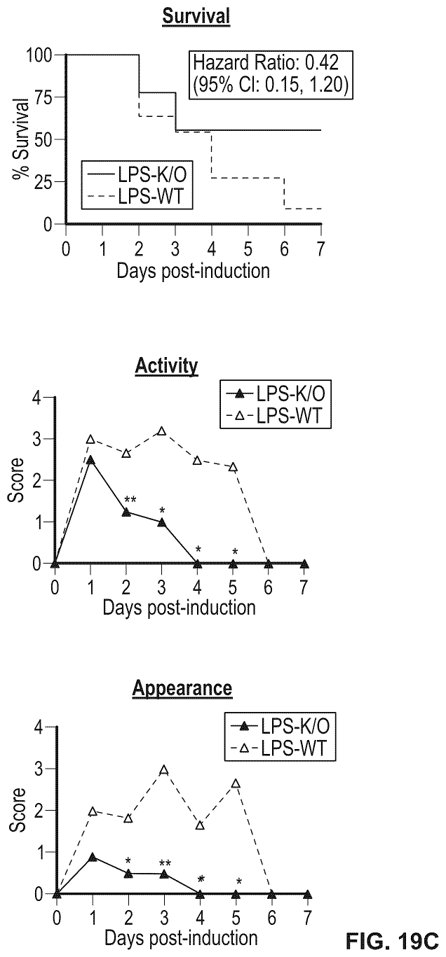

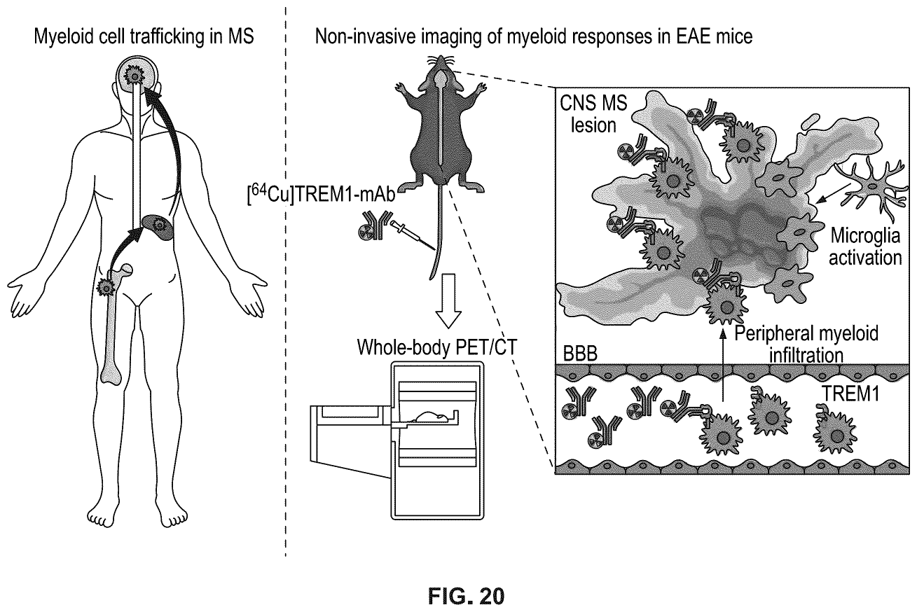

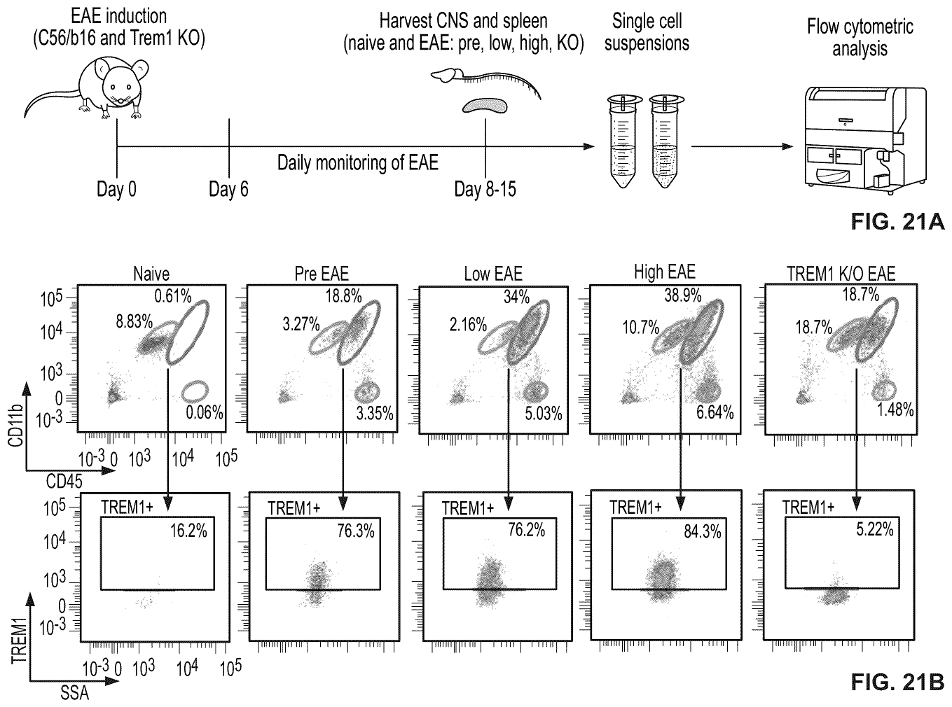

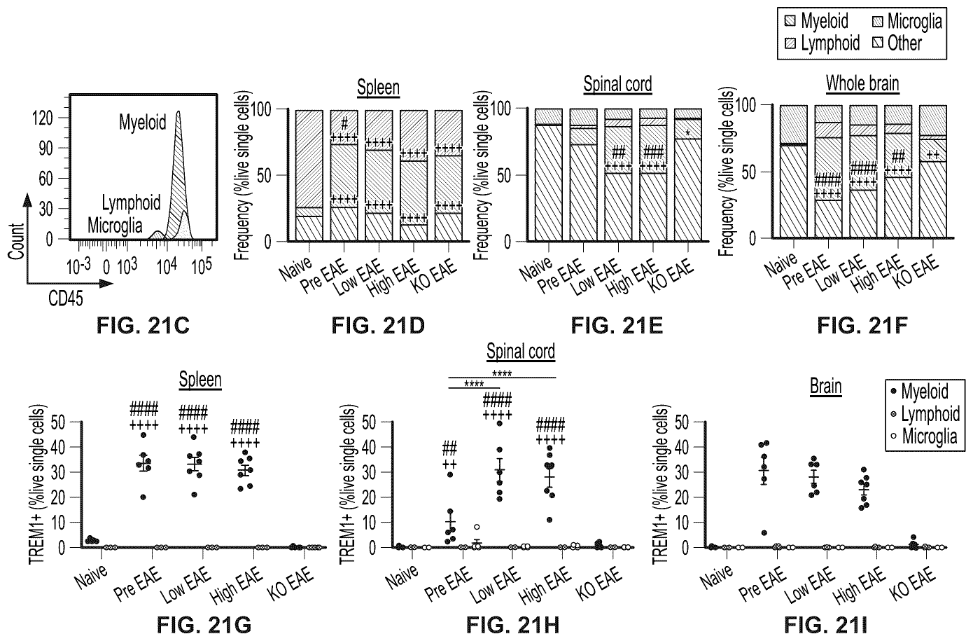

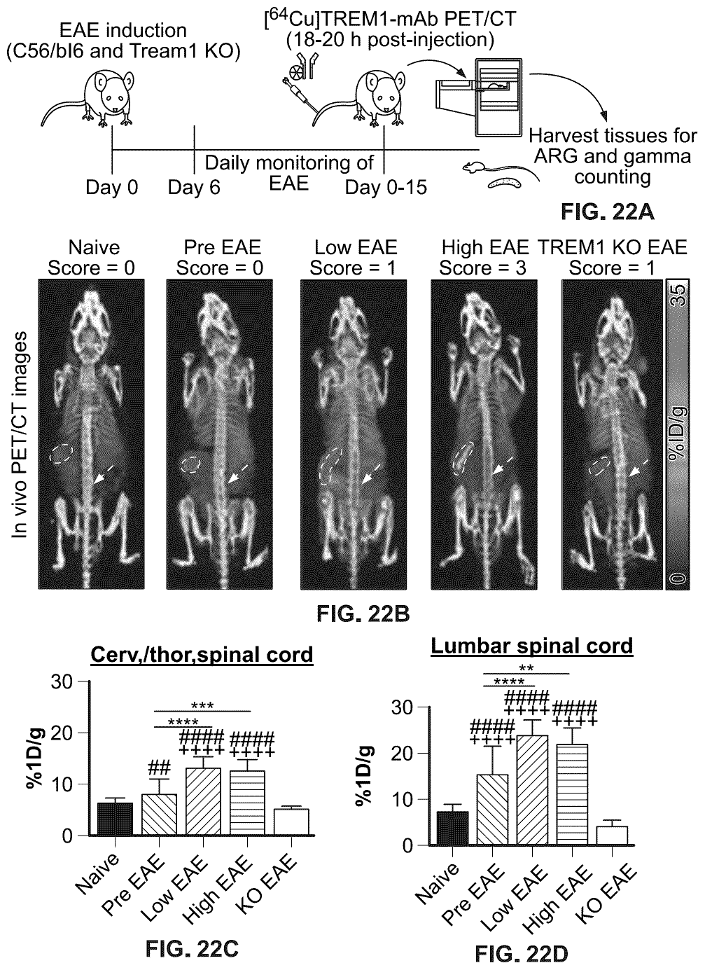

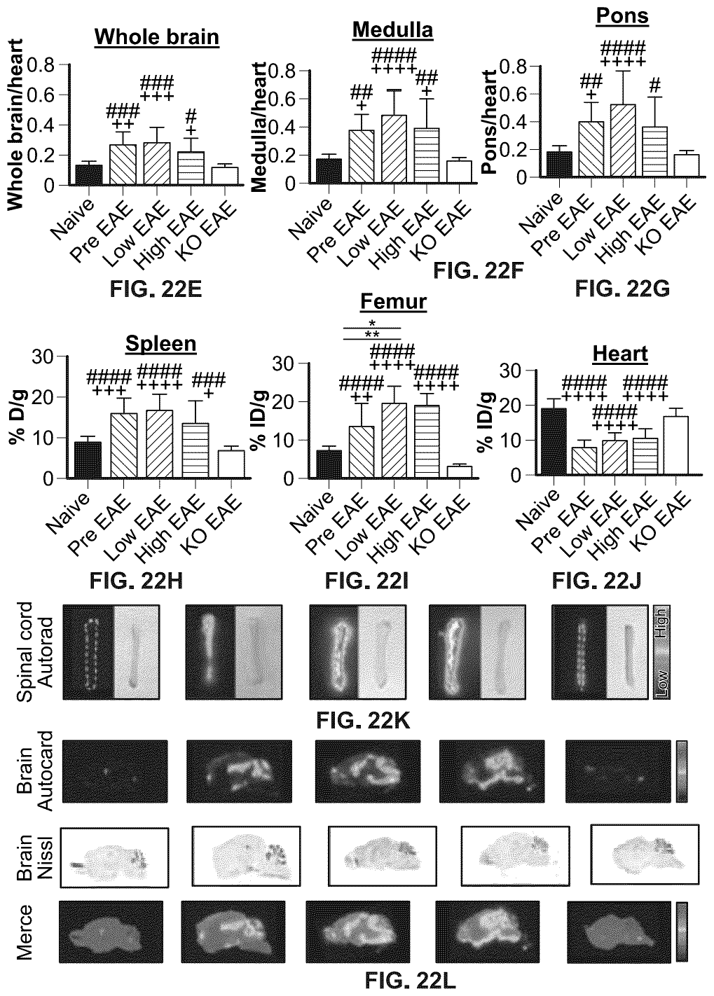

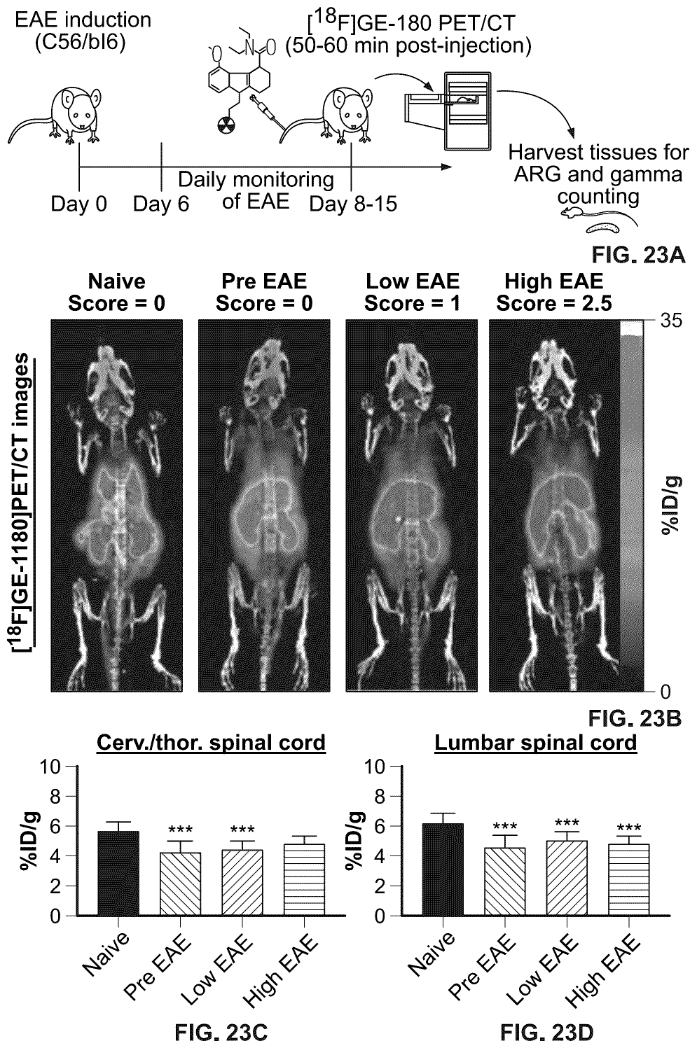

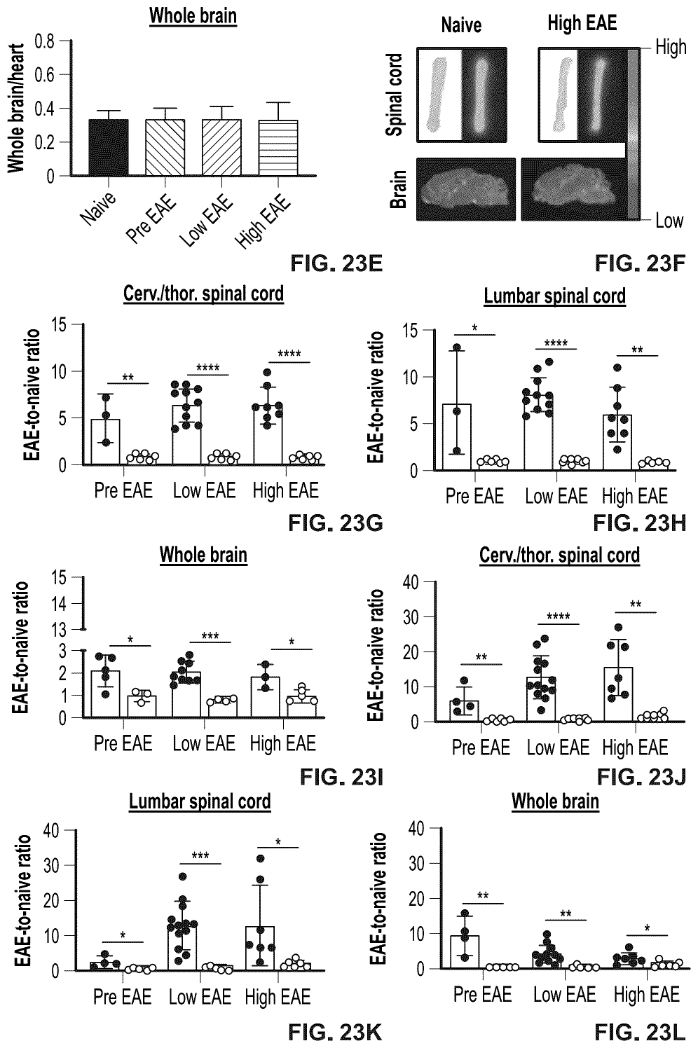

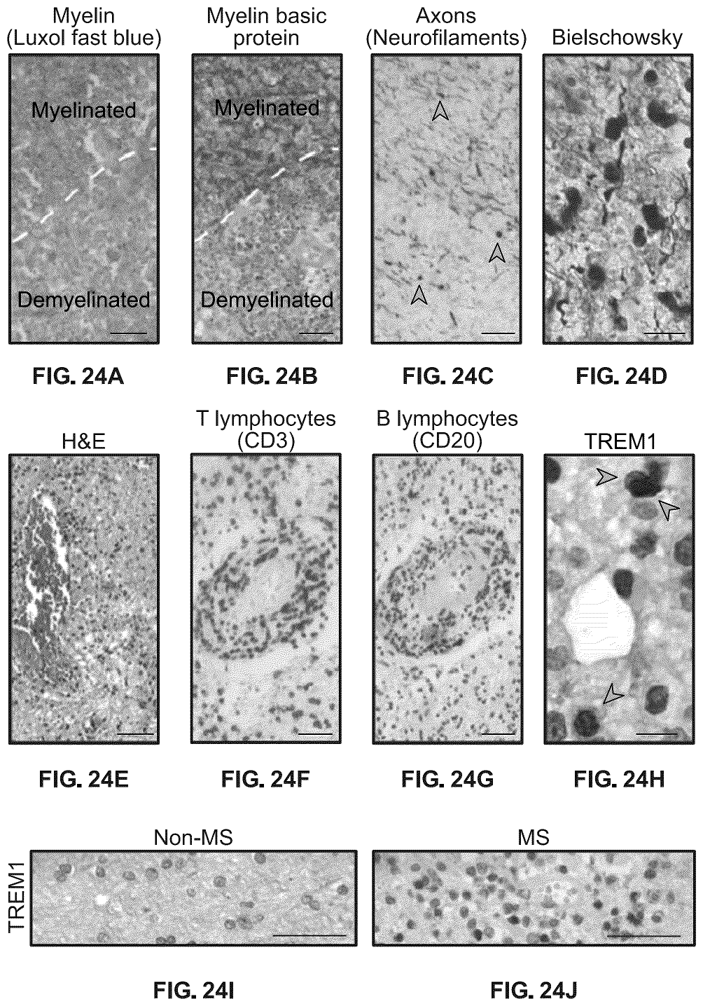

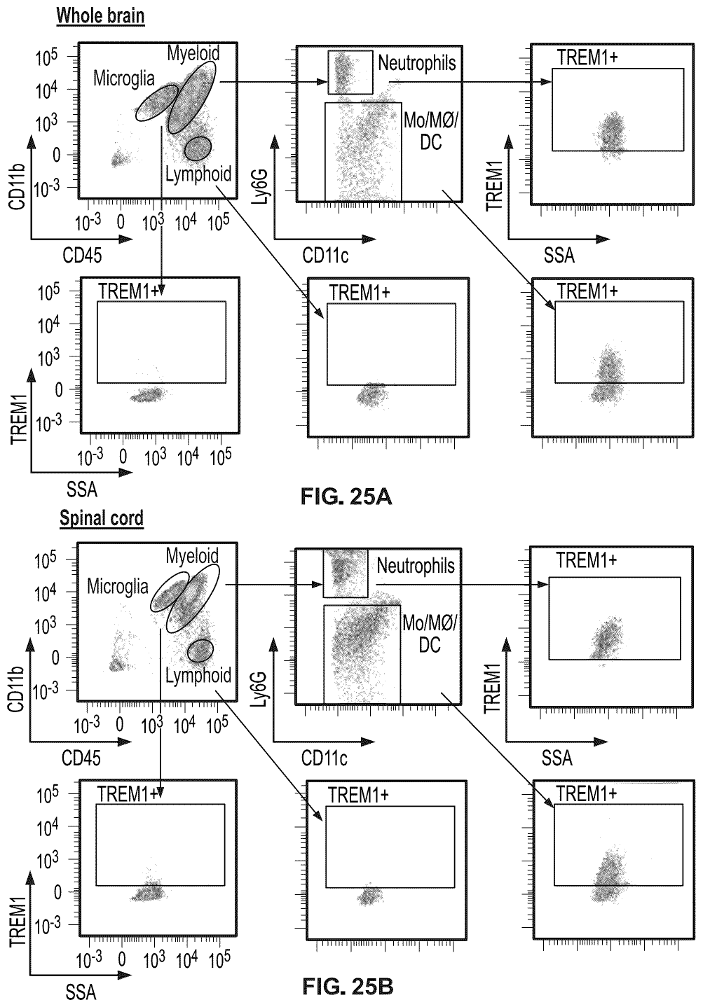

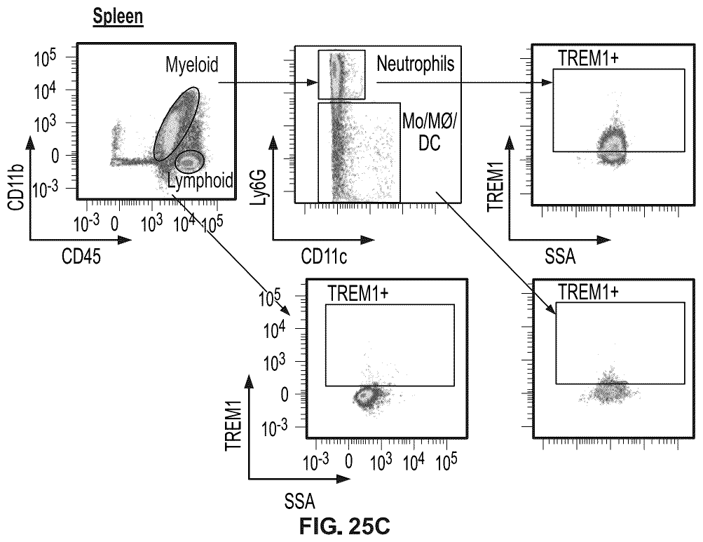

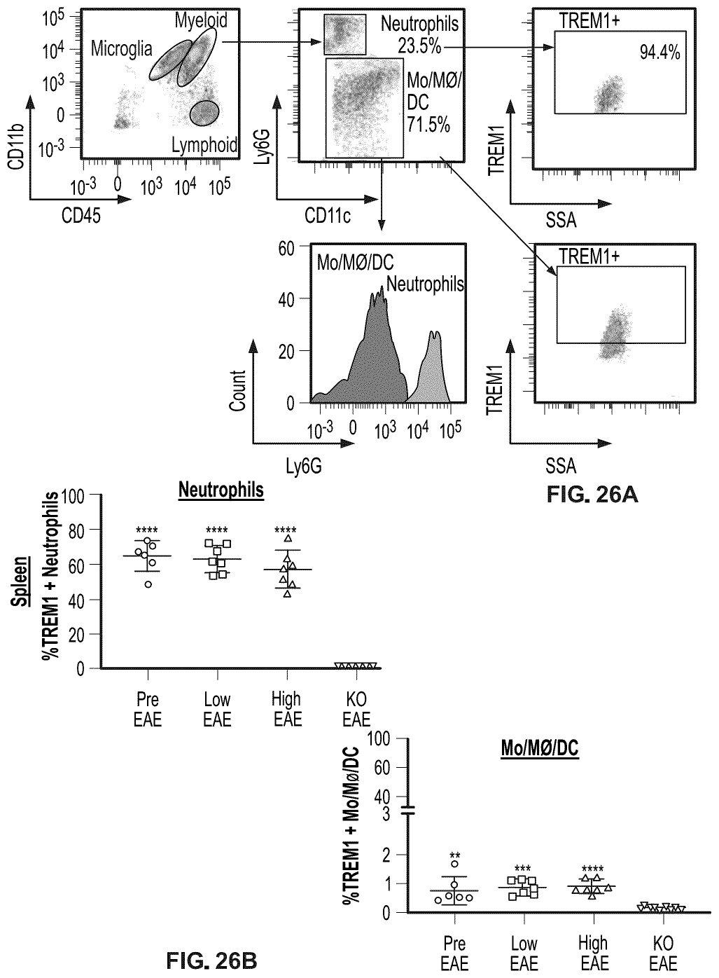

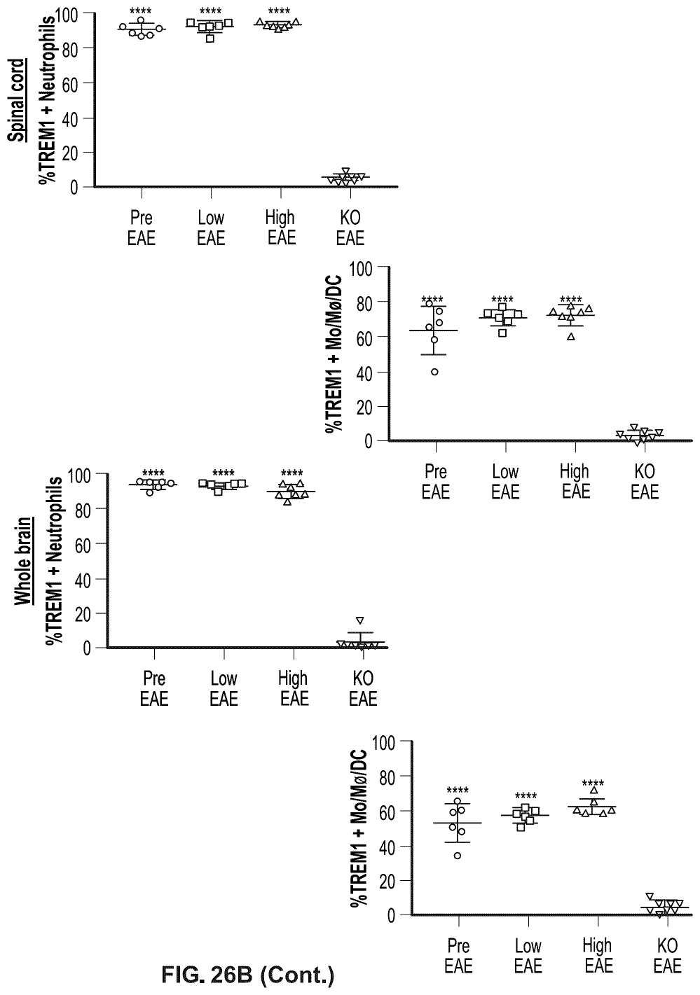

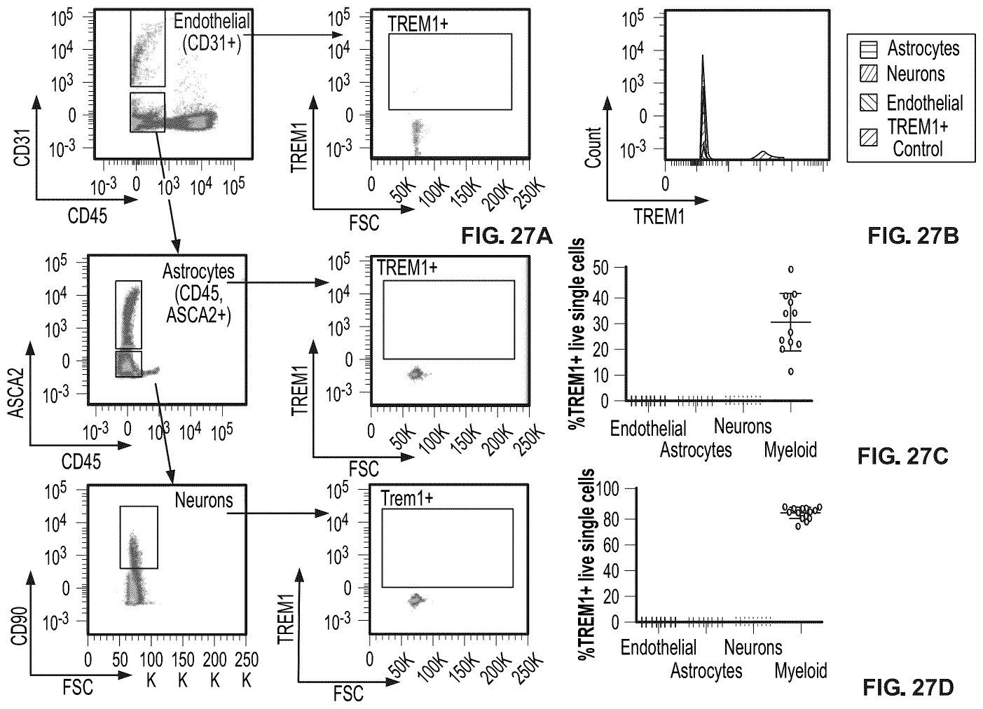

To further assess the relationship between TREM1-PET and peripheral inflammation, unsupervised hierarchical clustering of plasma cytokine signatures was performed (FIG. 19A). Three primary cytokine clusters were revealed in LPS-WT compared to Veh-WT mice: cluster 1 was upregulated, cluster 2 downregulated, and cluster 3 exhibited no changes. Notably, TREM1-PET signal in the spleen exhibited strong positive correlations with cluster 1 cytokines (FIG. 19A). Similar, but weaker, trends were observed for TREM1-PET signal in lungs, while TREM1-PET signal in the brain exhibited a significant correlation with only VEGF and MIP1α cytokines in cluster 1. Automatic functional gene annotations indicated distinct biological roles for these clusters (FIG. 19B). The majority fraction of all cytokines in the three clusters were annotated with the terms inflammatory response and immune response (FIG. 19B). Over-representation analysis identified nine unique annotations as significantly over-represented from cluster 1, notably “response to LPS,” “monocyte chemotaxis,” and “chemokine-mediated signaling,” suggesting association with pro-inflammatory responses to LPS. Cell response to TNF was significantly enriched in cluster 1. Although cell response to IL1 was annotated in a similarly high fraction of cluster 1 genes, it did not reach significance.

Given the link between TREM1 expression levels, TREM1-PET signal, and pro-inflammatory cytokine signatures in response to LPS-induced sepsis, whether excessive inflammation associated with TREM1 expression was linked to sickness behavior was evaluated. Genetic knockout of TREM1 led to statistically-improved motor activity (p=0.0281-0.0031) and appearance (p=0.0424-0.0043) of LPS-K/O compared to LPS-WT mice (FIG. 19C). Additionally, data from a small survival study (n=10) indicate improved survival in LPS-K/O mice by day 7 (55% vs 10%, p=0.1151). Investigation in larger cohorts is needed to further assess these promising results (FIG. 19C).

### Example 7: TREMl is a Highly Specific Biomarker of Peripheral Myeloid Cells in EAE

To evaluate TREMl as a biomarker of maladaptive myeloid-driven immune responses in the EAE mouse model of MS, single cell flow cytometry was performed on spleen. spinal cord. and brain tissues from wildtype (WT) naive, WT EAE, and Treml KO EAE mice. EAE disease severity was assessed using a standardized scoring method (Hooke Laboratories: https://hookelabs.com/services/cro/eae/MouseEAEscoring.html). Tissues were harvested from WT EAE mice at pre-symptomatic (pre: score 0, no paresis/paralysis,˜1 g weight loss in 24-48 b), low EAE (score 0.5-2, bind limb paresis/paralysis), and high EAE (score 2.5-4.5, hindlimb paralysis and forelimb paresis) disease states to profile the temporal dynamics of myeloid cells across disease progression. First, we assessed the proportions of microglia (CD45in1CD 1 I b+). peripheral myeloid (CD45hiCD 11 b+). and lymphoid (CD45+c o 11 b−) cells across all groups to confirm CNS infiltration of peripheral immune cells during EAE using flow cytometry. Significant myeloid cell expansion was confirmed in the spleens of all EAE groups (pre, low. high, and KO EAE) compared to naive mice (P:S 0.0001). CNS-infiltration of peripheral myeloid cells was dramatically increased in the spinal cord of low and high scoring EAE mice compared to both naive and KO EAE mice (P:S 0.0001). with a trend towards significance observed in pre EAE mice (P=0.0683 vs naive). Increased myeloid cell frequency was also seen in KO EAE compared to naive mice (P=0.0113). A similar pattern of myeloid cell infiltration was observed in the brains of EAE-induced mice (P:S 0.0001-0.0022). A significant reduction in microglia frequency was found in the brains of all WT EAE (pre, low, high) mice versus naive (P 0.0085-0.0296). This was not observed in KO EAE mice versus naive, suggesting that this effect is likely due to the magnitude of peripheral myeloid cell infiltration in WT EAE mice Next. we investigated TREMl expression on immune cell populations. TREMi was selectively expressed on peripheral myeloid cells in WT EAE mice with no expression evident on microglial or lymphoid cells). Elevated TREMi+O myeloid cells were observed in the spleen, spinal cord, and brains of WT EAE compared to naive and KO EAE animals (P:S 0.0016-0.0001). Notably. CNS-infiltration of TREMl+ myeloid cells was evident in the spinal cord and brains of WT pre EAE mice prior to clinical onset. Spinal cord TREMl+ myeloid cell infiltration increased further upon symptom development during low and high disease (P:S 0.0001). In contrast, brain TREMJ+ myeloid cell infiltration reduced with disease progression, displaying lower TREMl+ myeloid cell levels in high scoring disease compared to pre EAE states (P:S 0.0001). To better characterize the subtypes of cells expressing TREMi. myeloid cells populations from EAE mice were further segregated into neutrophils (CD45hiCD 11 b+Ly6G+) and monocyte (Mo)/macrophage (MO)/dendritic cells (DC) (CD45hiCDI Ib4 Ly6G−) populations. The percentage of TREM 1+neutrophils and TREM 1+Mo/MO/DCs was markedly increased in the spleen, spinal cord, and brain of WT EAE compared to KO EAE mice (P:S 0.01-0.0001). Notably. a high proportion of neutrophils (spinal cord: 92-94.7%. brain: 93.1-96.7%) and Mo/MO/DCs (spinal cord: 64.8-72.8%. brain: 56.8-64.3%) that infiltrated the CNS in WT EAE mice exhibited TREMl expression. To further confirm the cell specificity of TREM 1, we performed additional flow cytometry studies of spinal cord tissue to isolate endothelial cells (CD31+). neurons (CD4S-CD90+), and astrocytes (CD45-ACSA2+). Flow cytometric analysis determined that TREMi was not expressed on any of these cell types in EAE. Taken together, these data reveal TREMl as highly specific marker of peripheral myeloid cells, as well as an early and sustained biomarker of disease in EAE.

In vivo TREMl-PET enables early detection of disease in EAE mice prior to symptom onset. We subsequently assessed the ability of TREMl-PET imaging to detect and track peripheral myeloid cells in vivo. [⁶⁴Cu]TREMl-mAb PET/CT (10 min static) was performed in WT EAE, KO EAE. and naive mice 20 h post-injection. TREMi-PET images revealed markedly elevated signal in the spinal cord (white arrow), spleen (white outline), and bone of WT EAE mice compared to naive and KO EAE mice. [⁶⁴Cu]TREMl-mAb PET signal in naive and KO EAE mice was primarily observed in the heart and descending aorta, typical of antibody-based PET tracers that are residing in the blood pool and not binding to a specific target. Therefore, the increased signal observed in WT EAE mice indicates specific binding of [⁶⁴Cu]TREM 1-mAb to TREM I+ cells. PET image quantification confirmed a significant increase in [⁶⁴Cu]TREMl-mAb binding in the lumbar (15.82-24.23% ID/g [injected dose per gram]) and cervical/thoracic (8.31-13.35% ID/g) spinal cord of WT EAE versus naive and KO EAE mice (P:S 0.01-0.0001). Notably, increased [⁶⁴Cu]TREM1-mAb binding was evident in pre EAE mice so and increased further upon symptom manifestation in low and high EAE mice This signal pattern reflects the elevated TREM 1+ myeloid cell infiltration characterized using flow cytometry, and further supports the specificity and selectivity of our TREMl probe. Brain PET signal was expressed as a ratio over the heart signal for individual mice to account for the contribution of unbound tracer in the blood pool. Increased PET signal ratios were detected in WT EAE compared to naive and KO EAE mice in the whole brain as well as the white matter rich regions. pons and medulla (PS 0.05-0.0001). CT-guided quantification of the spleen and femur signal demonstrated increased [⁶⁴Cu]TREMl-mAb binding in WT EAE (spleen: 13.84-16.98 3/4ID/g, femur 13.76-20.01%1O/g) compared to naive and KO EAE mice (P S 0.001-0.000 I), whereas reduced tracer accumulation in the heart was observed for all WT EAE mice (P::;;: 0.01 vs. naive and KO EAE). This is in line with increased [⁶⁴Cu]TREM1-mAb remaining in the blood pool of naive and KO EAE mice due to lower or lack of TREM 1+ cells respectively in these mice. To investigate whether the increased TREMl-PET signal in the CNS was a result of MOG35.5s-induced inflammation or due to the action of the immune adjuvant (complete Freund's adjuvant, CFA) used to induce EAE. control mice were induced with a CFA emulsion without MOG35-55. and PET imaging was performed. Peripheral immune responses in the bone marrow and spleen in control mice were observed with TREMl-PET. Significantly higher signal was detected when comparing all WT EAE groups to control mice in both the cervical/thoracic and lumbar spinal cord (PS 0.001). In contrast, no differences in binding were observed in the brain, suggesting increased TREM 1-PET brain signal is CF A-driven. Similarly. peripheral binding in the spleen and femur did not differ significantly between control and EAE mice, likely due to the peripheral immune responses initiated by CF A in these regions. In further support of this, principal component analysis (PCA) of TREMl-PET data identified the lumbar and cervical/thoracic spinal cord as the regions that most specifically distinguish EAE mice from naive. Furthermore, TREMl-PET signal in spinal cord regions was found to be a highly sensitive and specific approach to identify EAE disease as demonstrated by ROC (receiver operating characteristic) analysis. Following imaging. mice were perfused to remove the contribution of tracer in the blood pool, and high-resolution ex vivo autoradiography and gamma counting were performed. Autoradiography confirmed increased tracer retention in the brains and spinal cord of WT EAE mice with increased signal reaching up to 2- and 8-fold of that seen in naive mice respectively. Gamma counting of CNS and peripheral tissues further supported PET and autoradiography findings. Additionally, since free ⁶⁴Cu (if present due to tracer instability) will accumulate in the liver, liver signal was assessed and found to be similar across groups. This verified the in vivo stability of our [⁶⁴Cu]TREM1-mAb imaging approach in this model and highlights the strength of our observations.

TREMl-PET is a more sensitive tool for detecting neuroinflammation in EAE compared to TSPO-PET Next, we examined the sensitivity of TREMl-PET compared to the gold standard approach for assessing neuroinflammation in vivo for both preclinical and clinical research—TSPO-PET. Naive and EAE mice were injected with [¹⁸F]GE-180. a highly sensitive and selective tracer for TSPO in rodents (26), and PET images were acquired 50-60 min following tracer administration. In contrast to TREMl-PET, TSPO-PET did not detect the known increase in CNS myeloid cell infiltration in EAE mice. In fact. significantly reduced uptake was seen in cervical/thoracic and lumbar spinal cords of EAE compared to naive mice (P:S 0.01-0.001) This reduction in spinal cord signal is likely due to a combination of differences in tracer excretion between EAE and naive mice in addition to responses caused by CF A alone. and the lack of cell specificity of TSPO compared to TREMl. Specifically, peritoneum adhesions encompassing the spleen. mesentery, and stomach, as well as abdominal granulomatous inflammation, have been reported in mice injected with CFA (27). These Cf A-induced effects in the abdomen may explain the increased [¹⁸F]GE-180 abdominal signal observed in EAE mice. therefore reducing the availability of tracer to bind to spinal cord regions. This hypothesis is further supported by the decreased CNS [¹⁸F]GE-180 binding and increased abdominal signal in control mice who received CFA emulsion without MOG3s ss (P:S 0.01-0.001) compared to naive untouched mice. of note, no significant differences were observed in the brain of EAE versus naive mice. Ex vivo autoradiography supported these in vivo PET findings ( ). To directly assess the sensitivity of [⁶⁴Cu]TREMl-mAb and [¹⁸F]GE-180 for detecting myeloid cell inflammatory responses in the CNS. we compared EAE-to-naive ratios using ex vivo autoradiography and gamma counting. Significantly elevated [⁶⁴Cu]TREMl-mAb binding ratios were observed in the spinal cord and brains of EAE mice: reaching 14-17-fold than seen with [¹⁸F]GE-180 (P:S 0.05-0.0001). Additionally, quantitative PCR results confirmed TREMJ transcription was upregulated in WT EAE spinal cord tissue compared to naive animals at a significantly higher fold than TSPO (cervical/thoracic: 25.4 vs 585.4-fold. lumbar: 18.3 vs 1027.5-fold, P:S 0.0017-0.0001). These results corroborated the high sensitivity of TREMl-PET for detecting pro-inflammatory myeloid cells and demonstrate the ability of TREMl-PET to successfully monitor active EAE with increased sensitivity and selectivity compared to TSPO-PET.

TREMl-PET signal correlates with a pro-inflammatory cytokine/chemokine profile To better understand the immune signature associated with a positive TREMi-PET signal. we performed multi-plex analysis of plasma cytokine profiles in WT EAE and naive mice. Analysis of individual cytokines revealed increased levels of chemokines/cytokines associated with pro-inflammatory responses and disease development/proliferation in EAE groups (28, 29) Significant increases were observed in IP-10/CXCL0, GCSF/CSF3, MCP3, IFNγ, MIP IP, and TNFα in high (P=0.024-0.048) and low EAE (P=0.036) compared to naive mice. A trend towards significance was also seen for these cytokines/chemokines in pre EAE animals (P=0.05-0.11). Moreover, TREMi-PET signal in the spleen and CNS positively correlated with many of these cytokines/chemokines including TNFα, RANTES, MCP3, IP-10iCXCLI 0. IL-18, and IFNγ. However, correlation was identified with other cytokines/chemokines associated with EAE/MS including GMCSF, IL-6 and IL-23 to name a few. These results indicate that blood tests alone cannot successfully identify EAE disease. Nonetheless, adjunct assessment of blood cytokine levels with TREMl-PET imaging, which we have shown is a more sensitive indicator of EAE disease. revealed a positive correlation between TREMl signal and pro-inflammatory chemokines/cytokines.

TREMJ gene expression is associated with a pro-inflammatory neuroimmune response To examine the immune responses associated with TREMJ expression, neuroinflammatory gene signatures from spinal cord tissues of naive and EAE mice were assessed via the Nanostring preset neuroinflammation panel. Significantly distinct immune signatures in EAE and healthy naive mice were revealed with PCA. Heat map hierarchical clustering reinforced these findings. By deploying the Nanostring gene set enrichment designations, we determined that neutrophil degranulation and regulation of the complement cascade were among the most upregulated during EAE pathology, whereas neuronal system and mitophagy gene sets were downregulated. To further characterize neuroimmune changes represented by TREMl expression, Treml gene expression was correlated with canonical homeostatic or inflammatory markers. Treml showed strong positive correlation with pro-inflammatory innate immune targets (i.e., Clec7a, Jl-I a,II-1/3, Tnf, Anxal, Len 2, Cc/2, Cc/5, Cxc/10, Ccr2, Cc/7 (28-33))(P=0.0046-8.6e-6). Trem 1 was also negatively correlated with homeostatic. neuronal, or myelination markers (i.e., Arc, Opalin, Entpd2, Gjal, Rbfox3 (30, 34-37)) (9e-4<P<0.024).

Blocking TREMi signaling genetically and pharmacologically attenuates EAE severity. To explore the biological relevance of TREMl in EAE development and progression, we assessed disease trajectory in KO EAE compared to WT EAE animals. While 83% of WT mice developed EAE, only 40% of Trem/KO mice developed symptoms during the same timeframe. Treml KO also reached significantly lower levels of disease severity (P˜0.01-0.001). Next, to determine if blocking TREM I signaling during disease development could have therapeutic effects, we treated WT EAE animals with LP 17 (10 or 15 mg/kg)—a 17 mer peptide decoy receptor, previously shown to attenuate TREMi signaling (25, 38)—and compared disease progression to vehicle (saline) treated mice. Treatment was initiated at the pre EAE stage as this is the earliest time-point disease could be detected, and because our data showed TREMl to be highly expressed at this stage. Significant attenuation of disease severity was seen in WT EAE animals treated with 15 mg/kg LPI 7, but not 10 mg/kg LPI 7, compared to saline treated mice (P:S 0.05-0.01). These results suggest that TREMl signaling contributes to disease progression and is a potential therapeutic target to reduce symptom severity.

TREMl is a clinically translatable biomarker for MS To establish the clinical relevance of TREMl in MS. we probed the presence of TREMl+ cells in human MS brain lesions. We obtained a temporal lobe white matter brain biopsy from a treatment-naive adolescent female subsequently diagnosed with tumefactive MS—a rare form of MS characterized by demyelinating lesions greater than 2 cm. Histopathological processing revealed a significant loss of myelin as reflected by both Luxol Fast Blue histochemical preparation and myelin basic protein staining. Immunostaining for neurofilaments and Bielschowsky's silver stain further demonstrated severe axonal degeneration and formation of axonal bulbs. Additional staining with Hematoxylin-Eosin (H & E) revealed significant perivascular infiltration of mononuclear inflammatory cells. as well as infiltration of CD3+ T cells and CD20+ B cells, although the latter to a lesser extent. Adjacently stained biopsy sections also demonstrated high numbers of TREM 1+ cells, compared to control non-MS white matter, which was nearly devoid of TREMl+ cells.

Discussion

Activated myeloid cells are an early and persistent feature of MS and play a central role in disease progression and remission. However, due to a lack of non-invasive myeloid-specific imaging techniques, our current understanding of the in vivo temporal dynamics, spatial distribution, and beneficial versus toxic nature of innate immune responses in clinical MS is severely limited. Thus, clinical imaging strategies that enable visualization of innate immune status in the CNS are essential to improve disease staging and therapeutic monitoring in MS patients Motivated by the clinical need for a functionally relevant myeloid-specific 1magrng biomarker. we sought to validate TREM I as a biomarker of proinflammatory myeloid cells in the EAE mouse model of MS. We first confirmed the infiltration and expansion of myeloid cells with EAE induction and subsequently examined which immune cell subtypes expressed TREMi in CNS and spleen tissues. Flow cytometry revealed highly selective expression of TREMl on peripheral myeloid cells (i.e., neutrophils, monocytes. macrophages, and dendritic cells). Importantly. no expression was observed on lymphoid cells. endothelial cells, microglia, astrocytes, or neurons within spinal cord tissue. In the CNS. TREMi-myeloid cell infiltration was found to be dramatically increased in WT EAE mice (vs naive and Treml KO EAE) as early as the pre-symptomatic disease state. Together with the extensive data on rodent and human models that support TREMl as a potent amplifier of proinflammatory innate immune responses (J 9-24, 39). these data reveal TREMi as an early and specific biomarker of toxic peripheral myeloid cell responses in EAE. Owing to the high specificity of TREM 1 expression on peripheral myeloid lineage cells, we investigated the ability of TREMi-PET to track whole-body maladaptive innate immune responses in vivo using our novel [⁶⁴Cu]TREMl-mAb tracer. We previously demonstrated the high specificity of [⁶⁴Cu]TREM1-mAb in vitro. with 24-fold higher tracer binding seen in TREMl-transfected versus untransfected HEK293 cells (25). Here, in vivo TREMl-PET accurately detected disease in WT EAE mice at a very early stage of disease (prior to any muscle weakness/paralysis) in addition to mice with increasing disease severity. Markedly elevated [⁶⁴Cu]TREM 1-mAb signal was identified in the spinal cord, brain, spleen. and bone of WT EAE compared to both negligible levels in naive and Trem I KO EAE mice. Notably, the absence of in vivo signal in KO EAE mice provides further evidence of the specificity of this tracer. Ex vivo biodistribution and high-resolution autoradiography of tissues following perfusion—to remove unbound intra vascular tracer—confirmed in vivo findings and further reinforced the specificity of [⁶⁴Cu]TREM 1-mAb. TSPO-PET is currently considered the current gold standard approach to detect neuroinflammation in vivo. In clinical research studies. TSPO-PET has shown variable capacity to detect microglial activation in MS patients. Some studies reveal increased signal at acute stage disease, while others show no differences (40-42). The inconsistent findings may be attributed to the lack of cellular specificity or functional information available for TSPO as a neuroimmune imaging biomarker. In this study, we demonstrate that TREMl-PET has superior sensitivity for detecting maladaptive innate immune responses in EAE mice than TSPO-PET. In contrast to TREMl-PET, in vivo TSPO-PET did not identify increased CNS inflammation known to occur in this model. Ex vivo EAE-to-naive ratios (using autoradiography and gamma counting) of CNS tissues following perfusion confirmed the markedly enhanced discrimination of EAE disease with [⁶⁴Cu]TREMl-mAb compared to [¹⁸F]GE-180. A major advantage of TREMl as a biomarker is its functional relevance. TREMi has been shown to specifically amplify pro-inflammatory innate immune responses overcoming the limitations of other imaging biomarkers of neuroinflammation such as TSPO. Here, we further characterize the proinflammatory cytokine milieu in EAE animals. which correlates with disease severity and notably TREM I-PET signal. Furthermore, we demonstrate that Trem/gene expression in EAE mice positively correlates with genes well characterized in proinflammatory signaling like Tnf a, Il-I/J, and Cxcll O and negatively correlates with homeostatic markers of neuronal integrity (Rbfox3) and myelination (Opalin). Ablation of Treml and attenuation of TREMl-signaling in models of peripheral inflammation have previously been shown to significantly reduce immune-driven injury and improve outcome measures (21, 25). Here, we confirm the strong link of TREM I to pathologic immune responses in the EAE mouse model. Genetic knockout of Treml resulted in significantly delayed EAE onset. Additionally. treatment with a pharmacologic agent known to attenuate TREM 1 signaling (LP 17) significantly reduces EAE disease severity. These results demonstrate the critical role TREMi plays in driving EAE and supports TREM 1 as a potential therapeutic target to modulate maladaptive innate immune responses. Current standard of care imaging techniques used to diagnose and monitor clinical MS (i.e., MRI) cannot provide sufficiently early or granular molecular information regarding patients' immune signatures in the CNS. The lack of non-invasive methods to assess CNS immune status in MS patients limits the ability to select the most appropriate therapy and obtain real time predictors of treatment response for a given patient. This is particularly pertinent considering the heterogenous treatment responses among patients with MS. Our finding that TREMJ+t immune cells are present in the brain lesions of a treatment naive MS patient supports TREMl as a clinically translatable biomarker and further investigation in clinical MS is warranted. Therefore: considering our evidence supporting TREMl as a specific, early, and functionally relevant biomarker of maladaptive innate immune responses. TREM I-PET imaging has high potential for significant clinical impact on early-stage diagnosis and therapy selection/monitoring. Specifically. the early upregulation of TREMi at a pre-symptomatic state indicates that PET imaging of TREMi in MS patients could potentially predict relapse early, prior to symptom worsening: and offer a novel therapeutic and diagnostic target for disease management. This study is not without limitations. Notably, the monoclonal antibody used to generate [⁶⁴Cu]TREM1-mAb cannot be directly translated for use in human subjects. However, we are in the process of radiolabeling an anti-human antibody, and anti-human antibody fragments are currently being generated for clinical use. To ensure effective translation of a humanized tracer. thorough preclinical evaluation must be performed, including in vitro uptake studies in human myeloid cell lines, human plasma stability assays and in vivo imaging in human Trem knock-in mice. It is also worth noting that preclinical investigation in animal models is an essential component to enable translation of diagnostic and therapeutic approaches, as well as to enhance our understanding of disease mechanisms. As the first myeloid-specific imaging approach, our rodent [⁶⁴Cu]TREMl-mAb tracer has the potential to transform our fundamental understanding of in vivo innate immune responses in a broad range of inflammatory conditions (including but not limited to atherosclerosis, arthritis, inflammatory bowel disease, numerous neurodegenerative diseases) and represents a tool for in vivo preclinical screening of novel immunomodulatory therapeutics. Here, we showed that specifically attenuating TREM 1 signaling via LP 17 treatment significantly reduced disease severity in EAE mice. However, TREMl-PET imaging in LPI 7-treated mice could not be performed since LP 17 acts as a decoy receptor for TREM I—binding available endogenous TREMl ligands and preventing the binding of our tracer. Finally it is worth noting that TREM 1 expression has previously been reported in pulmonary and aortic endothelial cells (43). thus endothelial TREMl expression should be validated in future organs/diseases of interest. In conclusion, TREM 1 is an early and highly specific biomarker of maladaptive innate immune responses. This is the first report of a highly specific PET imaging strategy for detecting pathogenic peripheral CNS-infiltrating myeloid cells in EAE. TREMi-PET accurately detects active EAE disease, even prior to symptom manifestation, and with increased sensitivity compared to the gold standard of TSPO-PET. TREMl signaling is associated with a pro-inflammatory immune signature, and dampening its function (e.g., via Treml KO or LPI 7 treatment) can alter disease progression. Moreover, TREM 1+ immune cells are evident not just in mice but in human early stage MS brain lesions—highlighting its promise as a clinically relevant imaging biomarker. Taken together, TREMl-PET imaging has high potential for significant clinical impact on early-stage diagnosis, therapy selection, and relapse monitoring for MS.

Materials and Methods

Experimental Design

Here we sought to investigate TREMi as a functionally relevant and translatable imaging biomarker of maladaptive innate immune responses using the EAE mouse model of MS. Flow cytometry of spinal cord. brain, and spleen tissue was performed to characterize TREM 1 expression on immune subsets of WT EAE mice across pre. low. and high EAE disease states (n=6-9/group). A separate cohort was used to assess the sensitivity and selectivity of TREMi-PET to detect peripheral infiltrating myeloid cells in different disease groups (WT naive, WT EAE, KO EAE, and WT control, n=9-12/group). Results were confirmed using ex vivo autoradiography and gamma counting of organs (n=4-13/group). Cardiac blood samples were taken from these mice prior to perfusion to assess cytokine profiles. To allow for accurate comparison of tracers, in vivo TSPO-PET using [¹⁸F]GE-I 80 (n=8-13/group) and subsequent ex vivo autoradiography (n=6-11/group) and gamma counting (n=3-5/group) was performed. RNA was extracted from cervical/thoracic and lumbar spinal cord tissue for qPCR and Nanostring gene expression analyses (n=5-9/group). LP 17 treatment studies were performed in an additional cohort of mice (n′=4/group). Human TREMi immunohistochemistry was performed on temporal lobe Human white matter brain biopsy tissue acquired from Dr. Vogel (Stanford Department of Pathology).

EAE Induction

EAE was induced in female C57BL/6 WT mice (9-13 weeks, Jackson Laboratories #00664) and Treml KO mice (12-22 weeks, bred in-house) as previously described using MOG3s . . . 55 emulsified in CF A (Hooke Laboratories, Lawrence, MA). Control WT mice received injections of CF A without MOG3s-ss (Hooke Laboratories, Lawrence, MA). Both EAE and control mice received two intraperitoneal pertussis toxin (PTX) injections (80-150 ng) 2 and 24 hours post MOG induction. Untouched, naive littermates were used as additional controls. EAE mice were weighed and scored from day 6 onwards and grouped by disease severity for each study (i.e., pre, low and high EAE). EAE severity score exceeding 4 was chosen as a final endpoint. All mice were housed under a 12 h light/dark schedule with ad libitum access to food and water and acclimatized for I week prior to experiments. All animal procedures were approved by so the Stanford Administrative Panel on Laboratory Animal Care (APLAC), accredited by the Association for the Assessment and Accreditation of Laboratory Animal Care (AAALAC International). All federal and state regulations governing the humane care and use of laboratory animals were upheld. For the subsequent experimental procedures, mice were anesthetized using isoflurane gas (2.0-3.0% for induction and I 0.5-2.5% for maintenance, unless otherwise noted).

Flow Cytometry

Following perfusion, brain, spinal cord, and splenic tissues were harvested before mechanical homogenization in ice cold CNS buffer (2.5% HEPES pH 7.5 [Invitrogen], in Hanks' Balance Salt Solution (HBSS) without Ca/Mg [Gibco]) and F ACS buffer (2% Fetal Bovine Serum in PBS), respectively. Samples were filtered through 40 μm cell strainers and centrifuged at 340 g (7 min. 4′C). For myelin removal, CNS samples were resuspended in standard isotonic percoll solution (Cytiva) and spun at 800 g (20 min. 4° C.) followed by a single wash with CNS buffer. Supernatant was aspirated and samples were resuspended in FACS buffer at a concentration of 1-2 million cells/sample. Cells were washed with PBS prior to staining with live/dead aqua (ThermoFisher Scientific, 20 min, RT). Samples were resuspended and incubated with the following fluorescently labeled antibodies (45 min, RT): APC TREMl (R&D Systems), PACBlue CDIIc (Biolegend). PE-Cy7 Ly-6G (Biolegend). APC-Cy7 CDIIb (Biolegend), PerCPCy5.5 CD45 (Bio legend). Samples were washed and PF A fixed (2% PFA, 20 min, RT). Cells were washed and resuspended in FACS buffer for final analysis. Tissues from EAE mice were harvested over 3 days to ensure an adequate n number of mice that fell within the pre, low or high EAE disease categories. All samples were run on the same day to reduce variance.

### APPENDIX 1

### DOTA Conjugation and [⁶⁴Cu]TREMl-mAb Radiosynthesis

Anti-mouse TREMl-mAb (R&D Systems) was conjugated with 1,4,7,10-tetraazacyclododecane-1,4,7,10-tetraacetic acid (DOTA) according to standard procedures using metal-free buffer as previously described (44). Briefly, DOTA-NHS ester (Macrocyclics Inc.) dissolved in dimethyl sulfoxide was incubated with mTREMl-mAb in HEPES buffer (0.1 mol/L, pH 8.8, 4° C.) and quenched after 14-16 h with biological grade TRJS buffer (pH 8.0, I M, Sigma). Excess DOTA-NHS was removed by Zeba Spin Desalting Columns (70K MVCO: ThermoFisher Scientific) and buffer-exchanged into ammonium acetate buffer (0.1 M. pH 5.5). DOTA-TREMl-mAb was concentrated (1.5-3.5 mg/mL) by ultrafiltration (Vivaspin, Sartorius), snap frozen, and stored at −80′C prior to radiolabeling. Liquid chromatography-mass spectrometry was used to determine an average of 2-3 DOT A molecules per antibody. Radiolabeling of DOTA-TREMl-mAb with ⁶⁴Cu (ty=12.7 h) was carried out using standard methods with some modifications (45). In brief, DOTA-TREMl-mAb dissolved in ammonium acetate buffer (0.1 M. pH 5.5) was incubated ⁶⁴CuCh solution (University of Wisconsin, Madison) with gentle shaking (300 rpm, pH 5.5, 30-60 min, 37° C.). EDT A (0.5 M, pH 8.0. Invitrogen) was added to scavenge unchelated ⁶⁴CuCh. Radiochemical purity was determined by instant thin-layer chromatography with TEC-Control Chromatography strips (Biodex Medical Systems). [⁶⁴Cu]TREMl-mAb was obtained with high molar activity (>2000 GBq/μmol). radiochemical purity (>99%). and labeling efficiency (98-99%), and formulated in phosphate buffered saline (0.1 M NaCl, 0.05 M sodium phosphate, pH 74).

[¹⁸FJGE-180 Radiosynthesis

[¹⁸F]GE-180 was synthesized as previously described (46). Chemical and radiochemical purity were determined by reverse-phase analytical HPLC (Phenomenex Luna column, 150×4.6 mm. particle size 3 μm. pore size 100 A) using a 11 min 50-95% gradient (0.1 mg/mL ascorbic acid in H2O:MeCN. monitored by gamma detection and UV at 254 and 280 nm). In vivo PET imaging studies Mice were anesthetized with isoflurane gas and intravenously injected with [⁶⁴Cu]TREM 1-mAb (90-120 μCi) or [¹⁸F]GE-180 (160-200 μCi) Ten min static PET images were acquired 18-21 and 50-60 min post-injection of [⁶⁴Cu]TREMl-mAb and [¹⁸F]GE-180 respectively using a dual microPET/CT scanner (Inveon, Siemens). A 3-dimensional ordered subsets expectation maximum (2 iterations) and MAP-OP (18 iterations) algorithm was utilized to reconstruct PET images (128×128×159 matrix size, 0.776×0.776×0 96 mm voxel size). CT images were acquired prior to each PET scan to provide an anatomical reference frame in addition to scatter and attenuation correction for PET data. Only EAE mice that fell within the pre. low or high EAE disease categories on experimental days were included for imaging studies. All mice successfully tail vein cannulated and injected with radiotracer were included in data analysis.

PET Image Analysis

PET images were analyzed using Vivoquant software (version 4.0. inviCRO) and visualized using Inveon Research Workspace (IRW, version 4.0: Siemens). Brain uptake was quantified as previously described (47). lo brief, a 3-dimensional mouse brain atlas was utilized to obtain tracer uptake values in a priori regions of interest (whole brain, pons. medulla. and cerebellum). Cervical/thoracic and lumbar spinal cord quantification was obtained via segmentation and exclusion of the vertebral column. Spleen, femur and heart RO Is were drawn manually using CT images as reference

Ex Vivo Gamma Counting and Autoradiography

Immediately following PET, tissues were collected for ex vivo biodistribution and autoradiography. Mice were deeply anesthetized (2.5-3.0% isoflurane) and blood samples were collected via cardiac puncture prior to PBS perfusion. Tissues of interest (heart, lung, liver, kidney, spleen, brain, spinal cord, and tail) were individually harvested, weighed, and gamma counted (Cobra II Auto-Gamma counter, Packard Biosciences Co, Hidex automatic gamma counter. Hidex). CNS tissues were further analyzed via digital autoradiography. Brain tissue was fresh frozen in optimum cutting temperature compound (O.C.T., Sakura Finetek Inc.) and sectioned at 20 μm using a cryostat (Microm). Slide-mounted brain sections and spinal cord tissues were exposed to a storage phosphor film (Fujifilm, GE Healthcare) for 10 half-lives at −20′C and scanned using a Typhoon phosphor imager (Amersham Biosciences). Brain section anatomy was confirmed by Nissl (cresyl violet acetate. Sigma Aldrich) staining as previously described (48) and analyzed with ImageJ (image processing software, version 2.0.0)

Blood Cytokine Analysis

Cardiac blood samples were placed in EDTA coated blood collection tubes (BD) and centrifuged (400-500 RCF) for 10 min Plasma was transferred to a sterile Eppendorf, spun at 13,000 RCF and stored at −80° C. Plasma samples were run by the Stanford Human Immune Monitoring Core facility using a 39-plex murine-specific Luminex array (Thermo Fisher Scientific).

RNA Extraction, cDNA Synthesis, and qRT-PCR

Spinal cords were flash frozen in Trizol (Invitrogen). Tissues were homogenized using a motorized handheld microtube homogenizer and centrifuged (10.000 RPM, 4° C.). RNA isolation followed the Trizol RNA extraction protocol. Nucleic acids isolation required chloroform (Sigma Aldrich). mRNA product was suspended in nuclease free water and assessed for concentration and quality using a BioSpectrometer (Eppendorf). This mRNA served as starting material for qPCR and Nanostring experiments. cDNA was synthesized using the RT2 First Strand kit (Qiagen). The synthesis reaction used 1250 ng of starting RNA material and incubation steps were completed in the Thermal Cycler Mini Amp (Applied Biosystem) following the kit protocol. All PCR reactions included 5 μL of SYBR green polymerase (Qiagen). 0.5 μL of specified RT-PCR primer. 1.5 μL of nuclease free water, and 3 μL of cDNA product. TSPO (Qiagen) and TREMl (Qiagen) primers were used. GAPDH was used as a housekeeping gene for all tissues. Reactions were completed in the applied biosystems QuantSh1dio 6 Real-Time PCR machine. Each sample was nm with three technical replicates. and fold change for each gene was calculated by deriving 2ddCT_Transcripts with undetectable values were assigned a cycle threshold of 38 for analysis as previously described (49). Samples with high variation between technical replicates (SD>0.70) were excluded from analysis.

### APPENDIX 1

Nanostring nCounter Technology

Modulation of neuroimmune gene signatures was assessed in gross lumbar spinal cord tissue (naive. n=5: WT EAE, n=10) using a customized nCounter neuroinflammation panel (Nanostring Technologies) which measures gene expression changes for over 700 neuroinflammation-related genes) Ten additional genes were added to the Neuroinflammation panel: (TSPO, GSK3b, PTGSI, CdI Ib, CD25, CD30. CD138, SIGMARI, TRPVI, RAGE). RNA samples (100 ng) were prepped following the of ficial hybridization protocol (https://www.nanostring.com/wp-content/uploads/2021/03/MAN-I 0056-05-Gene-ExpressionHybridization-Protocol.pdf) and reactions were incubated (18 h. 65-C) to ensure optimal hybridization. Hybridized RNA was diluted in 30 μL of nuclease free molecular grade water and loaded onto the SPTRING cartridge. Assay was completed on the SPRINT profiler as per manufacturer instruction. Through systematic review of the most differentially expressed genes, we identified 16 significantly up- or down-regulated genes, previously studied in MS/EAE pathology, and directly compared their expression change to that of TREM 1.

LPI 7 Treatment Studies

LPI 7 peptide (sequence: LQVTDSGL YRCVIYHPP) was synthesized by the Protein and Nucleic Acid Core Facility at Stanford University as previously described (J 9). WT EAE mice were induced with EAE and monitored daily as previously outlined. Once mice were considered pre-EAE (>I g weight loss in 48 h), mice were randomly split into LPI 7 10 mg/kg, LPI 7 15 mg/kg, and vehicle (saline) treatment groups. LP 17 or saline was administered daily (i.p) for 10 days and disease level was recorded.

### APPENDIX 1

Human TREMi Immunohistochemistry

Human control and MS brain Ff PE tissue sections were deparaffinized by heating (1 hr, 56° C.) and immediately passed through xylenes and a graded EtOH series from 100% I, 100% II, 95%, 70% into PBS (5 min per incubation). Antigen retrieval was performed by incubating tissue in a IO mM citrate buffer solution with 0.05% Tween 20 (pH 6.0) under low boil (20 min). Sections were cooled to RT then washed with PBS. Endogenous peroxidase activity was quenched by incubating in 2% H2O2 solution while shaking (20 min. RT). To further permeabilize tissue, sections were incubated in PBS with 0.3% Triton-X while shaking (2×10 min. RT). Tissue was blocked with 10% normal donkey serum (Jackson ImmunoResearch Laboratories. Inc.,) in PBS on a shaker (1 h, RT) and incubated (overnight, 4° C.) in rabbit polyclonal anti-TREM1 (Abeam) at 1:200 dilution. Tissue was then PBS washed (3×10 min) and incubated in secondary antibody solution (2 h, RT) while shaking. Biotinylated goat anti-rabbit (1:1000, Vector Laboratories) was used as secondary antibody. Sections were PBS washed (3×10 min) and incubated (1 h. RT) with Avidin-Biotin complex (VECTASTAIN Elite, Vector Laboratories). Sections were developed with the Vector 3,3′-diaminobenzidine (DAB) substrate kit (SK-4100). Sections were washed and nuclei were counterstained with hematoxylin (Abeam). Sections were dehydrated through a graded alcohol series, cleared in xylenes, and coverslipped with DPX mounting media (Sigma-Aldrich). Photomicrographs were acquired using a Keyence BZ-X710 microscope running BZ-X Viewer Software (Keyence).

Statistical Analysis

Statistical analyses of flow cytometry, in vivo PET, ex vivo biodistribution data, and qPCR was performed using GraphPad Prism (version 9.01). All data was assessed for normalization and parametric and non-parametric tests were applied as appropriate. Statistical analyses were performed using t-tests. one-way, and two-way analysis of variance (ANOVA) with multiple comparisons as indicated. Statistical analyses of cytokine and Nanostring data were performed in the Nanostring nSolver Advanced Analysis package and R version 4.0.2 with R studio version 1.1.453. Heatmaps were generated using Pheatmap (package version 1.3.1056). PCA was performed using the FactoMineR (package version 2.4 and visualized using the factoextra (package version 1.0.7). ROC curves were calculated and plotted using the plotROC (package version 2.2.1). Bivariate heatmap was generated using ggplot2 (package version 3.3.3) and correlation analysis with TREMl-PET was performed using GraphPad Prism (version 9.01).

### Example 8

Methods: Anti-TREM1 monoclonal antibody (mAb) was DOTA-conjugated and radiolabeled with copper-64 (64Cu). Static PET/CT images were acquired 3-40 hours after intravenous administration of 64Cu-TREM1-mAb to wild-type (WT) mice treated with 5 mg/kg LPS (LPS-Wr) or vehicle alone (Veh-WT). Gamma counting and autoradiography were conducted to confirm in vivo findings. RT-qPCR and flow cytometry were performed to assess alterations in TREM1 expression and cellular specificity in different tissues from LPS-WT versus Veh-WT mice. Luminex was used to investigate the relationship between TREM1-PET signal and inflammatory plasma cytokine signatures. Finally, the effect of genetically knocking out TREM1 on sickness behavior in LPS-injected mice was tested via survival studies and murine sepsis scoring.

Results: Quantification of TREM1-PET images revealed significantly higher signal in organs known to be affected by LPS challenge (brain, liver, lung, and spleen: p<0.01 vs Veh-WT), which was confirmed by ex vivo gamma counting and autoradiography. The specificity of 64Cu-TREM1-mAb was verified by its significantly lower binding in the brain, lungs, and spleen of LPS-treated-TREM1 knockout mice (LPS-K/O) versus LPS-WT mice (p<0.01), in addition to the relatively lower binding of 64Cu-Isotype-control in LPS-treated WT mice (LPS-1S0-WT). Flow cytometry demonstrated significant increases in TREM 1+ myeloid cells in the brain, lungs, and spleen of LPS-WT versus Veh-WT mice (p<0.01-p<0.0001), which was corroborated by RT-qPCR. Furthermore, TREM1-PET signal correlated with pro-inflammatory cytokine signatures and decreased survival of LPS injected mice.

Conclusion: 64Cu-TREM1-mAb is a highly promising myeloid cell-specific PET tracer with potential to shed light on the spatiotemporal dynamics of innate immune dysfunction in the whole body and brain across a broad range of diseases.

Materials and Methods

Study Design

Antibody-based PET tracers are highly effective for imaging immune responses, owing to their high target specificity and long biological half-lives (27). Here, we investigate the utility of 64Cu-TREM1-mAb for detecting toxic innate immune responses in a mouse model of LPS-induced sepsis. To identify the optimal time-point for imaging this model (i.e., the time post-tracer injection that affords the highest signal-to-background images), serial 10-minute static TREM1-PET imaging was performed at 3, 20, and 40 hours post-injection (hpi) of tracer, in a small cohort of wild-type (WT) mice that received an intraperitoneal (i.p.) injection of 5 mg/kg LPS (LPS-WT) or saline (vehicle [Veh]-WT). Tracer was injected 4-5 hours after mice received LPS or saline. Both systemic- and neuro-inflammation have been reported in this mouse model as early as 1 hour following i.p. LPS and maintained for at least 72 hours, with some reports that brain TNF-α levels remain elevated for months after a single i.p. injection (28,29). Following final PET/CT imaging with 64Cu-TREM1-mAb or a 64Cu-labeled isotype-control mAb (64Cu-Isotypecontrol-mAb, used to assess specificity of the TREM1-PET tracer), ex vivo gamma counting of tissues, and high-resolution autoradiography were conducted to confirm in vivo findings at the optimal imaging time-point. RT-qPCR was performed on mice from a separate cohort to assess levels of TREM1 in tissues known to be inflamed following LPS challenge (i.e., brain, liver, lungs, spleen) between 3-72 h. Flow cytometry was carried out using LPS-WT, Veh-WT, and TREM1-knockout (K/O) mice administered LPS (LPSK/0) mice to validate TREM1 as a specific marker of innate immune activation in the brain, lung, and spleen (30,31). The relationship between TREM1-PET signal and peripheral inflammatory plasma cytokine signatures was investigated using a bead-based immunoassay (i.e., Luminex). Finally, the effect of attenuating TREM1 signaling via genetic K/0 was studied by comparing the murine sepsis score and survival of WT compared to TREM1-K/O mice administered with LPS (see Supplemental Table 1 for number of mice per experiment).

Murine Model of LPS-Induced Sepsis

Female C57BU6 WT and TREM1-K/O mice (8-12 weeks, original breeders provided by Dr. Christoph Mueller, University of Bem) were housed under a 12-hour light/dark schedule with adlibitum food and water access. LPS (Escherichia coli lyophilized powder; Sigma) was dissolved in sterile saline immediately prior to i.p. injection (5 mg/kg). Veh-WT mice received equivalent volumes (by weight) of sterile saline. The Stanford Administrative Panel on Laboratory Animal Care (APLAC), which is accredited by the Association for the Assessment and Accreditation of Laboratory Animal Care (AAALAC International) and upholds all federal and state regulations governing the humane care and use of laboratory animals, approved all animal experiments.

64Cu-TREM1-mAb Radiosynthesis

Conjugation of anti-mouse anti-TREM1-mAb and isotype-control-mAb (R&D, IgG2A) with 1,4,7, 10-tetraazacyclododecane-1,4,7,10-tetraacetic acid (DOTA) and subsequent radiolabeling with 64Cu (half-life: 12.7 hours) was performed using standard procedures and metal-free buffers, as previously described (16,32,33). 64Cu-TREM1-mAb and 64Cu-Isotype-control were obtained with high molar activity (>0.400 MBq/μg), labeling efficiency (70-99%), and >99% radiochemical purity.

In Vivo PET/CT Imaging

LPS-WT, Veh-WT, and LPS-K/O mice were injected with 3.3-4.4 MBq of 64CuTREM1-mAb or 64Cu-Isotype-control-mAb (both formulated in phosphate-buffered saline) intravenously (i.v.) 4 hours following LPS injection. PET/CT images were acquired 19-20 hpi using a dual PET/CT scanner (Inveon; Siemens). Static PET images (10-min) were reconstructed using a 3-dimensional ordered subsets expectation maximization algorithm (16).

Image Analysis

PET and CT images were co-registered using Inveon Research Workplace image analysis software (v4.2; Siemens) and CT images used to manually determine liver, lung, and spleen regions of interest (ROIs). Brain PET quantification was performed using a semi-automated brain atlas approach in VivoQuant (version 3.0, inviCRO), as previously described (34). PET data is expressed as percent injected dose per gram (% ID/g).

Ex Vivo Gamma Counting and Autoradiography

Following PET, a blood sample was collected from each mouse via cardiac puncture immediately prior to transcardial perfusion. After perfusion, the heart, lungs, liver, spleen, kidney, and brain were dissected from each mouse, and gamma counting was performed using a Cobra II Auto-Gamma counter (Packard Biosciences Co.) to quantify % ID/g. Ex vivo high-resolution autoradiography was performed using 40 μm-thick brain and spleen sections (16). These sections were subsequently stained with cresyl violet (Sigma Aldrich) and hematoxylin and eosin (H&E, Fisher Scientific) to visualize regional tracer binding in brain and spleen respectively.

Flow Cytometry

Single cell suspensions were obtained from brain, lung, and spleen via mechanical homogenization following transcardial PBS perfusion. Live myeloid, lymphoid, and astrocyte populations were stained prior to 2% paraformaldehyde (ChemCruz) fixation and analyzed using FlowJo software (Tree Star Inc.).

Plasma Cytokine Analysis

Blood collected in EDTA-coated tubes (BO) was centrifuged (400-500 RCF, 10 minutes). Resulting plasma samples were analyzed by the Stanford Human Immune Monitoring Core using a 38-plex murine-specific Luminex array (eBiosciences/Affymetrix)

Survival and Behavioral Studies

A concentration of 15 mg/kg LPS was selected as the dose for all survival studies after obtaining pilot study results assessing morbidity rate following single injection of 5, 10, 15, or 20 mg/kg LPS. A single dose of 5 or 10 mg/kg did not lead to any morbidity within 24-48 h while 20 mg/kg had very potent effects of >50% morbidity within 24 h; 15 mg/kg was closest to, without exceeding, the dose that leads to death of 50% of mice (i.e., LD50). Female TREM1-K/O mice and WT littermates (20-23 weeks) were injected i.p. with LPS (15 mg/kg of LPS dissolved in saline). Mice were monitored daily for a week, and appearance (coat smoothness and piloerection) and activity level (natural or when provoked) assessed using a numerical murine sepsis severity scoring system (35).

Statistical Analysis

Graph Pad Prism (v9.01) was used to perform statistical analyses of flow cytometry, in vivo PET and ex vivo gamma counting data; R (v3.3.3) was used for cytokine analysis (Supplemental Methods). All data was assessed for normalization, and parametric and non-parametric tests were applied as appropriate. A p-value:50.05 was considered significant.

Results

TREM1-PET imaging enables specific non-invasive visualization of innate immune activation in the liver, lung, and spleen during LPS-induced sepsis. Pilot TREM 1-PET imaging of LPS-WT versus Veh-WT mice revealed no significant difference in signal in the liver or spleen at 3 hpi. Conversely, quantification of images at 20 and 40 hpi demonstrated significantly higher signal in both liver and spleen of LPSWT mice (liver p<0.0001, spleen: p<0.0001), without any substantial difference in signal-to-noise between time points. RT-qPCR data revealed higher levels of TREM1 in the liver, lungs, and spleen of LPS-WT compared to Veh-WT mice at 3-24h, with no significant difference at 72h. Hence, 20 hpi of 64CuTREM 1-mAb (−24h pi LPS) was chosen as the optimal timepoint to perform imaging for all subsequent studies.

Quantification of PET signal in peripheral tissues from a larger follow-up study revealed significantly elevated TREM1-PET signal in liver {p<0.0001), lungs (p=0.0005), and spleen (p=0.0003) of LPS-WT compared to Veh-WT mice. Importantly, LPSWT mice imaged with 64Cu-Isotype-control-mAb exhibited significantly reduced signal compared to LPS-WT mice imaged with 64Cu-TREM1-mAb (liver p<0.0001, lungs: p<0.0001, spleen: p=0.0011), confirming the specificity of 64Cu-TREM1-mAb. Specificity was further illustrated by significantly lower PET signal in the lungs of LPS-K/O mice (vs LPS-WT: p=0.0016), and the fact there was no significant difference in splenic signal between Veh-WT and LPS-K/O mice. In vivo findings were validated by ex vivo gamma counting of liver, lung, and splenic tissues and high-resolution autoradiography of the spleen overlaid with H&E staining. Autoradiography revealed a distinct pattern of 64Cu-TREM1-mAb binding restricted to the marginal zone and red pulp, which contain macrophages (36); uptake was not observed in the T- and B-cell-rich white pulp.

TREM 1-PET imaging was further corroborated by flow cytometry data, which showed significant increases in the frequency of the CD45hiCD11 b+ myeloid cells in the spleens of LPS-WT mice (versus Veh-WT: p=0.0033, and LPS-K/O mice: p=0.0018) with an upward trend observed in the lungs (Veh-WT:p=0.08, LPS-K/O: p=0.01). Notably, significant increases in TREM1+ cell frequency was observed in the lungs and spleen of LPS-WTs compared to Veh-WT (lungs: p=0.0016; spleen: p=0.0075) and LPS K/O (lungs; p=0.0001; spleen: p<0.0001) mice, with expression highly restricted to myeloid populations. Subsequent characterization of myeloid cells revealed constitutive TREM1 expression on splenic CD45hiCD11b+Ly6G+ neutrophils in Veh-WT and LPS-WT compared to LPS-K/O mice (Veh-WT: p<0.0001; LPS-WT: p<0.0001), with more significant expression detected in LPS-WT versus Veh-WT mice (p=0.0002). Similar results were observed in the lungs (vs LPS-K/O: LPSWT: p=0.0114; Veh-WT: p=0.059). Substantial TREM1 upregulation was also demonstrated on CD45hiCD11 b+Ly6G-monocyte/macrophages/dendritic cells (DCs) in LPS-WT mice (vs Veh-WT: lungs: p=0.0022, spleen: p<0.0001; vs LPS-K/O: lungs: p=0.0002, spleen: p<0.0001).

TREM1-PET imaging enables detection of subtle neuroinflammation during LPS Induced sepsis. Quantification of brain PET/CT images revealed significantly higher binding of 64Cu-TREM1-mAb in the whole brain of LPS-WT compared to both Veh-WT (p=0.0012) and LPS-ISO-WT (p=0.0005) mice. This is supported by significantly reduced signal in whole brain tissues of LPS-K/O vs LPS-WT animals, as assessed by gamma counting following perfusion to remove unbound intravascular tracer (p=0.0037). Regional quantification demonstrated significantly increased 64Cu-TREM1-mAb signal in the cortex (p=0.0313), hippocampus (p=0.0035), medulla (p=0.0017), midbrain (p=0.0169), and pons (p=0.0337) of LPS-WT versus Veh-WT mice. Signal also increased compared to LPS-ISO-WT in the hippocampus (p=0.0428), medulla (p=0.0011), and pons (p<0.0001). Reduced signal in LPS-K/O compared to LPS-WT mice was only observed in the medulla (p=0.0037) and not in the whole brain or other regions. High resolution ex vivo autoradiography of coronal brain sections demonstrated specific binding of 64Cu-TREM 1-mAb in the cerebellum, cortex, hippocampus, and medulla of LPS-WT mice. Moreover, flow cytometry and RT-qPCR further support these findings. Flow cytometry demonstrated a significant increase in CD45hiCD11 b+ myeloid cell infiltration into the brains of LPS-WT versus Veh-WT animals (p=0.0112). Conversely, resident co45midCD11 b+ microglia and CD45-CD11b-GFAP+ astrocyte populations did not demonstrate significant changes in frequency post-LPS treatment (p=0.8098 and p=0.0654 respectively). As in the periphery, increased TREM1+ cells were observed in the brains of LPS-WT animals (vs Veh-WT: p<0.0001; vs LPS-K/O: p<0.0001), with TREM1 predominantly expressed on myeloid cells. Brain TREM 1 upregulation in LPS-WT mice was observed on both CD4ShiCD11 b+Ly6G+ neutrophil (vs Veh-WT: p=0.0004; vs LPS-K/O: p<0.0001) and CD45hiCD11 b+Ly6G. monocyte/macrophage/DC (vs Veh-WT: p<0.0001; vs LPS-K/O: p<0.0001) populations. Resident brain co45midCD11b+ microglia showed a significant, yet comparatively low, increase in TREM1 (vs Veh-WT: p=0.0005; vs LPS-K/O: p<0.0005), as previously demonstrated after ischemia (16). Low levels of TREM 1 were also detected on co45hiCD11 b-lymphoid cells in WT mice compared to K/O mice (LPS-WT: p=0.0162; Veh-WT: p=0.0306). TREM1 was not significantly expressed on astrocytes. Increased TREM1 mRNA expression in the brains of LPS-WT compared to Veh-WT mice, at 3-24h after LPS challenge, reinforced these findings.

TREM1 is a biologically relevant imaging biomarker that reflects pro-inflammatory cytokine signatures and decreased survival. To further assess the relationship between TREM1-PET and peripheral inflammation, we performed unsupervised hierarchical clustering of plasma cytokine signatures. Three primary cytokine clusters were revealed in LPS-WT compared to Veh-WT mice: cluster 1 was upregulated, cluster 2 downregulated, and cluster 3 exhibited no changes. Notably, TREM1-PET signal in the spleen exhibited strong positive correlations with cluster 1 cytokines. Similar, but weaker, trends were observed for TREM1-PET signal in lungs, while TREM1-PET signal in the brain exhibited a significant correlation with only VEGF and MIP1 a cytokines in cluster 1. Automatic functional gene annotations indicated distinct biological roles for these clusters. The majority fraction of all cytokines in the three clusters were annotated with the terms “inflammatory response” and “immune response”. Over-representation analysis identified nine unique annotations as significantly over-represented from cluster 1, notably “response to LPS,” “monocyte chemotaxis,” and “chemokine-mediated signaling,” suggesting association with pro-inflammatory responses to LPS. “Cell response to TNF” was significantly enriched in cluster 1. Although “cell response to IL 1” was annotated in a similarly high fraction of cluster 1 genes, it did not reach significance. Given the link between TREM1 expression levels, TREM1-PET signal, and proinflammatory cytokine signatures in response to LPS-induced sepsis, we evaluated if excessive inflammation associated with TREM1 expression was linked to sickness behavior. Genetic knockout of TREM1 led to statistically-improved motor activity (p=0.0281-0.0031) and appearance (p=0.0424-0.0043) of LPS-K/O compared to LPSWT mice. Additionally, data from a small survival study (n=10) indicate improved survival in LPS-K/O mice by day 7 (55% vs 10%, p=0.1151). Investigation in larger cohorts is needed to further assess these promising results.

Discussion

Novel non-invasive methods permitting accurate detection of innate immune dysfunction are critical to enhance the understanding, diagnosis, and treatment of inflammatory disorders. Identifying specific biomarkers of innate immune cells and their functional phenotypes, paired with subsequent PET tracer development, is thus an area of great interest with important implications for a broad range of diseases. The most widely studied PET imaging agents for visualizing inflammation bind to TSPO—a protein localized on the outer mitochondrial membrane of myeloid and nonmyeloid cells (37,38). Although TSPO PET tracers have provided indispensable information regarding the link between inflammation and disease in vivo, both in rodent models and patients (23,39,40), the target itself has important limitations (41); these include its lack of cell specificity (i.e., it is expressed by endothelial cells, astrocytes, and myeloid cells to different extents depending on the context), as well as its high basal expression in peripheral organs, making it challenging to discern inflammation in systemic disorders. Importantly, it is unclear what a positive TS PO-PET signal represents in terms of beneficial and/or maladaptive immune responses (23,42). Other targets being explored for PET imaging of inflammation, including cyclooxygenase-2, cannabinoid receptor type 2, P2X purinoceptor 7, colony stimulating factor 1 receptor, and monoamine oxidase-B, also lack cell specificity, have high homeostatic basal expression, and/or fail to provide functionally relevant information regarding immune cell status (43). To overcome these limitations, we developed a PET tracer targeting TREM1, a highly specific biomarker of pro-inflammatory myeloid-driven immune responses (16). Here, we assessed the utility of this tracer in mice with LPS-induced sepsis, a well established model of systemic inflammation. PET imaging using 64Cu-TREM1-mAb enabled sensitive, non-invasive detection of activated myeloid lineage cells in the brain, lungs, and spleen of LPS-WT mice—organs known to be inflamed during sepsis (28,44-46). Tracer specificity was validated by low binding of 64Cu-TREM1-mAb and 64Cu Isotype-control-mAb in LPS-K/O and LPS-WT mice, respectively. Importantly, RT-qPCR data revealed there was indeed elevated TREM1 mRNA 3-24h after LPS challenge. Moreover, flow cytometry results indicated that TREM 1 protein expression was predominantly restricted to peripheral myeloid cells (i.e., DCs, macrophages, monocytes, and neutrophils) in LPS-induced septic mice.

Finally, we showed that TREM1 is a biologically relevant imaging biomarker reflecting pro-inflammatory immune responses and decreased survival, as the TREM1-PET signal correlated with pro-inflammatory cytokine signatures and TREM1 ablation prolonged survival following LPS administration. These results provide evidence for a strong biological connection between induction of myeloid TREM 1 expression and generation of toxic immune responses, further strengthening the rationale for pursuing TREM1 as a specific imaging biomarker of maladaptive inflammation (20,47-49). Importantly, TREM1 has previously been demonstrated to be critical in amplifying and regulating pro-inflammatory responses to microbial infection (50-53): upregulation on neutrophils and monocytes/macrophages has been reported in both patients experiencing microbial sepsis and mouse models of septic shock (50,52-54). Moreover, TREM1 modulators (including triptolide, LR12, and LP17) reduce pro-inflammatory mediators and increase survival in animal models of sepsis (55,56). Our finding of improved survival and reduced sickness behavior in LPS-K/O mice (compared to LPSWTs) further supports the functional relevance of TREM1 in the pathology of LPS-induced sepsis.

TREM 1 is emerging as a diagnostic and therapeutic target not only for sepsis (50,52-54), but also IBD (20), arthritis (21), and cancer (57). Most recently, we identified TREM1 as a major contributor to cerebral injury following stroke (16), indicating a role for this receptor in the pathophysiology of neurological disease. Whole-body TREM1-PET imaging revealed marked infiltration of peripheral myeloid cells into ischemic brain tissue, as well as increased myeloid cell activation in the spleen and, unexpectedly, intestines of stroked mice. TREM1-PET imaging can therefore be used as a powerful non-invasive tool to investigate the relationship between peripheral and central myeloid cell-driven immune responses in the context of different diseases.

Since innate immune dysfunction, and specifically myeloid cell activation, is at the heart of many devastating diseases, TREM 1-PET has enormous potential to be broadly applicable and reveal novel insights into the spatial and temporal dynamics of pathogenic myeloid cell responses in real time. Such discoveries will not only impact our basic understanding of disease but can also be utilized to improve the development and translation of immunomodulatory therapeutic approaches.

TREM1 is a highly specific imaging biomarker of pro-inflammatory myeloid cells and a potential therapeutic target in murine septic shock. We provide strong evidence that TREM 1-PET is a sensitive, accurate, and functionally relevant non-invasive imaging strategy for visualizing and quantifying whole body maladaptive myeloid cell activation in living subjects.

What is claimed is:

1. A method for detecting immune dysfunction in a subject, comprising administering to the subject of delivering an anti-TREM3 antigen binding molecule conjugated to one or more imaging or diagnostic substance(s), wherein detection of the imaging agent in an organ of the subject is an indication of immune dysfunction in the organ.

2. The method of claim 1, wherein the subject is in need of diagnosis.

3. The method of claim 1, wherein detection of the imaging agent in the brain of the subject identifies the location of an inflammation associated with a disease or condition comprising one or more of multiple sclerosis, Alzheimer's disease, Huntington's disease, Parkinson's disease, epilepsy, brain tumor, stroke, amyotrophic lateral sclerosis, spinal cord and/or brain trauma, a disease or condition which would benefit from enzyme replacement therapy (“ERT”), a neurological disease, chronic inflammatory conditions, acute inflammatory conditions, autoimmume disease, or infection.

4. The method of claim 1, wherein the subject is human.

5. The method of claim 1, wherein administration is through IV, intramuscular, subcutaneous, intraperitoneal, intravitreal, or intrathecal administration.

7. The method of claim 1, wherein the diagnostic substance comprises one or more PET and/or MRI probe(s), radiolabel(s) or isotope(s).

8. A composition comprising an anti-TREM3 antigen binding molecule conjugated to one or more imaging or diagnostic substance(s).

9. The composition of claim 8, wherein the diagnostic substance comprises one or more PET and/or MRI probe(s), radiolabel(s), or isotope(s).
